(19) United States
(12) Patent Application Publication
(10) Pub. No.: US 2026/0034202 A1
(43) Pub. Date: Feb. 5, 2026

(54) PAYLOAD DELIVERY SYSTEM

(71) Applicant: IMPERIAL COLLEGE INNOVATIONS LIMITED, London (GB)

(72) Inventors: Rongjun Chen, London (GB); Apanpreet K. Bhamra, London (GB); Robin Shattock, London (GB); Anna Blakney, London (GB)

(21) Appl. No.: 18/707,877

(22) PCT Filed: Nov. 10, 2022
(86) PCT No.: PCT/GB2022/052847

§ 371 Date: May 6, 2024

(30) Foreign Application Priority Data
Nov. 10, 2021 (GB) 2116125.2

(51) Int. Cl. A61K 39/145; A61K 39/00; A61P 37/04; C07K 14/005

(52) U.S. Cl. CPC A61K 39/145; A61P 37/04; C07K 14/005; A61K 2039/53; A61K 2039/6093; C12N 2760/16022; C12N 2760/16034; C12N 2760/16071

(57) ABSTRACT

The disclosure provides a sub-micron particle comprising a first payload molecule, a lipid structure and a plurality of amphiphilic polymer chains surrounding the lipid structure. The first payload molecule is a macromolecule, optionally a nucleic acid. Additionally, the hydrophobicity of the amphiphilic polymer chains changes in response to an external stimulus.

The present application is the U.S. National Stage of PCT/GB2022/052847, filed Nov. 10, 2022, which claims priority to Great Britain Patent Application No. 2116125.2, filed Nov. 10, 2021, the contents of each of which are incorporated herein by reference in their entireties.

The present invention relates to payload delivery systems, such as sub-micron particles, and in particular to sub-micron particles per se comprising a payload molecule, such as a macromolecule, for example nucleic acid. The invention extends to methods of producing the sub-micron particles, pharmaceutical compositions and vaccines comprising the sub-micron particles, and to medical uses thereof.

The project leading to this application has received funding from the European Union's Horizon 2020 research and innovation programme under the Marie Sklodowska-Curie grant agreement No 794059.

Research into vaccines and biotherapeutics based on nucleic acids, particularly messenger RNA (mRNA) and self-amplifying RNA (saRNA), has become increasingly popular over the last decade. mRNA-based vaccines and biotherapeutics represent a promising alternative to conventional approaches due to their high potency, capacity for rapid development, potential for low-cost manufacture, large-scale deployment, and safe administration. The mRNA vaccine and biotherapeutic field is developing extremely rapidly. The approval of the Pfizer/BioNTech and Moderna mRNA COVID vaccines marks the first time that in vitro transcribed mRNA has been approved for use in humans and makes mRNA a key asset in leading pharma and biotech pipelines. This new platform has tremendous transformational potential, not only as a vaccine platform against a range of infectious diseases but also for use as biotherapeutics against cancers, genetic disorders, and other diseases. saRNA is another popular choice thanks to its ability to produce 10 to 100-fold higher amounts of antigen from extremely small doses compared to mRNA¹ due to increased amounts of intracellular replication of the antigen-encoding saRNA. Many studies in animal models show foreign antigens expressed by saRNA viral vectors produce strong immune responses and provide prolonged protection in immunized animals against tumor cells and infectious agents².

However, clinical translation and commercialization of RNA systems are restricted by their inefficient in vivo delivery, high innate immunogenicity and instability. RNA systems, particularly mRNA and saRNA, have insufficient endocytosis due to their large molecular weights and high negative charge. Moreover, limited intracellular protein expression is an issue due to their endosomal entrapment and catalytic hydrolysis. Furthermore, RNA is very fragile and may readily degrade in exposure environments, thus requiring storage and distribution in a very challenging ultra-cold or cold chain.

New delivery systems are urgently needed to address these issues. Despite great technological advances, targeted intracellular delivery of nucleic acids including RNA remains a challenge. Viral vectors are known to have high transfection efficiencies; however, the clinical use of such delivery systems is limited due to challenges in safety concerns, large-scale production and the need for more rapid development. Therefore, non-viral vectors, such as cationic lipid nanoparticles (LNPs), are a promising alternative. Nevertheless, conventional nanoparticles have poor stability, high immunogenicity and unfavorable biodistribution, limiting clinical applications.

The present invention arises from the inventors' work in attempting to overcome the problems associated with the prior art.

In accordance with a first aspect of the invention, there is provided a sub-micron particle comprising a first payload molecule, a lipid structure and a plurality of amphiphilic polymer chains surrounding the lipid structure, wherein the first payload molecule is a macromolecule, optionally a nucleic acid, and the hydrophilicity of the amphiphilic polymer chains changes in response to an external stimulus.

The inventors have found that sub-micron particle of the first aspect can address issues associated with the prior art by using functional polymers and targeting ligands to provide more controlled and targeted delivery. As discussed in the examples, the sub-micron particles developed by the inventors have shown effective cytoplasmic delivery of functional macromolecular payloads, such as saRNA as well as co-delivery of saRNA and small-molecule payload, such as Ruxolitinib. The inventors have also found that the sub-micron particles can stabilise the macromolecular payload, such as a nucleic acid, allowing long-term stable storage of formulations both lyophilised and in aqueous solution at room and tropical temperatures. Accordingly, this eliminates the need for ultra-cold or cold storage.

The term “sub-micron” can be understood to mean that the particle of the invention has a largest maximum dimension of less than 1 μm. More preferably, the maximum dimension of the particle is less than 900 nm, less than 800 nm, less than 700 nm or less than 600 nm, and most preferably less than 500 nm, less than 400 nm, less than 300 nm or less than 200 nm. The sub-micron particle may have a largest maximum dimension of between 10 and 900 nm, between 20 and 800 nm, between 30 and 700 nm or between 40 and 600 nm, more preferably between 50 and 500 nm or between 60 and 400 nm, and most preferably between 80 and 300 nm or between 100 and 200 nm. The largest maximum dimension of the sub-micron particle may correspond to the Z-average size as determined using Zetasizer μV instrument.

The first payload molecule is preferably encapsulated in the lipid structure. Alternatively or additionally, the first payload molecule may be covalently conjugated and/or physically attached to the outer surface of the lipid structure. Physical attachment, as described anywhere herein, may be by electrostatic interaction and/or hydrophobic interaction.

Suitable macromolecules, which may be used as the first payload molecule, preferably have a molecular weight which is at least 900 daltons, at least 1000 daltons or at least 1200 daltons.

The macromolecule may be a nucleic acid, a peptide, an affimer, a protein, an antibody or a fragment thereof, a glycoprotein, a lipopolysaccharide or a carbohydrate. The macromolecule may be a lipid or a macrocycle.

Preferably, however, the first payload molecule is a nucleic acid. The nucleic acid may be DNA, RNA or a DNA/RNA hybrid sequence. Preferably, the nucleic acid is DNA or RNA. Most preferably, the nucleic acid is RNA. The RNA may be single stranded or double stranded. The RNA may be selected from the group consisting of: messenger RNA (mRNA); self-amplifying RNA (saRNA); antisense RNA (asRNA); RNA aptamers; interference RNA (RNAi); micro RNA (miRNA); short interfering RNA (siRNA); short hairpin RNA (shRNA); and small RNA.

Preferably, the RNA is self-amplifying RNA (saRNA). Preferably, the RNA is messenger RNA (mRNA). The skilled person would appreciate that self-amplifying RNAs may contain the basic elements of mRNA (a cap, 5′ UTR, 3′UTR, and poly(A) tail of variable length), but may be considerably longer (for example 9-12 kb).

The nucleic acid sequence, preferably RNA, may be at least 10 bases in length, at least 20 bases in length, at least 50 bases in length, at least 100 bases in length, at least 200 bases in length, at least 300 bases in length, at least 400 bases in length, at least 500 bases in length, at least 600 bases in length at least 700 bases in length, at least 800 bases in length or at least 900 bases in length.

In one preferred embodiment, the RNA is saRNA or mRNA.

The nucleic acid sequence, preferably RNA, and most preferably saRNA or mRNA, may be at least 1000 bases in length, at least 2000 bases in length, at least 3000 bases in length, at least 4000 bases in length, at least 5000 bases in length, at least 6000 bases in length, at least 7000 bases in length, at least 8000 bases in length, at least 9000 bases in length at least 10000 bases in length, at least 11000 bases in length or at least 12000 bases in length.

In one embodiment, the nucleic acid sequence is at least 6000 bases in length. In one embodiment, the RNA is at least 6000 bases in length. In a preferred embodiment, the saRNA is at least 6000 bases in length.

In an alternative embodiment, the nucleic acid sequence is at least 900 bases in length. In one embodiment, the RNA is at least 900 bases in length. In a preferred embodiment, the mRNA is at least 900 bases in length.

The nucleic acid sequence, preferably RNA, and most preferably saRNA, may be between 5000 and 20000 bases in length, between 5500 and 15000 bases in length, between 6000 and 14000 bases in length, between 6500 and 13000 bases in length, between 7000 and 12000 bases in length, between 8000 and 11000 bases in length, between 9000 and 10000 bases in length.

Alternatively, the nucleic acid sequence, preferably RNA, and most preferably mRNA, may be between 50 and 10000 bases in length, between 100 and 9000 bases in length, between 200 and 8000 bases in length, between 300 and 7000 bases in length, between 400 and 6000 bases in length, between 500 and 6000 bases in length, between 600 and 5000 bases in length, between 700 and 4000 bases in length, between 800 and 3000 bases in length or between 900 and 2000 bases in length.

In one embodiment, the nucleic acid sequence is between 6000 and 15000 bases in length. The nucleic acid sequence may be between 8000 and 12000 bases in length. The RNA may be between 6000 and 15000 bases in length. The RNA may be between 8000 and 12000 bases in length. Preferably, the saRNA is between 6000 and 15000 bases in length. Preferably the saRNA is between 8000 and 12000 bases in length.

In an alternative embodiment, the nucleic acid sequence is between 400 and 14000, between 500 and 10000, between 600 and 7500, between 700 and 5000, between 800 and 4000 or between 900 and 2000 bases in length. The RNA may between 400 and 14000, between 500 and 10000, between 600 and 7500, between 700 and 5000, between 800 and 4000 or between 900 and 2000 bases in length. Preferably, the mRNA is between 400 and 14000, between 500 and 10000, between 600 and 7500, between 700 and 5000, between 800 and 4000 or between 900 and 2000 bases in length.

The skilled person would appreciate that when the nucleic acid is double stranded, for example double stranded RNA or DNA, “bases in length” will refer to the length of base pairs.

The lipid structure may be a lipid nanoparticle or a liposome. Preferably, the or each lipid structure is a lipid nanoparticle.

In some embodiments, the lipid structure may comprise a plurality of lipids.

At least one of the plurality of lipids may comprise a cationic or ionizable lipid. The cationic or ionizable lipid may be a multivalent cationic lipid. The cationic or ionizable lipid may be a pH-sensitive lipid. The cationic or ionizable lipid may comprise a positively charged or ionizable nitrogen atom. The cationic or ionizable lipid may display a positive charge in an acidic solution. A solution may be understood to be acidic if it has a pH of less than 7 at 20° C., more preferably less than 6.5 at 20° C. A solution may be understood to be acidic if it has a pH of between 3.5 and 7 at 20° C. or between 4 and 7 at 20° C., more preferably between than 4.5 and 6.5 at 20° C.

The cationic or ionizable lipid may be dioleoyl-3-trimethylammonium propane (DOTAP), 1,2-di-O-octadecenyl-3-trimethylammonium propane (DOTMA), an ethylphosphatidylcholine (ethyl PC), didodecyldimethylammonium bromide (DDAB), 3β-[N—(N′,N′-dimethylaminoethane)-carbamoyl]cholesterol (DC-Cholesterol), N4-Cholesteryl-Spermine (GL67), 1,2-dioleyloxy-3-dimethylaminopropane (DODMA), DLin-MC3-DMA, 1,2-dioleoyl-3-dimethylammonium-propane (DODAP), or heptadecan-9-yl 8-((2-hydroxyethyl) (6-oxo-6-(undecyloxy) hexyl) amino) octanoate (SM-102). In some embodiments, the cationic or ionizable lipid is dioleoyl-3-trimethylammonium propane (DOTAP). In some embodiments, the cationic or ionizable lipid is DLin-MC3-DMA. In some embodiments, the cationic or ionizable lipid is DODAP.

The cationic or ionizable lipid may comprise at least 5 wt %, at least 10 wt %, at least 20 wt %, at least 30 wt % at least 40 wt %, at least 50 wt % or at least 55 wt % of the plurality of lipids. The cationic or ionizable lipid may comprise between 5 and 80 wt % or between 10 and 60 wt % of the plurality of lipids. In some embodiments, the cationic or ionizable lipid may comprise between 20 and 50 wt % or between 25 and 35 wt % of the plurality of lipids. In alternative embodiments, the cationic or ionizable lipid may comprise between 30 and 70 wt %, between 40 and 65 wt % or between 50 and 60 wt % of the plurality of lipids.

The lipid structure and/or the plurality of lipids may comprise one or more helper lipids. In one embodiment, the helper lipids may be zwitterionic, such as phosphatidylcholine (including DOPC, DSPC, DPPC, DDPC, DLPC, DMPC, POPC, DEPC, L-α-phosphatidylcholine) or phosphatidylethanolamine (including DOPE, DMPE, DPPE, DSPE).

Alternatively, in another embodiment, the helper lipids may be non-zwitterionic, such as phosphatidylglycerol (including DOPG, DMPG, DPPG, DSPG, POPG), phosphatidylserine (including DOPS) and phosphatidic acid (including DMPA, DPPA, DSPA). Hence, in some embodiments, at least one of the plurality of lipids may not comprise a zwitterionic lipid. In other words, therefore, the sub-micron particles of the invention may not contain or comprise zwitterionic lipids. However, in other embodiments, at least one of the plurality of lipids may comprise a zwitterionic lipid. The zwitterionic lipid may contain an equal number of positively- and negatively-charged functional groups and display a net charge of zero at a zwitterionic pH. The zwitterionic pH may vary depending upon the zwitterionic lipid. In some embodiments, a zwitterionic pH may be understood to be and acidic pH. Accordingly, a zwitterionic pH may be understood to be a pH of between 1 and 8 at 20° C., between 2 and 7 at 20° C., between 3 and 6 at 20° C., between 4 and 5 at 20° C. or between 4.3 and 4.5 at 20° C.

The zwitterionic lipid may comprise a positively charged nitrogen atom. The zwitterionic lipid may comprise a negatively charged oxygen atom. The oxygen atom may be bonded to a P(O) group or to a C(O) group. The zwitterionic lipid may be phosphatidylcholine (including DOPC, DSPC, DPPC, DDPC, DLPC, DMPC, POPC, DEPC, L-α-phosphatidylcholine) or phosphatidylethanolamine (including DOPE, DMPE, DPPE, DSPE). The zwitterionic lipid may be 1,2-dioleoyl-sn-glycero-3-phosphoethanolamine (DOPE) or L-α-phosphatidylcholine (PC). In some embodiments, the zwitterionic lipid is 1,2-dioleoyl-sn-glycero-3-phosphoethanolamine (DOPE). The non-zwitterionic lipid may be phosphatidylglycerol (including DOPG, DMPG, DPPG, DSPG, POPG), phosphatidylserine (including DOPS) and phosphatidic acid (including DMPA, DPPA, DSPA).

The weight ratio of the cationic or ionizable lipid to the helper lipid may be between 20:1 and 1:20, between 10:1 and 1:10 or between 5:1 and 1:5. In some embodiments, the weight ratio of the cationic or ionizable lipid to the helper lipid may be between 4:1 and 1:4, between 3:1 and 1:3, between 2:1 and 1:2, between 1.5:1 and 1:1.5 or between 1.8:1 and 1:1.8. In alternative embodiments, the weight ratio of the cationic or ionizable lipid to the helper lipid may be between 1:1 and 1:10, between 1:2 and 1:8 or between 1:4 and 1:5. In some embodiments, the weight ratio of the cationic or ionizable lipid to the helper lipid is about 1:1.

The helper lipid may comprise at least 5 wt %, at least 10 wt %, at least 20 wt % or at least 30 wt % of the plurality of lipids. In some embodiments, the helper lipid may comprise at least 40 wt % or at least 50 wt % of the plurality of lipids. The helper lipid may comprise between 5 and 80 wt % or between 10 and 70 wt % of the plurality of lipids. In some embodiments, the helper lipid may comprise between 20 and 50 wt % or between and 35 wt % of the plurality of lipids. In alternative embodiments, the helper lipid may comprise between 30 and 60 wt % or between 50 and 60 wt % of the plurality of lipids. In further alternative embodiments, the helper lipid may comprise between 2 and 40 wt %, between 4 and 30 wt %, between 6 and 25 wt %, between 8 and 20 wt % or between 10 and 15 wt % of the plurality of lipids.

At least one of the plurality of lipids may comprise sterol. The sterol may be or comprise a C₁₋₂₄ alkyl phytosterol, stigmasterol or stigmastanol. Preferably, the sterol is cholesterol.

The sterol may comprise at least 1 wt %, at least 2 wt %, at least 5 wt %, at least 10 wt %, at least 20 wt %, at least 30 wt % or at least 40 wt % of the plurality of lipids. The sterol may comprise 1 and 80 wt %, between 2 and 70 wt % or between 5 and 60 wt % of the plurality of lipids. In some embodiments, the sterol may comprise between 10 and 55 wt %, between 20 and 50 wt %, between 30 and 45 wt % or between 35 and 45 wt % of the plurality of lipids. In alternative embodiments, the sterol may comprise between 2 and 40 wt %, between 4 and 30 wt %, between 5 and 20 wt %, between 6 and 15 wt % or between 7 and 10 wt % of the plurality of lipids. In alternative embodiments, the sterol may comprise between 10 and 50 wt %, between 20 and 40 wt % or between 25 and 30 wt % of the plurality of lipids.

At least one of the plurality of lipids may comprise a PEGylated lipid. The PEGylated lipid may be understood to be a lipid which has been modified to comprise a polyethylene glycol (PEG) group or a derivative thereof. The derivative of PEG may be understood to be a multi-arm PEG, a functionalised PEG, etc. In some embodiments the shield lipid may be a DMG, DSPE or DPPC lipid which has been modified to comprise a PEG group or its derivatives. Accordingly, the PEGylated lipid may be DMG-PEG, PEG-DSPE or PEG-DPPC. The PEG group may have an average molecular weight of between 100 and 20,000 Da, between 200 and 15,000 Da, between 300 and 10,000 Da, between 400 and 7,500 Da or between 500 and 5,000 Da. In some embodiments, the PEG group may have an average molecular weight of between 1,000 and 3,000 Da, between 1,250 and 1,750 Da, between 1,500 and 2,500 Da, between 1,750 and 2,250 Da or between 1,900 and 2,100 Da. In some embodiments, the PEG group may have an average molecular weight of between 2,000 and 8,000 Da, between 3,000 and 7,000 Da, between 4,000 and 6,000 Da, between 4,500 and 5,500 Da or between 4,750 and 5,250 Da. The average molecular weight may be a number average molecular weight or a weight average molecular weight.

The PEGylated lipid may comprise at least 0.1 wt %, at least 0.5 wt %, at least 1 wt %, at least 2 wt %, at least 3 wt %, at least 4 wt % or at least 5 wt % of the plurality of lipids. The PEGylated lipid may comprise 0.1 and 50 wt %, between 0.5 and 40 wt %, between 1 and 30 wt %, between 2 and 20 wt %, between 3 and 10 wt %, between 4 and 8 wt % or between 5 and 7 wt % of the plurality of lipids.

The sub-micron particle may have an N/P ratio of at least 1:100, at least 1:50, at least 1:10 or at least 1:5, more preferably at least 1:3, at least 1:2 or at least 1:1, and most preferably at least 2:1, at least 3:1, at least 4:1, at least 5:1, at least 6:1, at least 7:1, at least 8:1 or at least 9:1. The sub-micron particle may have an N/P ratio of less than 100:1, less than 50:1, less than 40:1, less than 30:1, less than 28:1, less than 26:1, less than 24:1, less than 22:1, less than 21:1 or less than 20:1. The sub-micron particle may have an N/P ratio of between 1:100 and 100:1, between 1:50 and 80:1, between 1:10 and 60:1, between 1:5 and 50:1, between 1:3 and 40:1, between 1:2 and 30:1, between 1:1 and 25:1, between 2:1 and 20:1, between 5:1 and 15:1, or between 8:1 and 12:1. In preferred embodiment, the sub-micron particle has an N/P ratio of between 9:1 and 11:1. The N/P ratio may be understood to be the ratio between cationic amines in the lipid structure and anionic phosphates in the payload molecule.

The ratio of first payload molecule (preferably nucleic acid, more preferably RNA) to lipids may be between 2.5×10⁻⁹:1 to 0.25:1.

The external stimulus may be a change in pH, a change in temperature and/or exposure to light. Accordingly, the hydrophilicity of the amphiphilic polymer chains may decrease in response to a reduction in pH. Alternatively, the hydrophilicity of the amphiphilic polymer chains may decrease in response to an increase in temperature or exposure to light.

Preferably, the polymer chains permeabilize the lipid membrane in response to the external stimulus. The external stimulus may be a chemical stimulus, which may be selected from a change in pH, a specific redox potential, a specific ion or a specific gas.

Alternatively, the stimulus may be a physical stimulus, which may be selected from a temperature variation, a change in light or an electromagnetic field. The stimulus may alternatively be a biochemical stimulus, which may be selected from a protein, a peptide, an enzyme, a glucose or a nucleic acid, such as DNA.

In some embodiments, the external stimulus is a change in pH.

Advantageously, pH responsive polymers in particular can mimic the anionic peptides on the surface of viruses that play a role in membrane destabilization, allowing endosomal escape of payloads into the cell cytoplasm. As discussed in the examples, the inventors have shown that pH-responsive polymers can have endosomolytic activity and can facilitate the cytoplasmic delivery of payloads when used as part of a sub-micron particle of the invention.

The plurality of amphiphilic polymer chains may be or comprise polymers as described in WO 2018/011580 A1, WO 2021/260392 A1 and/or Chen et al.⁴. The amphiphilic polymers may be responsive to an external stimulus. The external stimulus may be pH.

WO 2021/260392 A1 relates a new polymerisation method and the use of this method to synthesize novel polymers including polyesters, polyethers, and polyester-ethers. It has been reported those types of polymers could be temperature responsive. Other temperature-responsive polymers may include or comprise poly(N-isopropylacrylamide-co-methacrylic acid).

Additionally, the polymers could be conjugated with some other component(s), where appropriate or needed, through a linker in response to a stimulus such as light, heat, enzyme or others. One example is a membrane-permeabilizing polymer could be made membrane-inactive by conjugation through a “shielding” component. Upon arrival at a target site, the linker could be cleaved upon a specific stimulus and the polymer will be transformed into a membrane-permeabilizing state. Alternatively, those polymers could also be conjugated with one or more components that are responsive to a stimulus such as pH, light, heat, enzyme or others.

The amphiphilic polymer chains may be negatively charged at a pH of at least 4 at 20° C., at least 5 at 20° C., at least 6 at 20° C., at least 6.5 at 20° C. or at least 7 at 20° C. The amphiphilic polymer chains may be negatively charged at a pH of between 4 and 10 at 20° C., between 5 and 9 at 20° C., between 6 and 8 at 20° C. or between 6.5 and 7.5 at 20° C.

The amphiphilic polymer chains may comprise at least one COOH group. It may be appreciated that the COOH group may be deprotonated at a pH above its pKa, i.e. is COO⁻, and the amphiphilic polymer is therefore relatively hydrophilic. However, at a pH below its pKa, the COO⁻ group may be protonated, i.e. is COOH, and the amphiphilic polymer is therefore more hydrophobic. Accordingly, the hydrophilicity of the group decreases at lower pHs.

The amphiphilic polymer chains may comprise one or more mers of formula I:

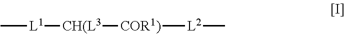

wherein L¹ comprises one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴;
L² and L³ are independently absent or comprise one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴;
each R¹ is independently NR⁶R⁷, OR¹⁰ or OH;
R⁴ is H, an optionally substituted C₁₋₃₀ alkyl, an optionally substituted C₂₋₃₀ alkenyl or an optionally substituted C₂₋₃₀ alkynyl;
R⁶ and R⁷ are each independently H, an optionally substituted C₁₋₃₀ alkyl, an optionally substituted C₂₋₃₀ alkenyl or an optionally substituted C₂₋₃₀ alkynyl; and
R¹⁰ is an optionally substituted C₁₋₃₀ alkyl, an optionally substituted C₂₋₃₀ alkenyl or an optionally substituted C₂₋₃₀ alkynyl.

In some embodiments, the amphiphilic polymer chains may comprise two or more mers of formula I which can be the same or different to each other.

Preferably, in at least one of the mers of formula I, R¹ is OH.

In some embodiments, the amphiphilic polymer chains may comprise one or more mers of formula II:

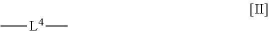

wherein L⁴ comprises one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴; and
R⁴ is as defined above.

In some embodiments, the amphiphilic polymer chains may comprise two or more mers of formula II which can be the same or different to each other.

The amphiphilic polymer chains may have a random sequence. Alternatively, the amphiphilic polymer chains may have a controlled sequence. For instance, the amphiphilic polymer chains may be block copolymers.

In some embodiments, the amphiphilic polymer chains are or comprise a plurality of polymers of formula III:

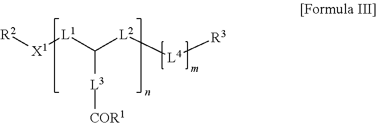

wherein L to L⁴, R¹, R⁴, R⁶ and R⁷ are as defined above;
X¹ is absent or is O, S or NR⁵;
R² is H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle;
R³ and R⁵ are each independently H, an optionally substituted C₁₋₃₀ alkyl, an optionally substituted C₂₋₃₀ alkenyl or an optionally substituted C₂₋₃₀ alkynyl; and
n is an integer and is at least 1; and
m is 0 or is an integer which is at least 1.

There may be one or more -L⁴- blocks (which may be same or different from each other).

There may be one or more -L¹-CH(L³-COR¹)-L²- blocks (which may be same or different from each other).

The polymers may have a random or controlled sequence.

The term “alkyl” as used herein, unless otherwise specified, refers to a saturated straight or branched hydrocarbon. The alkyl may be a primary, secondary, or tertiary hydrocarbon. C₁₋₃₀ alkyls include for example methyl, ethyl, n-propyl (1-propyl), isopropyl (2-propyl, 1-methylethyl), butyl, pentyl, hexyl, isobutyl, sec-butyl, tert-butyl, isopentyl, neopentyl, isohexyl, heptyl, octyl, nonyl, decyl, undecyl and dodecyl. The or each alkyl may be an optionally substituted C₁₋₃₀ alkyl, an optionally substituted C₁₋₂₀ alkyl, an optionally substituted C₁₋₁₂ alkyl, an optionally substituted C₁₋₆ alkyl or an optionally substituted C₁₋₃ alkyl. An alkyl group can be unsubstituted or substituted. A substituted alkyl may be substituted with one or more substituents selected from the group consisting of halogen, CN, NO₂, COOR⁸, NR⁸R⁹, OR⁸, SR⁸, SSR⁸, CONR⁸R⁹, N₃, OP(O)OR⁸R⁹, PR⁸R⁹, oxo, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle, wherein R⁸ and R⁹ are each independently H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle.

The term “alkylene”, as used herein, unless otherwise specified, refers to a bivalent saturated straight or branched hydrocarbon. An optionally substituted alkylene is analogous to an optionally substituted alkyl, as defined above, but with a hydrogen atom removed therefrom to make the alkyl group bivalent.

The term “alkenyl” refers to olefinically unsaturated hydrocarbon groups which can be unbranched or branched. Accordingly, an alkenyl may contain one or more double bonds between adjacent carbon atoms. C₂-C₃₀ alkenyl includes for example vinyl, allyl, propenyl, butenyl, pentenyl and hexenyl. The or each alkenyl may be an optionally substituted C₂₋₂₀ alkenyl, an optionally substituted C₂₋₁₂ alkenyl, an optionally substituted C₂₋₆ alkenyl or an optionally substituted C₂₋₃ alkenyl. An alkenyl group can be unsubstituted or substituted with one or more substituents selected from the group consisting of halogen, CN, NO₂, COOR⁸, NR⁸R⁹, OR⁸, SR⁸, SSR⁸, CONR⁸R⁹, N₃, OP(O)OR⁸R⁹, PR⁸R⁹, oxo, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle, wherein R⁸ and R⁹ are each independently H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle.

The term “alkenylene”, as used herein, unless otherwise specified, refers to a bivalent olefinically unsaturated straight or branched hydrocarbon. An optionally substituted alkenylene is analogous to an optionally substituted alkenyl, as defined above, but with a hydrogen atom removed therefrom to make the alkyl group bivalent.

The term “alkynyl” refers to acetylenically unsaturated hydrocarbon groups which can be unbranched or branched. Accordingly, an alkynyl may contain one or more triple bonds between adjacent carbon atoms. An alkynyl group may further contain one or more double bonds between adjacent carbon atoms. The C₂-C₃₀ alkynyl includes for example propargyl, propynyl, butynyl, pentynyl and hexynyl. The or each alkynyl may be an optionally substituted C₂₋₃₀ alkynyl, an optionally substituted C₂₋₂₀ alkynyl, an optionally substituted C₂₋₁₂ alkynyl, an optionally substituted C₂₋₆ alkynyl or an optionally substituted C₂₋₃ alkynyl. An alkynyl group can be unsubstituted or substituted with one or more substituents selected from the group consisting of halogen, CN, NO₂, COOR⁸, NR⁸R⁹, OR⁸, SR⁸, SSR⁸, CONR⁸R⁹, N₃, OP(O)OR⁸R⁹, PR⁸R⁹, oxo, an optionally substituted C₂₋₃₀ alkenyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle, wherein R⁸ and R⁹ are each independently H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle.

The term “alkynylene”, as used herein, unless otherwise specified, refers to a bivalent acetylenically unsaturated straight or branched hydrocarbon. An optionally substituted alkynylene is analogous to an optionally substituted alkynyl, as defined above, but with a hydrogen atom removed therefrom to make the alkyl group bivalent.

“Aryl” refers to an aromatic 6 to 20 membered hydrocarbon group. Examples of a C₆-C₂₀ aryl group include, but are not limited to, phenyl, α-naphthyl, β-naphthyl, biphenyl, tetrahydronaphthyl and indanyl. The or each aryl may be an optionally substituted C₆₋₁₂ aryl.

“Cycloalkyl” refers to a non-aromatic, saturated, monocyclic, bicyclic or polycyclic hydrocarbon 3 to 20 membered ring system. The or each cycloalkyl may be an optionally substituted C₃₋₂₀ cycloalkyl, an optionally substituted C₃₋₁₂ cycloalkyl or an optionally substituted C₃₋₆ cycloalkyl. Representative examples of a cycloalkyl include, but are not limited to, cyclopropyl, cyclobutyl, cyclopentyl, cyclohexyl.

“Cycloalkenyl” refers to a non-aromatic, olefinically unsaturated, monocyclic, bicyclic or polycyclic hydrocarbon 3 to 20 membered ring system. Accordingly, a cycloalkenyl may contain one or more double bonds between adjacent carbon atoms in the ring. The or each cycloalkenyl may be an optionally substituted C₃₋₂₀ cycloalkenyl, an optionally substituted C₃₋₁₂ cycloalkenyl or an optionally substituted C₃₋₆ cycloalkenyl.

“Cycloalkynyl” refers to a non-aromatic, acetylenically unsaturated, monocyclic, bicyclic or polycyclic hydrocarbon 3 to 20 membered ring system. Accordingly, a cycloalkynyl may contain one or more triple bonds between adjacent carbon atoms in a ring. A cycloalkenyl may also contain one or more double bonds between adjacent carbon atoms in a ring. The or each cycloalkynyl may be an optionally substituted C₃₋₂₀ cycloalkynyl, an optionally substituted C₃₋₁₂ cycloalkynyl or an optionally substituted C₃ 6 cycloalkynyl.

“Heteroaryl” refers to a monocyclic or bicyclic aromatic 5 to 20 membered ring system in which at least one ring atom is a heteroatom. The or each heteroatom may be independently selected from the group consisting of oxygen, sulfur, nitrogen and phosphorous. The heteroaryl may be an optionally substituted 5 to 20 membered heteroaryl or an optionally substituted 5 to 10 membered heteroaryl. Examples of 5 to membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic heteroaryl groups include for example those where a phenyl, pyridine, pyrimidine, pyrazine or pyridazine ring is fused to a 5 or 6-membered monocyclic heteroaryl ring.

“Heterocycle” or “heterocyclyl” refers to 3 to 20 membered monocyclic, bicyclic, polycyclic or bridged molecules in which at least one ring atom is a heteroatom. The or each heteroatom may be independently selected from the group consisting of oxygen, sulfur, nitrogen and phosphorous. A heterocycle may be saturated or partially saturated. A heterocyclic group may be an optionally substituted 3 to 20 membered heterocycle or an optionally substituted 3 to 12 membered heterocycle. Exemplary 3 to membered heterocycle groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane, and oxazine.

An aryl, cycloalkyl, cycloalkenyl, cycloalkynyl, heteroaryl or heterocycyl group can be unsubstituted or substituted with one or more substituents selected from the group consisting of halogen, CN, NO₂, COOR⁸, NR⁸R⁹, OR⁸, SR⁸, SSR⁸, CONR⁸R⁹, N₃, OP(O)OR⁸R⁹, PR⁸R⁹, oxo, an optionally substituted C₁₋₃₀ alkyl, an optionally substituted C₂₋₃₀ alkenyl, an optionally substituted C₆₋₂₀ aryl, an optionally substituted C₂₋₃₀ alkynyl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to membered heteroaryl or an optionally substituted 3 to 20 membered heterocycle, wherein R⁸ and R⁹ are each independently H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle.

Similarly, arylene, cycloalkylene, cycloalkenylene, cycloalkynylene, heteroarylene and heterocycylene groups are analogous to the above defined aryl, cycloalkyl, cycloalkenyl, cycloalkynyl, heteroaryl and heterocycyl groups, except they are bivalent.

In some embodiments m is 0.

In some embodiments, L³ is absent.

R² and R³ may independently be H or an optionally substituted C₁₋₁₂ alkyl. More preferably, R² and R³ are H or a C₁₋₆ alkyl, and even more preferably R² and R³ are H or a C₁₋₃ alkyl.

X¹ may be absent.

Accordingly, in some embodiments, the amphiphilic polymer chains are or comprise a plurality of polymers of formula IV:

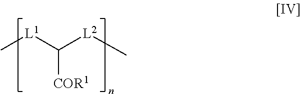

In some embodiments, L¹ has the following structure:

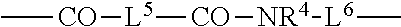

wherein L⁵ is an optionally substituted C₆₋₁₂ arylene or an optionally substituted C₅₋₁₀ heterocyclylene; and
L⁶ is an optionally substituted C₁₋₁₂ alkylene, an optionally substituted C₂₋₁₂ alkenylene or an optionally substituted C₂₋₁₂ alkynylene.

L⁵ may be an optionally substituted phenylene, and is preferably unsubstituted phenylene.

L⁶ may be an optionally substituted C₁₋₁₂ alkylene, preferably an optionally substituted C₂₋₆ alkylene, and more preferably —(CH₂)₄—.

R⁴ is preferably H.

L² may be NR⁴. R⁴ is preferably H.

Accordingly, in some embodiments, the polymer of formula IV has formula IVa:

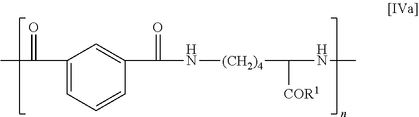

The amphiphilic polymer chains may have a number average molecular weight of at least 1 kDa, at least 2 kDa, at least 5 kDa, at least 10 kDa, at least 15 kDa, at least 20 kDa, at least 22 kDa, at least 24 kDa, at least 24.5 kDa or at least 24.8 kDa. The amphiphilic polymer chains may have a number average molecular weight of less than 250 kDa, less than 100 kDa, less than 75 kDa, less than 50 kDa, less than 40 kDa, less than 30 kDa, less than 28 kDa, less than 26 kDa, less than 25.5 kDa or less than 25 kDa. The amphiphilic polymer chains may have a number average molecular weight between 1 and 250 kDa, between 2 and 100 kDa, between 5 and 75 kDa, between 10 and 50 kDa or between 15 and 40 kDa, more preferably between 20 and 30 kDa, between 22 and 28 kDa, between 24 and 26 kDa or between 24.5 and 25.5 kDa, most preferably between 24.8 and 25 kDa.

n may be an integer of at least 1, at least 2, at least 3, at least 4, at least 5, at least 6, at least 7 or at least 8. n may be an integer of less than 50, less than 40, less than 30, less than 25, less than 20, less than 15, less than 12 or less than 10. n may be an integer between 1 and 50, between 2 and 40, between 3 and 30, between 4 and 25, between 5 and 20, between 6 and 15, between 7 and 12 or between 8 and 10.

Alternatively, n may be an integer of at least 5, at least 10, at least 15, at least 25, at least 50, at least 75, at least 90 or at least 95. n may be an integer of less than 5,000, less than 1,000, less than 500, less than 250, less than 150, less than 125, less than 110 or less than 105. n may be an integer between 5 and 5,000, between 10 and 1,000, between 15 and 500, between 25 and 250, between 50 and 150, between 75 and 125, between 90 and 110 or between 95 and 105. Typically, n may be an integer of between and 1,000, between 15 and 750, between 25 and 500, between 40 and 250, between 60 and 200, between 90 and 170, between 110 and 150, or between 120 and 140.

In further alternative embodiments, n may be an integer of at least 100, at least 250, at least 500, at least 750, at least 1,000, at least 1,250 or at least 1,500.

In some embodiments, m may be an integer which is at least 1. Accordingly, m may be an integer of at least 5, at least 10, at least 15, at least 20, at least 25, at least 30, at least 35, at least 40 or at least 45. m may be an integer of less than 1,000, less than 500, less than 250, less than 150, less than 100, less than 80, less than 70, less than 60 or less than 55. m may be an integer between 5 and 1,000, between 10 and 500, between 15 and 250, between 20 and 150, between 25 and 100, between 30 and 80, between 35 and 70, between 40 and 60 or between 45 and 55.

In embodiments where m is an integer which is at least 1, the ratio of n to m may be between 1:25 and 50:1, between 1:10 and 30:1, between 1:5 and 15:1, between 1:2 and 10:1, between 1:1 and 5:1 or between 1.5:1 and 3:1. In some embodiments, the ratio of n to m is 2:1.

It may be appreciated that controlling the ratio of n to m may control the hydrophilicity of the amphiphilic polymer.

L¹ may be an optionally substituted C₁₋₃₀ alkylene, an optionally substituted C₂₋₃₀ alkenylene or an optionally substituted C₂₋₃₀ alkynylene. More preferably, L¹ is an optionally substituted C₁₋₆ alkylene, an optionally substituted C₂₋₆ alkenylene or an optionally substituted C₂₋₆ alkynylene, and most preferably L¹ is a C₁₋₃ alkylene, a C₂₋₃ alkenylene or a C₂₋₃ alkynylene. Accordingly, L¹ may be —CH₂—.

L² may comprise one or more linker elements selected from the group consisting of CO, O, S and NR⁴. In some embodiments, L² may comprise or be one or two linker elements selected from CO and NR⁴. In alternative embodiments, L² may comprise or be one or more linker elements selected from the group consisting of CO, O and S, and more preferably L² may comprise one or two linker elements of CO and O. Accordingly, L² may be O, CO or COO.

L³ may comprise one or more linker elements selected from the group consisting of an optionally substituted C₁₋₃₀ alkylene, an optionally substituted C₂₋₃₀ alkenylene, an optionally substituted C₂₋₃₀ alkynylene, CO, O, S and NR⁴.

In some embodiments, L³ may be -L⁷-L⁸-L⁹-, wherein L⁷ and L⁹ are independently absent or an optionally substituted C₁₋₃₀ alkylene, an optionally substituted C₂₋₃₀ alkenylene or an optionally substituted C₂₋₃₀ alkynylene; and L⁸ may independently be absent or comprise one or more linker elements selected from the group consisting of CO, O, S and NR⁴, wherein at least one of L⁷ to L⁸ is present.

L⁷ and L⁹ may independently be an optionally substituted C₁₁₅ alkylene, an optionally substituted C₂₋₁₅ alkenylene or an optionally substituted C₂₋₁₅ alkynylene, more preferably L⁷ and L⁹ are independently an optionally substituted C₁₋₆ alkylene, an optionally substituted C₂₋₆ alkenylene or an optionally substituted C₂₋₆ alkynylene, and most preferably L⁷ and L⁹ are independently a C₁₋₃ alkylene, a C₂₋₃ alkenylene or a C₂₋₃ alkynylene. Accordingly, L⁷ and L⁹ may each be —CH₂CH₂—.

L⁸ may be CO, COO, O or S, and is preferably O or S, and more preferably is S.

L⁴ may comprise one or more linker elements selected from the group consisting of an optionally substituted C₁₋₃₀ alkylene, an optionally substituted C₂₋₃₀ alkenylene, an optionally substituted C₂₋₃₀ alkynylene, CO, O, S and NR⁴.

In some embodiments, L⁴ may be -L¹⁰-L¹¹-, wherein L¹⁰ is an optionally substituted C₁₋₃₀ alkylene, an optionally substituted C₂₋₃₀ alkenylene or an optionally substituted C₂₋₃₀ alkynylene; and L¹¹ may be absent or comprise one or more linker elements selected from the group consisting of CO, O, S and NR⁴.

L¹⁰ may be an optionally substituted C₁₋₃₀ alkylene, an optionally substituted C₂₋₃₀ alkenylene or an optionally substituted C₂₋₃₀ alkynylene, more preferably L¹⁰ is an optionally substituted C₁₀₋₁₅ alkylene, an optionally substituted C₁₀₋₁₅ alkenylene or an optionally substituted C₁₀₋₁₅ alkynylene, and most preferably L¹⁰ is a branched C₁₀₋₁₅ alkylene, a branched C₁₀₋₁₅ alkenylene or a branched C₁₀₋₁₅ alkynylene. Accordingly, L¹⁰ may be —CH₂CH((CH₂)₉CH₃)—.

L¹¹ may be O or S, and is preferably O.

R² may be H or an optionally substituted C₁₋₁₂ alkyl. More preferably, R² is H or an optionally substituted C₁₋₆ alkyl, and even more preferably R² is an optionally substituted C₁₋₃ alkyl. Accordingly, R² may be an optionally substituted methyl, optionally substituted ethyl or optionally substituted propyl. The alkyl may be substituted with a C₆₋₂₀ aryl or a 5 to 20 membered heteroaryl. The alkyl may be substituted with a phenyl. Accordingly, in some embodiments, R² is —CH₂—C₆H₅.

R³ may be H or an optionally substituted C₁₋₁₂ alkyl. More preferably, R³ is H or an optionally substituted C₁₋₆ alkyl, and even more preferably R³ is H or an optionally substituted C₁₋₃ alkyl.

X¹ may be O, S or NR⁵. X¹ is preferably O.

Accordingly, in some embodiments, the polymer may be polymer L₁₀₀F₅₀, as described in international patent publication no. WO 2021/260392 A1. The structure of L₁₀₀F₅₀ is provided below.

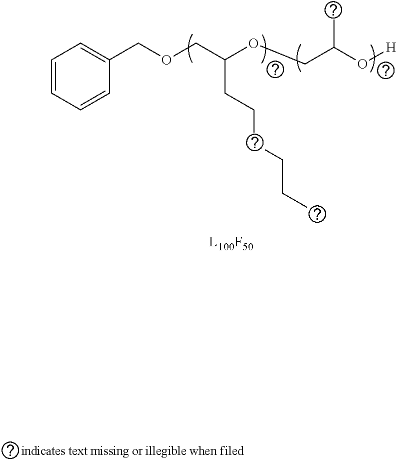

It will be appreciated that alternative multifunctional, membrane-permeabilizing multi-block polymers which fall within the scope of formula I may also be prepared using the methods described in WO 2021/260392 A1.

In some embodiments, at least 1% of the R¹ group in the polymers are OH, more preferably at least 5%, at least 10% or at least 15% of the R¹ group in the polymers are OH, and more preferably at least 20%, at least 22% or at least 24% of the R¹ group in the polymers are OH. In some embodiments, at least 25%, at least 30% or at least 35% of the R¹ group in the polymers are OH. In some embodiments, at least 50%, at least 60%, at least 70% or at least 80% of the R¹ group in the polymers are OH. In some embodiments, less than 99%, less than 95% or less than 90% of the R¹ group in the polymers are OH, more preferably less than 85% of the R¹ group in the polymers are OH. In some embodiments, less than 75%, less than 60%, less than 50% or less than 45% of the R¹ group in the polymers are OH. In some embodiments, less than 40%, less than 35%, less than 30%, less than 28% or less than 26% of the R¹ group in the polymers are OH. In some embodiments, between 1 and 99% of the R¹ group in the polymers are OH, more preferably between 5 and 85% of the R¹ group in the polymers are OH. In some embodiments, between 10 and 60% or between 15 and 40%, between and 35%, between 22 and 30% or between 24 and 26% of the R¹ group in the polymers are OH. In some embodiments, between 10 and 80% or between 15 and 70%, between 20 and 60%, between 30 and 50% or between 35 and 45% of the R¹ group in the polymers are OH. In some embodiments, between 50 and 92% or between 60 and 90%, between 70 and 88%, between 75 and 86% or between 80 and 84% of the R¹ group in the polymers are OH.

In some embodiments, at least 1%, at least 5% or at least 10% of the R¹ group in the polymers are NR⁶R⁷ or OR¹⁰, more preferably at least 15% of the R¹ group in the polymers are NR⁶R⁷ or OR¹⁰. In some embodiments, at least 25%, at least 50% or at least 60% of the R¹ group in the polymers are NR⁶R⁷ or OR¹⁰. In some embodiments, at least 65%, at least 70%, at least 72% or at least 74% of the R¹ group in the polymers are NR⁶R⁷ or OR¹⁰. In some embodiments, less than 99% of the R¹ group in the polymers are NR⁶R⁷ or OR¹⁰, more preferably less than 95%, less than 90% or less than 85% of the R¹ group in the polymers are NR⁶R⁷ or OR¹⁰, more preferably less than 80%, less than 78% or less than 76% of the R¹ group in the polymers are NR⁶R⁷ or OR¹⁰. In some embodiments, less than 70% or less than 65% of the R¹ group in the polymers are NR⁶R⁷ or OR¹⁰. In some embodiments, less than 60%, less than 40%, less than 30%, less than 25% or less than 20% of the R¹ group in the polymers are NR⁶R⁷ or OR¹⁰. In some embodiments, between 1 and 99%, between 5 and 98% or between 10 and 97% of the R¹ group in the polymers are NR⁶R⁷ or OR¹⁰, more preferably between 15 and 95% of the R¹ group in the polymers are NR⁶R⁷ or OR¹⁰. In some embodiments, between 40 and 90%, between 60 and 85%, between 65 and 80%, between 70 and 78% or between 74 and 76% of the R¹ group in the polymers are NR⁶R⁷ or OR¹⁰. In some embodiments, between 20 and 80%, between 30 and 75%, between 40 and 70%, between 50 and 67.5%, between 55 and 65% or between 57.5 and 62.5% of the R¹ group in the polymers are NR⁶R⁷ or OR¹⁰. In some embodiments, between 2.5 and 60%, between 5 and 50%, between 7.5 and 40%, between 10 and 30%, between 12.5 and 25% or between 15 and 20% of the R¹ group in the polymers are NR⁶R⁷ or OR¹⁰.

In alternative embodiments, at least 30% of the R¹ group in the polymers are OH, at least 40%, at least 50%, at least 60%, at least 70%, at least 80% or at least 90% of the R¹ group in the polymers are OH. In some embodiments, 100% of the R¹ group in the polymers may be OH. This embodiment is preferred when m is an integer of at least 1.

It may be appreciated that controlling the percentage of R¹ groups which are OH may control the hydrophilicity of the polymer.

In some embodiments, the polymer is a polymer of formula IVa and 100% of the R¹ group in the polymers are OH.

R⁶ and R⁷ may independently each be H, an optionally substituted C₁₋₂₀ alkyl, an optionally substituted C₂₋₂₀ alkenyl or an optionally substituted C₂₋₂₀ alkynyl. More preferably, R⁶ and R⁷ may independently each be H, an optionally substituted C₁₋₁₂ alkyl, an optionally substituted C₂₋₁₂ alkenyl or an optionally substituted C₂₋₁₂ alkynyl. R⁶ may be H.

In one embodiment, R⁷ may be an optionally substituted C₁₋₆ alkyl, an optionally substituted C₂₋₆ alkenyl or an optionally substituted C₂₋₆ alkynyl. The alkyl, alkenyl or alkynyl may be unsubstituted or substituted with one or more substituents, as defined above. In some embodiments, the alkyl, alkenyl or alkynyl is unsubstituted or substituted with one or more of halogen, COOH, oxo, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to membered heteroaryl or an optionally substituted 3 to 20 membered heterocycle. Preferably, the alkyl, alkenyl or alkynyl is substituted with one or more of halogen, COOH, oxo, a C₆₋₂₀ aryl, a C₃₋₂₀ cycloalkyl, a C₃₋₂₀ cycloalkenyl, a C₃₋₂₀ cycloalkynyl, a 5 to 20 membered heteroaryl or a 3 to 20 membered heterocycle. In some embodiments, the alkyl is substituted with COOH and optionally further substituted with phenyl or 1H-indolyl.

Accordingly, R⁷ may be

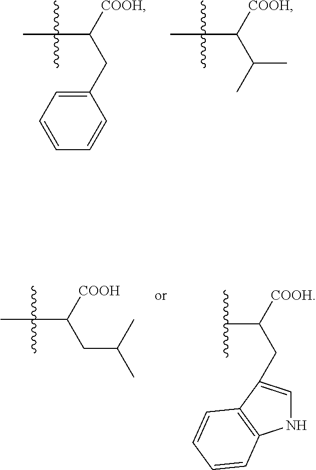

More preferably, R⁷ is

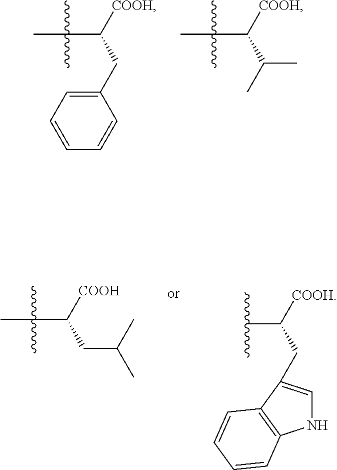

In some embodiments, R⁷ is

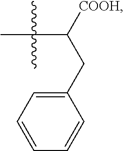 [SMILES: *C(Cc1ccccc1)C(=O)O]

and more preferably

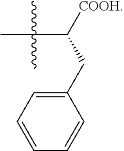 [SMILES: *[C@@H](Cc1ccccc1)C(=O)O]

Accordingly, the polymers may comprise a phenylalanine side chain.

In some embodiments, the polymer is a polymer of formula IVa, wherein at least one R¹ group is NR⁶R⁷, R⁶ is H and R⁷ is

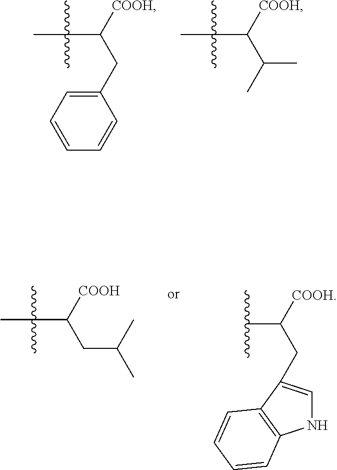

More preferably, R⁷ is

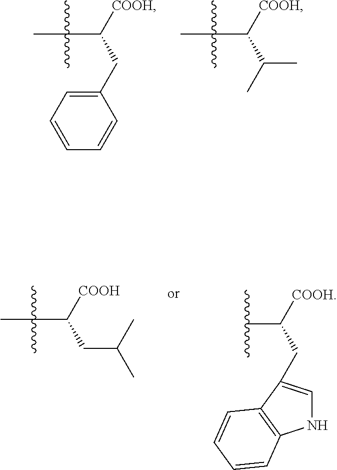

most preferred embodiment, R⁷ is

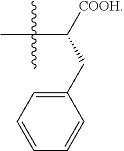 [SMILES: *[C@@H](Cc1ccccc1)C(=O)O]

In an alternative embodiment, R⁷ is an optionally substituted C₃₋₂₅ alkyl, an optionally substituted C₃₋₂₅ alkenyl or an optionally substituted C₃₋₂₅ alkynyl. More preferably, R⁷ is an optionally substituted C₅₋₂₀ alkyl, an optionally substituted C₅₋₂₀ alkenyl or an optionally substituted C₅₋₂₀ alkynyl. The alkyl, alkenyl or alkynyl may be unsubstituted or substituted with one or more substituents, as defined above. In some embodiments, the alkyl, alkenyl or alkynyl is unsubstituted or substituted with one or more of halogen, COOH, oxo, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle. In some embodiments, R⁷ is a C₅₋₂₀ unsubstituted alkyl. In alternative embodiments, R⁷ is a C₅₋₂₀ substituted alkyl. The alkyl may be substituted with COOH.

Accordingly, R⁷ may be —(CH₂)₆CH₃, —(CH₂)₉CH₃, —(CH₂)₁₃CH₃, —(CH₂)₁₇CH₃ or —(CH₂)₁₁COOH.

In some embodiments, the polymer is a polymer of formula IVa, wherein at least one R¹ group is NR⁶R⁷, R⁶ is H and R⁷ is —(CH₂)₆CH₃, —(CH₂)₉CH₃, —(CH₂)₁₃CH₃, —(CH₂)₁₇CH₃ or —(CH₂)₁₁COOH.

These polymers are anionic amphiphilic polycarboxylates containing hydrophobic pendant chains and ionizable carboxylic acid groups. They are designed to mimic the structure and pH-responsive membrane-permeabilizing activity of the anionic fusogenic peptides in the hemagglutinin spikes of influenza virus. These biomimetic polymers are simple to synthesize and display pH-responsive membrane activity due to their ability to change in conformation, from coiled to globular, when pH is reduced below their pKa.

The plurality of polymer chains may be conjugated to the lipid structure or may not be conjugated to the lipid structure. In a preferred embodiment, the plurality of polymer chains are not conjugated to the lipid structure. The plurality of polymer chains may interact with the lipid structure due to electrostatic and/or hydrophobic forces.

The sub-micron particle may comprise at least 100 or at least 500 polymer chains, more preferably at least 1,000, at least 2,000, at least 4,000, at least 6,000, at least 8,000 or at least 10,000 polymer chains. The sub-micron particle may comprise between 50 and 1,000,000 polymer chains, or between 100 and 100,000 or between 500 and 50,000 polymer chains, more preferably between 1,000 and 40,000, between 2,000 and 30,000, between 4,000 and 25,000, between 6,000 and 20,000, between 8,000 and 15,000 or between 10,000 and 13,000 polymer chains.

The sub-micron particle may comprise the polymer chains at a concentration of at least 0.01 mg/ml, at least 0.05 mg/ml, at least 0.1 mg/ml, at least 0.2 mg/ml, at least 0.25 mg/ml, at least 0.3 mg/ml, at least 0.5 mg/ml, at least 0.75 mg/ml or at least 1 mg/ml. The sub-micron particle may comprise the polymer chains at a concentration of between 0.01 and 25 mg/ml, between 0.05 and 10 mg/ml, between 0.1 and 5 mg/ml, between 0.2 and 2.5 mg/ml, between 0.25 and 2 mg/ml, between 0.3 and 1.5 mg/ml or between 0.5 and 1 mg/ml.

In some embodiments, the outer surface of the lipid structure is fully saturated with the plurality of polymer chains.

It may be appreciated that the sub-micron particle may comprise one or more lipid structures. More preferably, the sub-micron particle may comprise a single lipid structure. Accordingly, the number of polymer chains given above may be viewed as the number of polymer chains per lipid structure, and preferably the number of polymer chains per lipid nanoparticle.

The sub-micron particle may be negatively charged at a pH of at least 5 at 20° C., at least 6 at 20° C., at least 6.5 at 20° C. or at least 7 at 20° C. The sub-micron particle may be negatively charged at a pH of between 5 and 9 at 20° C., between 6 and 8 at 20° C. or between 6.5 and 7.5 at 20° C.

The sub-micron particle may comprise a second payload molecule. The second payload molecule is preferably encapsulated in the lipid structure. Alternatively or additionally, the second payload molecule may be covalently conjugated and/or physically attached to the outer surface of the lipid structure. The first and second payload molecules may be different types, species or structures.

The second payload molecule may preferably be an active pharmaceutical ingredient (API) or component thereof, or facilitator or enabler thereof, such as an enzyme (e.g. CRISPR-Cas9 etc.). The second payload molecule may be a hydrophobic or hydrophilic second payload molecule. The second payload molecule may be a macromolecule or a small molecule.

It may be appreciated that a small molecule could be considered to be a molecule with a molecular weight of less than 900 daltons. In some embodiments, a small molecule may have a molecular weight of less than 800 daltons, less than 700 daltons, less than 600 daltons, less than 500 daltons or less than 400 daltons.

Any small molecule drugs, defined as a chemical entity of low molecular weight, may be used as the second payload molecule. For example, anticancer and anti-inflammatory drugs, such as steroids may be used. Additional examples may include Ruxolitinib and other Janus Kinase inhibitors, Doxorubicin, Tofacitinib, Curcumin, Paclitaxel, Cisplatin, Buprenorphine, Aspirin, Ibuprofen, or Naproxen etc.

Advantageously, the inventors have shown that the sub-micron particle can co-deliver a macromolecule, such as a nucleic acid, and a second payload molecule.

The sub-micron particle may further comprise at least one stabilizing molecule. The at least one stabilizing molecule may be encapsulated in the lipid structure. Alternatively, or additionally, the at least one stabilizing molecule may be disposed outside the lipid structure. In some embodiments, the at least one stabilizing molecule outside the lipid structure may be covalently conjugated and/or physically attached to the outer surface of the lipid structure.

The or each stabilizing molecules may be a carbohydrate and/or a polyol.

The carbohydrate may be referred to as a sugar. The carbohydrate may be a monosaccharide, which may be selected from a group consisting of: glucose; galactose; fructose; mannose; and xylose or a pharmaceutically acceptable complex, salt, solvate, tautomeric form, stereoisomer or polymorphic form thereof. Alternatively, the carbohydrate may be a disaccharide, which may be selected from a group consisting of: trehalose; sucrose; lactose; maltose; isomaltose; lactitol; lactulose; mannobiose; and isomalt or a pharmaceutically acceptable complex, salt, solvate, tautomeric form, stereoisomer or polymorphic form thereof. In a further alternative, the carbohydrate may be a trisaccharide, which may be selected from a group consisting of: nigerotriose; maltotriose; melezitose; maltotriulose; raffinose; and kestose or a pharmaceutically acceptable complex, salt, solvate, tautomeric form, stereoisomer or polymorphic form thereof. In a further alternative, the carbohydrate may be a polysaccharide, which may be selected from the group consisting of: dextran; amylose; amylopectin; glycogen; galactogen; inulin; callose; cellulose; chitosan; and chitin or a pharmaceutically acceptable complex, salt, solvate, tautomeric form, stereoisomer or polymorphic form thereof.

The carbohydrate may be a polyol, which may be selected from a group consisting of: sorbitol; mannitol; glycerol; alpha-D-glucopyranosyl-1-6-sorbitol; alpha-D-glucopyranosyl-1-6-mannitol; a malto-oligosaccharide; a hydrogenated maltooligosaccharide, starch and cellulose or a pharmaceutically acceptable complex, salt, solvate, tautomeric form, stereoisomer or polymorphic form thereof.

Alternatively or additionally, the polyol may be an oligomer comprising a plurality of hydroxyl groups; a polymer comprising a plurality of hydroxyl groups or a pharmaceutically acceptable complex, salt, solvate, tautomeric form, stereoisomer or polymorphic form thereof.

The at least one stabilizing molecule may comprise at least two different stabilizing molecules. Each stabilizing molecule may be a carbohydrate. In one embodiment, a first stabilizing molecule may be a disaccharide (e.g., trehalose) and a second stabilizing molecule may be a polysaccharide (e.g., dextran).

In some embodiments, the carbohydrate is a disaccharide, and most preferably trehalose, or a pharmaceutically acceptable complex, salt, solvate, tautomeric form, stereoisomer or polymorphic form thereof. The trehalose may be synthetic trehalose or natural trehalose.

Advantageously, the inventors have surprisingly demonstrated that when the sub-micron particle comprises trehalose, then it can stabilise a macromolecule such as a nucleic acid, both lyophilized and in aqueous solution, for a prolonged period of time at room temperature. Additionally, storage at 40° C. is also enhanced. This was totally unexpected.

Preferably, the sub-micron particle of the first aspect is thermally stabilized.

It will be appreciated that the expression “thermal stabilization” or “thermally stabilized” can mean that the sub-micron particle substantially retains its biological activity (e.g., it elicits an immune response and/or protein expression in a subject administered therewith) when stored at certain temperatures for a period of time. Although the inventors do not wish to be bound by any hypothesis, they believe that the thermal stabilization effects may be realised by stabilizing the lipid structure in the formulation, for example by preventing or reducing its aggregation; by minimizing the leakage of the payload; by stabilizing the payload molecule (preferably RNA) per se; and/or by stabilizing the sub-micron particle to have improved colloidal stability. Whether or not the functional activity of the payload molecule (preferably RNA) is retained, or the extent thereof, can be determined for example by detecting the presence of immunospecific antibodies (e.g., IgG) raised against the antigen of interest encoded by the RNA construct and/or detecting the expression of a protein of interest.

Preferably, the sub-micron particle is thermally stabilised following storage at a temperature of −100° C. and above, −80° C. and above, −60° C. and above, −40° C. and above or −20° C. and above, more preferably −15° C. and above, and most preferably −10° C. and above. Preferably, the sub-micron particle is thermally stabilised following storage at a temperature of −5° C. and above, more preferably 0° C. and above, and most preferably 1° C. and above. Most preferably, the sub-micron particle is thermally stabilised following storage at a temperature of 2° C. and above, more preferably 3° C. and above, and most preferably 4° C. and above. Even more preferably, the sub-micron particle is thermally stabilised following storage at a temperature of 5° C. and above, more preferably 6° C. and above, and most preferably 7° C. and above.

The sub-micron particle may be thermally stabilised following storage at a temperature of less than 100° C., less than 80° C., less than 60° C., less than 50° C., less than 40° C., less than 35° C., or less than 30° C. The sub-micron particle may be thermally stabilised following storage at a temperature of less than 25° C., less than 20° C., or less than 15° C. The sub-micron particle may be thermally stabilised following storage at a temperature of less than 10° C., less than 8° C., or less than 7° C.

The sub-micron particle may be thermally stabilised following storage at a temperature of between −100° C. and 100° C., between −80° C. and 90° C., between −60° C. and 80° C., between −40° C. and 70° C., between −20° C. and 60° C., between −20° C. and 50° C., between −20° C. and 40° C., between −20° C. and 35° C., between −20° C. and 30° C., between −15° C. and 25° C., or between −10° C. and 20° C. The sub-micron particle may be thermally stabilised following storage at a temperature of between −5° C. and 15° C., between 0° C. and 10° C., between 1° C. and 9° C., or between 2° C. and 8° C.

The sub-micron particle may comprise at least one targeting ligand or moiety. The at least one targeting ligand or moiety may be disposed on an outer surface of the submicron particle. Accordingly, the at least one targeting ligand or moiety may be disposed on an outer surface of the lipid structure and/or on the amphiphilic polymer chains.

The at least one targeting ligand or moiety may be or comprise at least one of a peptide, a protein, an aptamer, a carbohydrate, an oligosaccharide, a folic acid or folate, and antibody or an antigen binding fragment thereof, a vitamin or a derivative thereof. The peptide may be a G protein-coupled receptor (GCR), Arg-Gly-Asp (RGD), or a derivative thereof. The proteins may be a lectin, a transferrin, or a derivative thereof. The aptamers may be an RNA aptamer against HIV glycoprotein, or a derivative thereof. The carbohydrates may be as defined above. In particular, the carbohydrate may be mannose, glucose, galactose, or a derivative thereof. The antibody may be monoclonal or polyclonal. The antibody may be an anti-Her2 antibody, an anti-EGFR antibody, or a derivative thereof. The vitamin may be vitamin D.

The inventors have devised a novel method for producing the payload delivery systems, and sub-micron particles of the invention.

Thus, in accordance with a second aspect, there is provided a method of producing a sub-micron particle, the method comprising combining a first payload molecule, a lipid structure and a plurality of amphiphilic polymer chains to produce the sub-micron particle, wherein the first payload molecule is a macromolecule, optionally a nucleic acid, and the hydrophilicity of the amphiphilic polymer chains changes in response to an external stimulus.

Preferably, the method of the second aspect produces the sub-micron particle of the first aspect.

Advantageously, and preferably, the method of the second aspect can be used to modify the existing lipid structure, including lipid nanoparticle and liposome systems.

The first payload molecule, the lipid structure (comprising a plurality of lipids) and the plurality of amphiphilic polymer chains may be as defined in relation to the first aspect.

The first payload molecule may be covalently conjugated and/or physically attached to the outer surface of the lipid structure. Preferably, the first payload molecule is encapsulated in the lipid structure.

The method may therefore comprise contacting the first payload molecule and a plurality of lipids to produce a lipid structure encapsulating the first payload molecule. The method may subsequently comprise contacting the lipid structure and the plurality of amphiphilic polymer chains to produce the sub-micron particle.

In some embodiments, the method may comprise contacting the first payload molecule, a second payload molecule and a plurality of lipids to produce a lipid structure encapsulating, or being covalently conjugated and/or physically attached onto its outer surface with, the first and second payload molecules; and subsequently contacting the lipid structure and the plurality of amphiphilic polymer chains to produce the sub-micron particle.

The second payload molecule is preferably encapsulated in the lipid structure. Alternatively, the second payload molecule may be covalently conjugated and/or physically attached to the outer surface of the lipid structure. The second payload molecule is preferably different to the first payload molecule, i.e. in terms of being a different type, species or chemical structure.

In some embodiments, the method may comprise contacting the first payload molecule, at least one stabilizing molecule and a plurality of lipids to produce a lipid structure containing the first payload molecule and the at least one stabilizing molecule; and subsequently contacting the lipid structure and the plurality of amphiphilic polymer chains to produce the sub-micron particle. The first payload molecule is preferably encapsulated in the lipid structure. Alternatively, the first payload molecule may be covalently conjugated and/or physically attached to the outer surface of the lipid structure. The at least one stabilizing molecule may be encapsulated in the lipid structure. Alternatively, or additionally, the at least one stabilizing molecule may be disposed outside the lipid structure.

In some embodiments, the method may comprise contacting the first payload molecule, the second payload molecules, at least one stabilizing molecule and a plurality of lipids to produce a lipid structure containing the first and second payload molecules and the at least one stabilizing molecule; and subsequently contacting the lipid structure and the plurality of amphiphilic polymer chains to produce the sub-micron particle. The first and second payload molecules are preferably encapsulated in the lipid structure. Alternatively, the first and/or second payload molecules may be covalently conjugated and/or physically attached to the outer surface of the lipid structure. The at least one stabilizing molecule may be encapsulated in the lipid structure. Alternatively, or additionally, the at least one stabilizing molecule may be disposed outside the lipid structure.

Contacting the first payload molecule and the plurality of lipids to form a lipid structure may comprise contacting the first payload molecule and a lipid film comprising the plurality of lipids in solution and extruding the solution to produce the lipid structure encapsulating the first payload molecule. It will be appreciated that in embodiments where they are present, the second payload molecule and the/or the at least one stabilizing molecule may also contact the first payload and lipid film in solution, and may subsequently be encapsulated in the lipid structure. The solution may comprise water. The solution may comprise a buffer. Preferably, the solution is substantially RNAse-free. The solution may comprise disodium hydrogen phosphate and/or sodium chloride. In some embodiments, phosphate-buffered saline (PBS), and more preferably Dulbecco's phosphate-buffered saline (DPBS) is used.

The method may comprise extruding the solution through a membrane. The membrane may have a size of between 0.005 and 50 μm, between 0.01 and 10 μm, between 0.05 and 5 μm, between 0.1 and 3 μm, or between 0.15 and 0.25 μm. The method may comprise extruding the solution between 1 and 10,000 times, between and 1,000 times, between 10 and 500 times, between 15 and 250 times, between 20 and 100 times, between 25 and 50 times or between 28 and 35 times.

The method may comprise contacting the lipid structure and the plurality of amphiphilic polymer chains in solution. The solution may comprise water. The solution may comprise a buffer. Preferably, the solution is substantially RNAse-free. The solution may comprise disodium hydrogen phosphate and/or sodium chloride. In some embodiments, phosphate-buffered saline (PBS), and more preferably Dulbecco's phosphate-buffered saline (DPBS) is used.

The method may comprise contacting the lipid structure and the plurality of amphiphilic polymer chains for at least 30 minutes, for at least 1 hour, for at least 2 hours, at least 4 hours, at least 6 hours, at least 8 hours or at least 12 hours.

The method may comprise removing excess polymers chains. The excess polymer chains may be removed using dialysis, centrifugation or ultracentrifugation.

The inventors believe that that sub-micron particles of the invention are novel per se.

Hence, in accordance with a third aspect, there is provided a sub-micron particle obtained or obtainable by the method of the second aspect.

In accordance with a fourth aspect, there is provided a composition comprising a plurality of sub-micron particles of the first or third aspect.

In some embodiments, the composition is a pharmaceutical composition and comprises a pharmaceutically acceptable vehicle.

The composition may comprise a solvent. The solvent may comprise a buffer.

Preferably, the solvent is substantially RNAse-free. It is preferred to use a buffer or saline solution, such as Dulbecco's phosphate buffered saline (DPBS), HEPES buffered saline, Tris buffered saline or other such cell culture compatible buffers. This is because the ions provide additional stability to the composition.

The sub-micron particle may be present at a concentration of between 1×10³ and 1×10²⁰ particles/ml, between 1×10⁵ and 1×10¹⁵ particles/ml, between 1×10⁷ and 1×10¹¹ particles/ml, between 1×10⁸ and 1×10¹⁰ particles/ml, between 1×10⁹ and 5×10⁹ particles/ml, between 2×10⁹ and 4×10⁹ particles/ml, between 2.5×10⁹ and 3.5×10⁹ particles/ml or between 2.8×10⁹ and 3×10⁹ particles/ml.

In embodiments where the sub-micron particle comprises at least one stabilizing molecule, the at least one stabilizing molecule may be present at a concentration of between 1 and 5000 mg/ml, between 10 and 4000 mg/ml, between 20 and 3000 mg/ml, between 30 and 2000 mg/ml, between 40 and 1000 mg/ml, between 50 and 750 mg/ml, between 60 and 500 mg/ml, between 80 and 400 mg/ml, between 100 and 350 mg/ml or between 200 and 300 mg/ml.

In embodiments where the sub-micron particle comprises at least one stabilizing molecule, the at least one stabilizing molecule may be present at a concentration of between 10 and 900 mg/ml, between 20 and 800 mg/ml, between 30 and 700 mg/ml, between 40 and 600 mg/ml, between 50 and 500 mg/ml, between 60 and 450 mg/ml, between 80 and 400 mg/ml, between 100 and 350 mg/ml or between 200 and 300 mg/ml.

Alternatively, the composition of the fourth aspect may not comprise a solvent. Accordingly, the composition may be freeze-dried, vacuum dried, air-dried or spray dried. More preferably, the composition may be freeze-dried.

In a fifth aspect, there is provided a method of preparing a pharmaceutical composition, the method comprising contacting a plurality of sub-micron particles of the first or third aspect with a pharmaceutically acceptable vehicle. In a sixth aspect, there is provided the sub-micron particle of the first or third aspect, or the composition of the fourth aspect, for use as a medicament.

In a seventh aspect, there is provided a method of treatment, the method comprising administering, or having administered, to a subject in need thereof, a therapeutic amount of the sub-micron particle of the first or third aspect, or the composition of the fourth aspect.

In an eighth aspect, there is provided a vaccine composition comprising the sub-micron particle of the first or third aspect, or the composition of the fourth aspect.

The vaccine may comprise a suitable adjuvant.

In a ninth aspect, there is provided the sub-micron particle of the first or third aspect, the composition of the fourth aspect or the vaccine of the eighth aspect, for use in stimulating an immune response in a subject.

The immune response may be stimulated against a protozoa, bacterium, virus, fungus or cancer.

In a tenth aspect of the invention, there is provided a method of vaccinating a subject, the method comprising administering, or having administered, to a subject in need thereof, a therapeutic amount of the sub-micron particle of the first or third aspect, the composition of the fourth aspect or the vaccine of the eighth aspect.

The sub-micron particle, the composition or the vaccine of the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The sub-micron particle, the composition or the vaccine of the invention may also be incorporated within a slow- or delayed-release device. Such devices may, for example, be inserted on or under the skin, and the medicament may be released over weeks or even months. The device may be located at least adjacent the treatment site.

In a preferred embodiment, however, medicaments according to the invention may be administered to a subject by injection into the blood stream, muscle, skin or directly into a site requiring treatment. Injections may be intravenous (bolus or infusion), subcutaneous (bolus or infusion), intradermal (bolus or infusion), intramuscular (bolus or infusion), intrathecal (bolus or infusion), epidural (bolus or infusion) or intraperitoneal (bolus or infusion).

It will be appreciated that the amount of sub-micron particle, the composition or the vaccine that is required is determined by its biological activity and bioavailability, which in turn depends on the mode of administration, the physiochemical properties of the sub-micron particle, the composition or the vaccine and whether it is being used as a monotherapy or in a combined therapy.

The frequency of administration will also be influenced by the half-life of the active agent within the subject being treated. Optimal dosages to be administered may be determined by those skilled in the art, and will vary with the sub-micron particle, the composition or the vaccine in use, the strength of the composition, the mode of administration, and the type of treatment. Additional factors depending on the particular subject being treated will result in a need to adjust dosages, including subject age, weight, gender, diet, and time of administration.

The required dose may depend upon a number of factors including, but not limited to, the active agent being administered, the disease being treated and/or vaccinated against, the subject being treated, etc.

Generally, a dose of between 0.001 μg/kg of body weight and 10 mg/kg of body weight, or between 0.01 μg/kg of body weight and 1 mg/kg of body weight, of the sub-micron particle, the composition or the vaccine of the invention may be used, depending upon the active agent used. A dose may be understood to relate to the quantity of the payload molecule(s) which is delivered.

Doses may be given as a single administration (e.g., a single injection). Alternatively, the sub-micron particle, the composition or the vaccine may require more than one administration. As an example, the sub-micron particle, the composition or the vaccine may be administered as two or more doses of between 0.07 g and 700 mg (i.e., assuming a body weight of 70 kg). Alternatively, a slow-release device may be used to provide optimal doses of the sub-micron particle, the composition or the vaccine according to the invention to a patient without the need to administer repeated doses. Routes of administration may incorporate intravenous, intradermal subcutaneous, intramuscular, intrathecal, epidural or intraperitoneal routes of injection.

Known procedures, such as those conventionally employed by the pharmaceutical industry (e.g., in vivo experimentation, clinical trials, etc.), may be used to form specific formulations of the sub-micron particle, the composition or vaccine according to the invention and precise therapeutic regimes (such as doses of the agents and the frequency of administration).

A “subject” may be a vertebrate, mammal, or domestic animal. Hence, compositions and medicaments according to the invention may be used to treat any mammal, for example livestock (e.g., a horse), pets, or may be used in other veterinary applications.

Most preferably, however, the subject is a human being.

A “therapeutically effective amount” of the sub-micron particle, the composition or the vaccine is any amount which, when administered to a subject, is the amount of the aforementioned that is needed to produce a therapeutic effect.

For example, a therapeutically effective amount of the sub-micron particle, the composition and the vaccine of the invention may comprise from about 0.001 mg to about 800 mg of the payload molecule, and preferably from about 0.01 mg to about 500 mg of the payload molecule.

A “pharmaceutically acceptable vehicle” as referred to herein, is any known compound or combination of known compounds that are known to those skilled in the art to be useful in formulating compositions.

In one embodiment, the pharmaceutically acceptable vehicle may be a solid, and the composition may be in the form of a powder, a capsule or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent (e.g., sub-micron particle of the invention) may be mixed with a vehicle having the necessary compression properties in suitable proportions and compacted in the shape and size desired. The pharmaceutical vehicle may be a gel and the composition may be in the form of a cream or the like.

Alternatively, the pharmaceutical vehicle may be a liquid, and the composition is in the form of a solution. Liquid vehicles are used in preparing solutions, suspensions, emulsions, syrups, elixirs and pressurized compositions. The sub-micron particle according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant. Liquid pharmaceutical compositions, which are sterile solutions or suspensions, can be utilized by, for example, intramuscular, intrathecal, epidural, intraperitoneal, intravenous and subcutaneous injection. The sub-micron particle of the invention may be prepared as any appropriate sterile injectable medium.

The sub-micron particle may be administered by inhalation. For instance, the sub-micron particle may be provided in the form of an aerosol.

The sub-micron particle and/or the composition of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The sub-micron particle of the invention and/or the composition according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

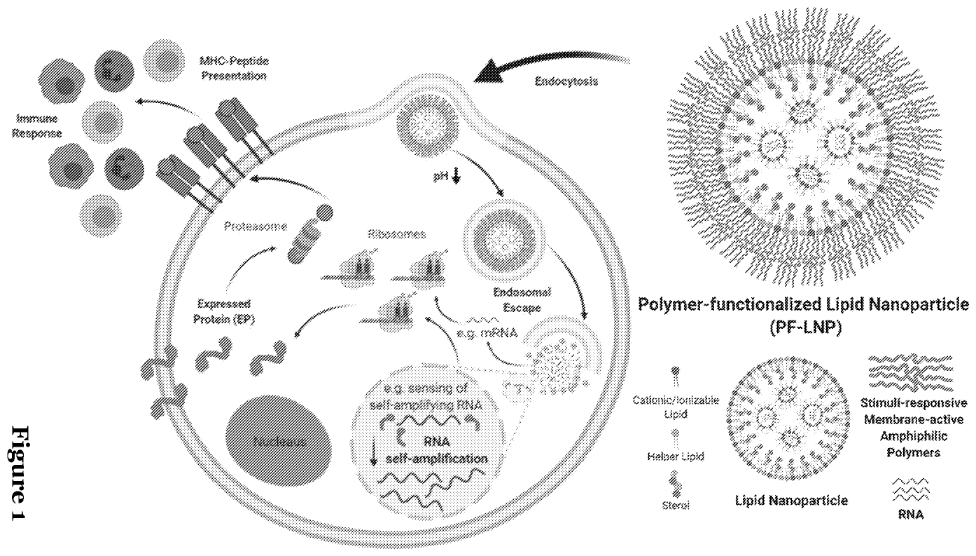

FIG. 1 is a schematic illustration of the polymer-functionalized lipid nanoparticle (PF-LNP) and the PF-LNP mediated intracellular delivery of RNA including RNA including messenger RNA (mRNA) or self-amplifying RNA (saRNA);

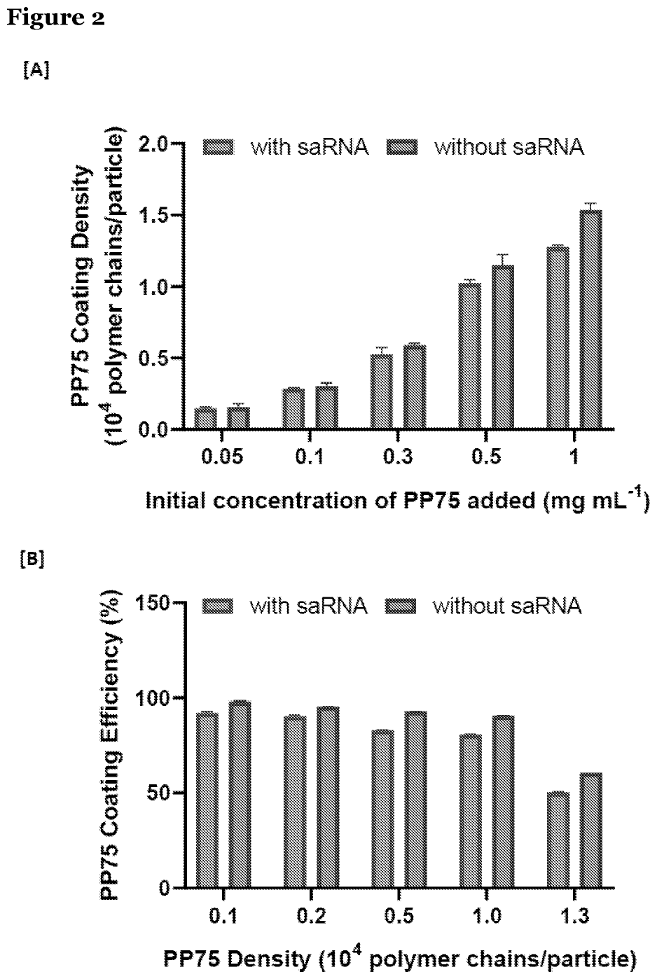

FIG. 2 is a graph showing the coating density of PP75, an anionic, viral-peptide-mimicking, membrane-permeabilizing pseudopeptidic polymer, when different concentrations of PP75 are added to the LNPs (DOTAP/DOPE weight ratio=1:1, 40 wt % cholesterol), after dialysis to remove excess polymer [A], the coating efficiency of PP75 based on density on the surface of the LNPs [B]. Mean±SD (n=3);

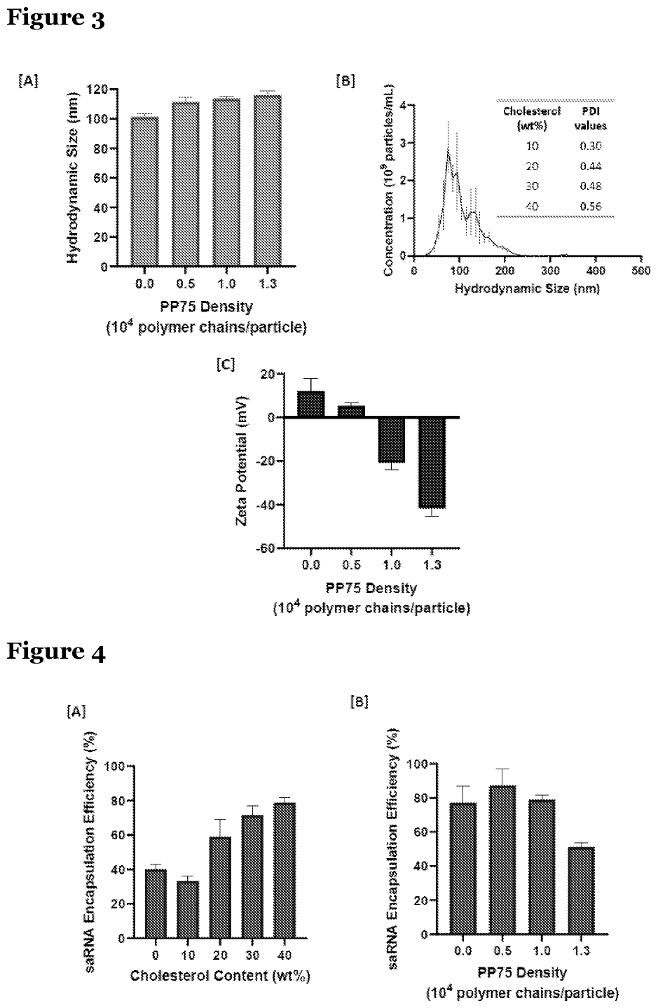

FIG. 3 shows the physiochemical characterization of PF-LNPs (DOTAP/DOPE=1:1, 40 wt % cholesterol and varying PP75 polymer coating densities) at pH 7.4: mean hydrodynamic diameters [A], Nanoparticle Tracking Analysis (NTA) measurement of size distribution, particle concentration and PDI values [B], and zeta potential measurements [C]. Mean±SD (n=3);

FIG. 4 shows the effect of cholesterol content (at a fixed PP75 density of 1.0×10⁴ polymer chains/particle) [A] and polymer coating density (at a fixed cholesterol content of 40 wt %) [B] on saRNA encapsulation efficiency in PF-LNPs (DOTAP/DOPE=1:1), as determined by RiboGreen™ Assay. Mean±SD (n=3);

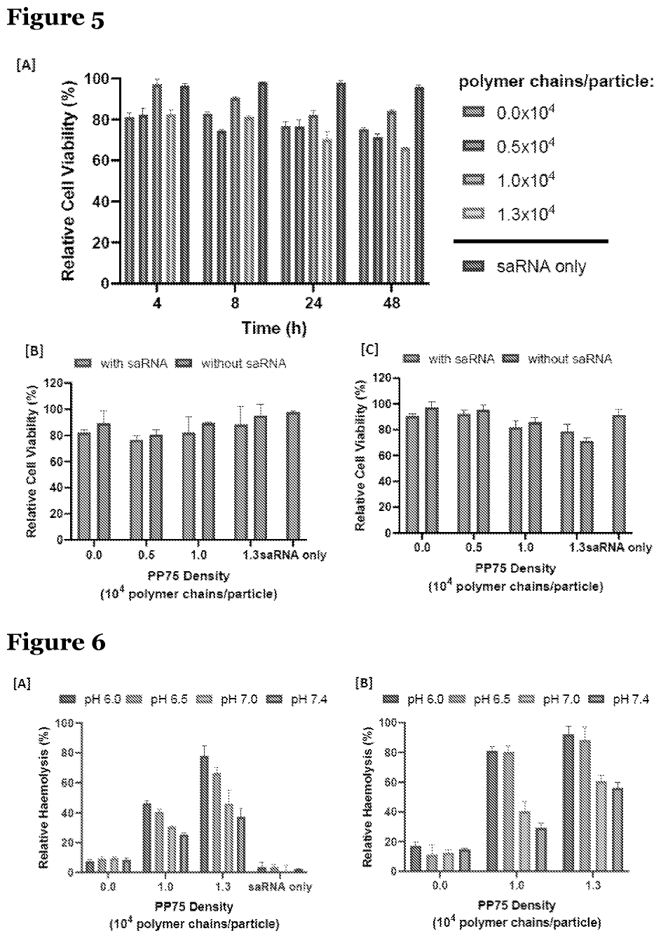

FIG. 5 shows relative Hela cell viability after 4, 8, 24 and 48 h of incubation, respectively, with saRNA-encapsulated PF-LNPs (DOTAP/DOPE=1:1, 40 wt % cholesterol and various PP75 coating densities) [A], as determined by Alamar Blue assay. Relative HeLa cell viability after 24 h of incubation with PF-LNPs (DOTAP/DOPE=1:1, 40 wt % cholesterol and various PP75 coating densities) with and without encapsulated saRNA [B], as determined by Alamar Blue assay. Relative HeLa cell viability after 24 h of incubation with PF-LNPs (DOTAP/DOPE=1:1, 40 wt % cholesterol and various PP75 coating densities) with and without encapsulating saRNA, as determined by LDH assay [C]. N/P ratio=1. Mean±S.D. (n=3);

FIG. 6 shows relative hemolysis of red blood cells (RBCs) after 1 h of incubation with PF-LNPs (DOTAP/DOPE=1:1, 40 wt % cholesterol, and various PP75 coating densities) at different pH values, with saRNA [A], and without saRNA [B]. N/P ratio=1. Mean±S.D. (n=3);

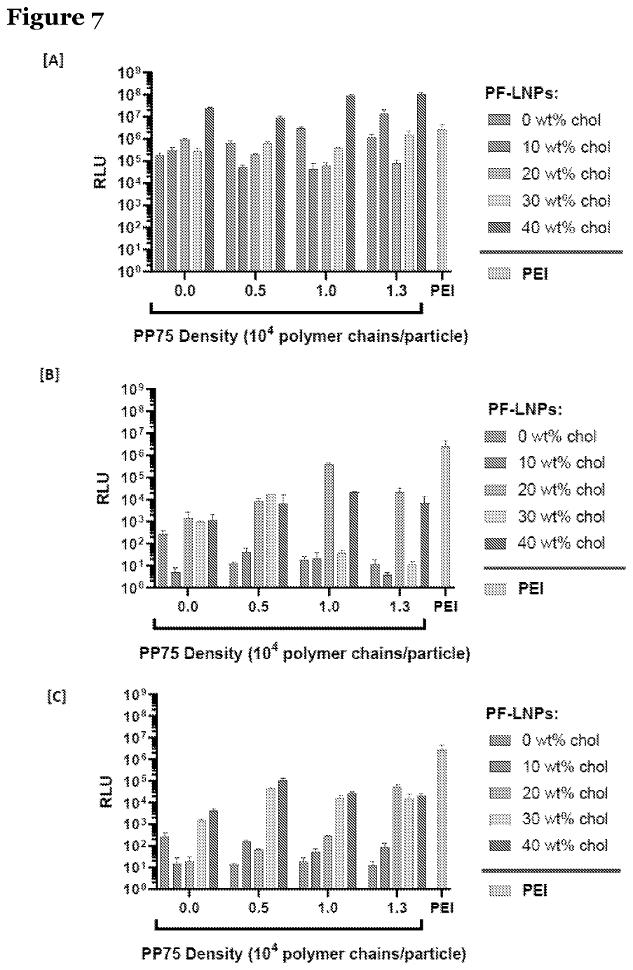

FIG. 7 shows HEK-293 cell transfection after 4 h of incubation with PF-LNPs consisting of DOTAP/DOPE at the weight ratio of 1:1 [A], 1:2 [B] and 1:4 [C], cholesterol at various percentages, and PP75 at various coating densities (N/P ratio=1, and PEI as a control), as determined by Firefly Luciferase (fLuc) assay. Luciferase expression was evaluated 24 h after transfection, expressed as relative light units (RLU). Mean±SD (n=3);

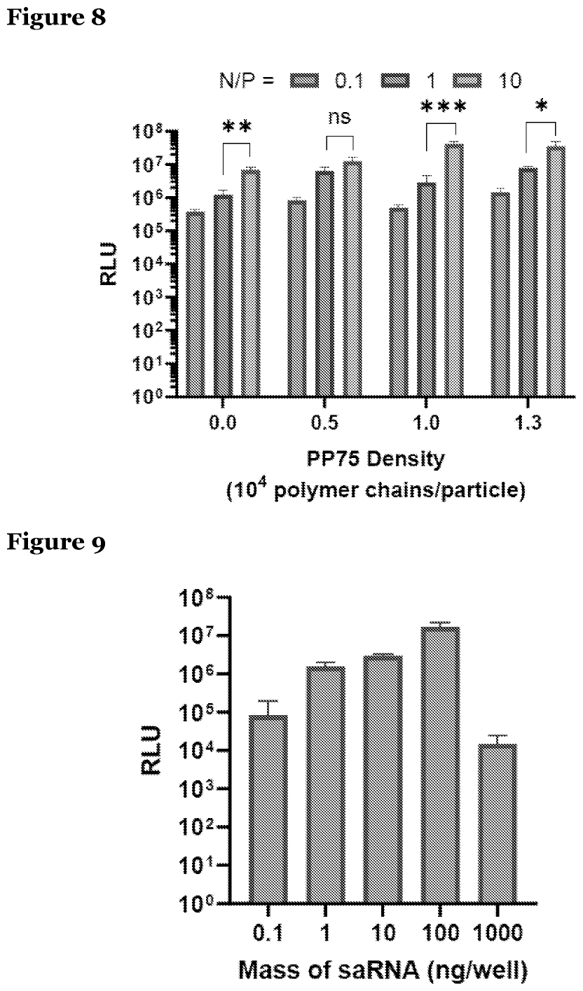

FIG. 8 shows HEK-293 cell transfection after 3 h of incubation with PF-LNPs (DOTAP/DOPE=1:1, 40 wt % cholesterol and various PP75 coating densities) at N/P ratio of 0.1, 1 and 10, respectively, as determined by Firefly Luciferase (fLuc) assay. Luciferase expression was evaluated 24 hours after transfection, expressed as relative light units (RLU). Mean±S.D. (n=3). *p<0.05, **p<0.01, ***p<0.001;

FIG. 9 shows Hela cell transfection after 3 h of incubation with PF-LNPs (DOTAP/DOPE=1:1, 40 wt % cholesterol and 1.3×10⁴ PP75 polymer chains/particle) at various concentrations of saRNA, as determined by Firefly Luciferase (fLuc) assay. Luciferase expression was evaluated 24 hours after transfection, expressed as relative light units (RLU). N/P ratio=1. Mean±S.D. (n=3);

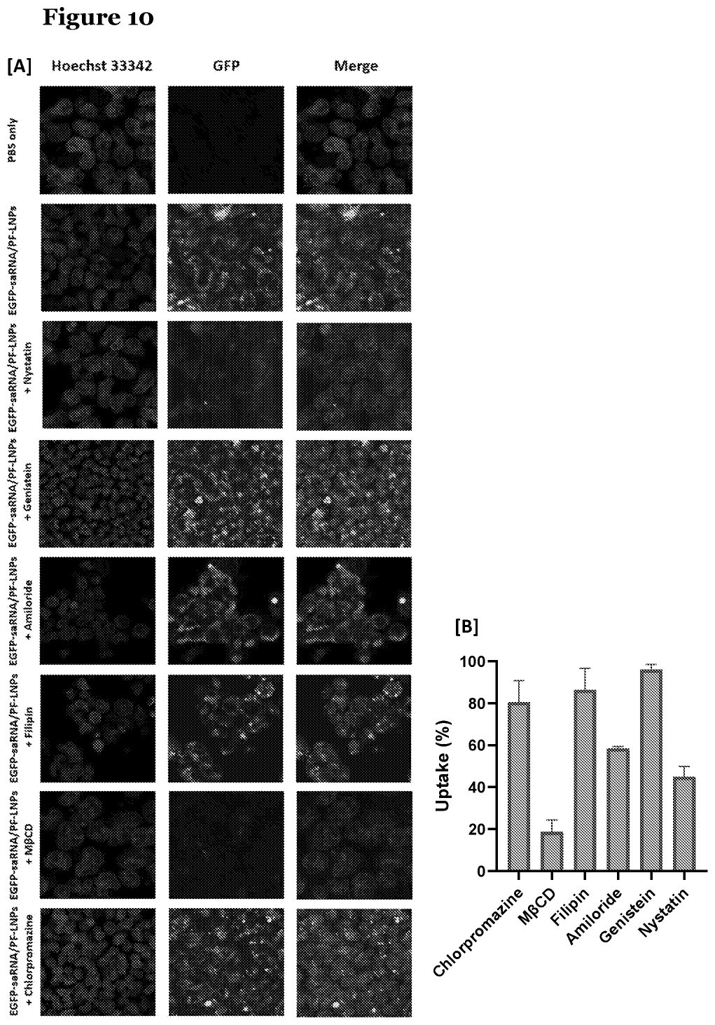

FIG. 10 provides laser scanning confocal microscopy images of HEK-293 cells when incubated with saRNA encoding enhanced green fluorescent protein (EGFP-saRNA) encapsulated PF-LNPs coated with PP75 at 1.3×10⁴ polymer chains/particle (N/P ratio=1) and various pathway inhibitors [A]. Quantitative representation [B];

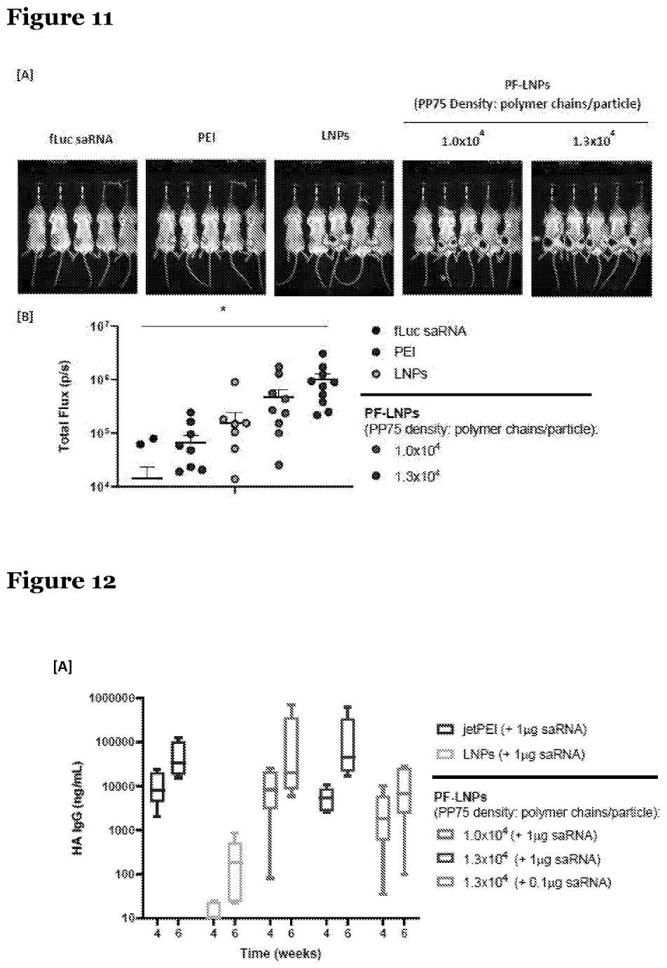

FIG. 11 provides in vivo visualization of fLuc bioluminescence in Balb/C female mice on day 7 after intramuscular injection with 5 μg of fLuc saRNA per leg. Images show (left to right): saRNA only, saRNA/PEI complexes, saRNA-encapsulated LNPs without PP75 functionalization (DOTAP/DOPE=1:1, 40 wt % cholesterol), and saRNA-encapsulated PF-LNPs consisting of DOTAP/DOPE (1:1), 40 wt % cholesterol and the PP75 density at 1.0×10⁴ and 1.3×10⁴ polymer chains/particle, respectively [A]. Quantification of luciferase expression with a line at the mean±standard deviation for n=5 mice (n=10 legs) per group [B]. N/P ratio=1. *p<0.05;

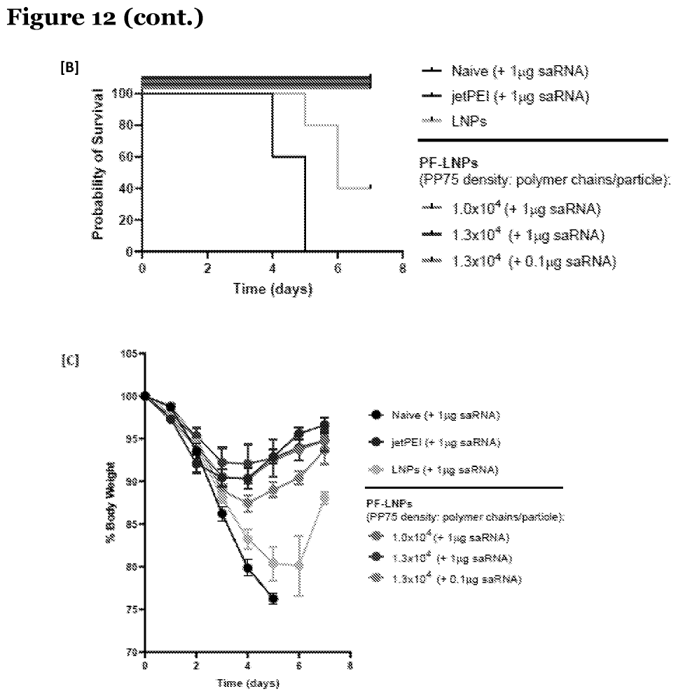

FIG. 12 shows in vivo evaluation of immunogenicity of the PF-LNPs encapsulated with hemagglutinin influenza virus encoded saRNA (HA-saRNA) in Balb/C female mice over 6 weeks. Antibody titers after immunization with HA-saRNA encapsulated in PF-LNPs, complexed with jetPEI and naïve, as determined by ELISA for n=5 mice at each time point [A], percentage survival of mice [B], and percentage body weight of mice throughout the duration of the study [C];

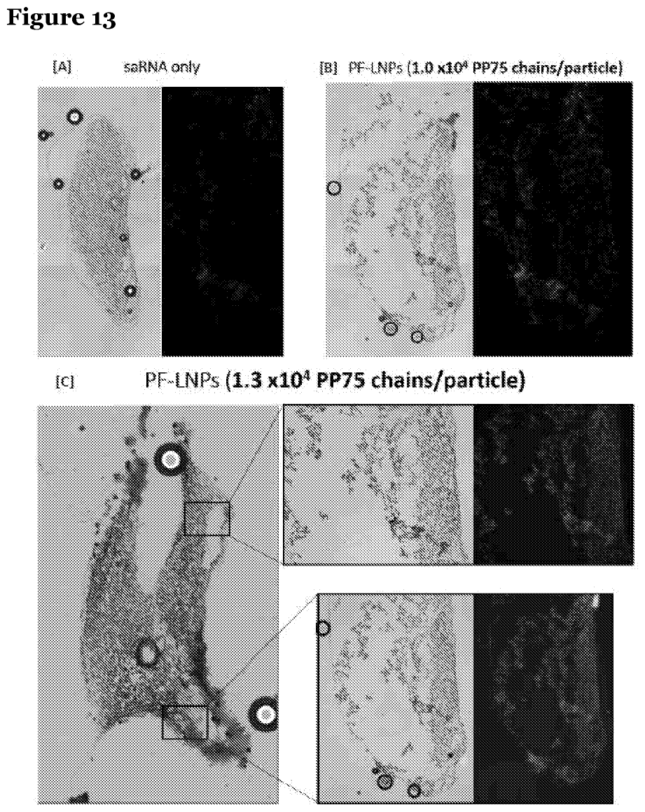

FIG. 13 shows lymphatic vessels of mouse lymph node 1 h after administering EGFP-encoding saRNA only [A], EGFP-saRNA encapsulated in PF-LNPs coated in 1.0×10⁴ polymer chains/particle Cy5-PP75 [B] and 1.3×10⁴ polymer chains/particle Cy5-PP75 [C]. Fluorescence images show EGFP-saRNA expression;

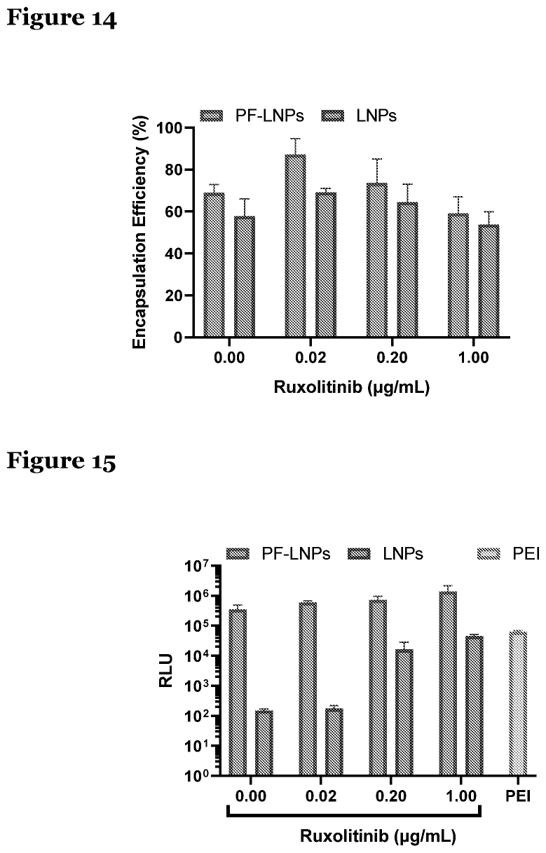

FIG. 14 shows the effect of Ruxolitinib concentration, co-delivered with LNPs without polymer functionalization (DOTAP/DOPE=1:1, 40 wt % cholesterol) and PF-LNPs (DOTAP/DOPE=1:1, 40 wt % cholesterol and 1.3×10⁴ PP75 polymer chains/particle), on saRNA encapsulation efficiency, as determined by RiboGreen™ Assay. (N/P ratio=1, and PEI as a control). Mean±SD (n=3);

FIG. 15 shows the effect of Ruxolitinib concentration, co-delivered with LNPs without polymer functionalization (DOTAP/DOPE=1:1, 40 wt % cholesterol) and PF-LNPs (DOTAP/DOPE=1:1, 40 wt % cholesterol and 1.3×10⁴ PP75 polymer chains/particle) on MRC-5 cell transfection (N/P ratio=1, and PEI as a control). Mean±SD (n=3);

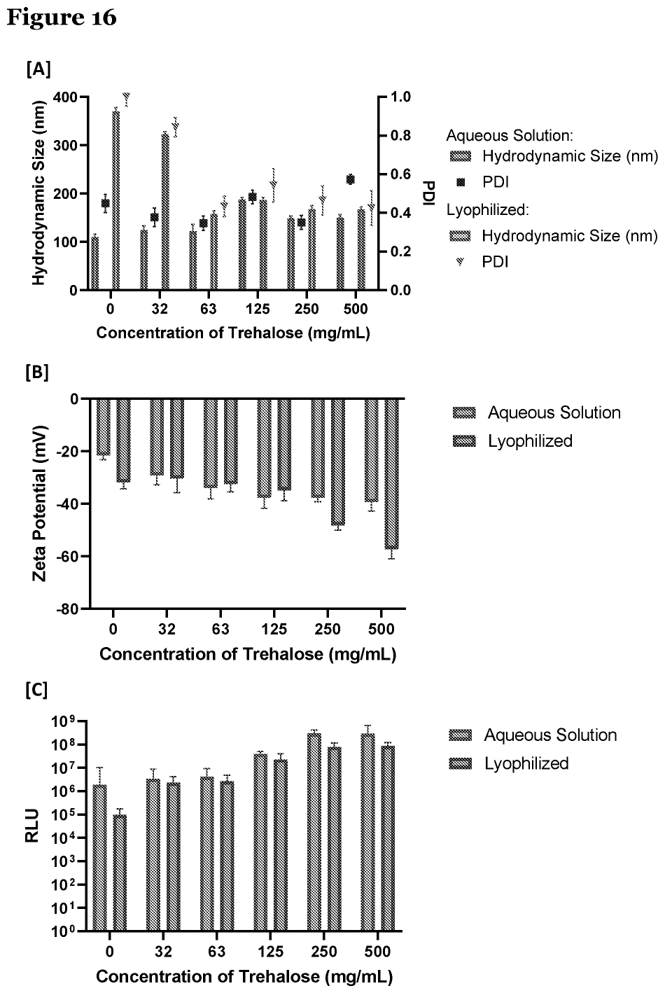

FIG. 16 provides evaluation of the formulations, in aqueous solution and lyophilized, of saRNA-encapsulated PF-LNPs (DOTAP/DOPE=1:1, 40 wt % cholesterol and 1.3×10⁴ PP75 polymer chains/particle), with co-existence of trehalose both inside and outside PF-LNPs. Hydrodynamic size and polydispersity index (PDI) of the formulations [A], zeta potential [B] and HEK-293 cell transfection of saRNA as determined by Firefly Luciferase (fLuc) assay [C]. (N/P ratio=1). Mean±SD (n=3);

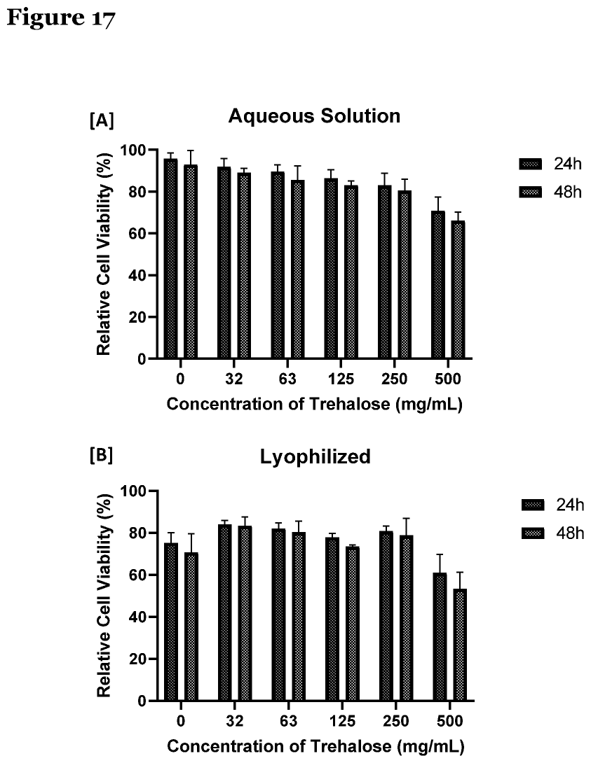

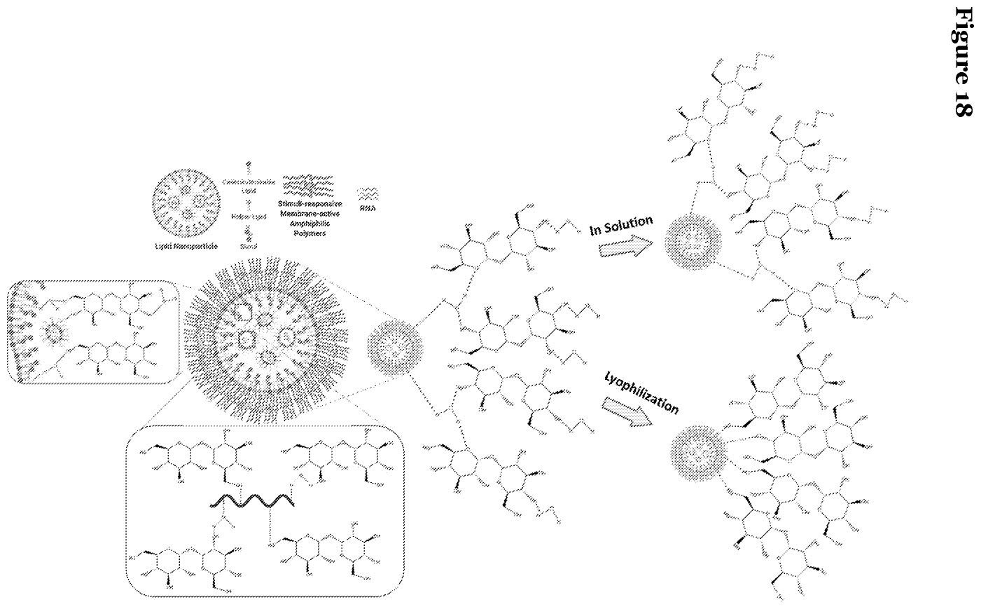

FIG. 17 shows relative HEK-293 cell viability after 24 and 48 h of incubation, respectively, with the formulations, in aqueous solution [A] and lyophilized [B], of saRNA-encapsulated PF-LNPs (DOTAP/DOPE=1:1, 40 wt % cholesterol and 1.3×10⁴ PP75 polymer chains/particle), with co-existence of trehalose both inside and outside PF-LNPs, as determined by Alamar Blue assay. N/P ratio=1;

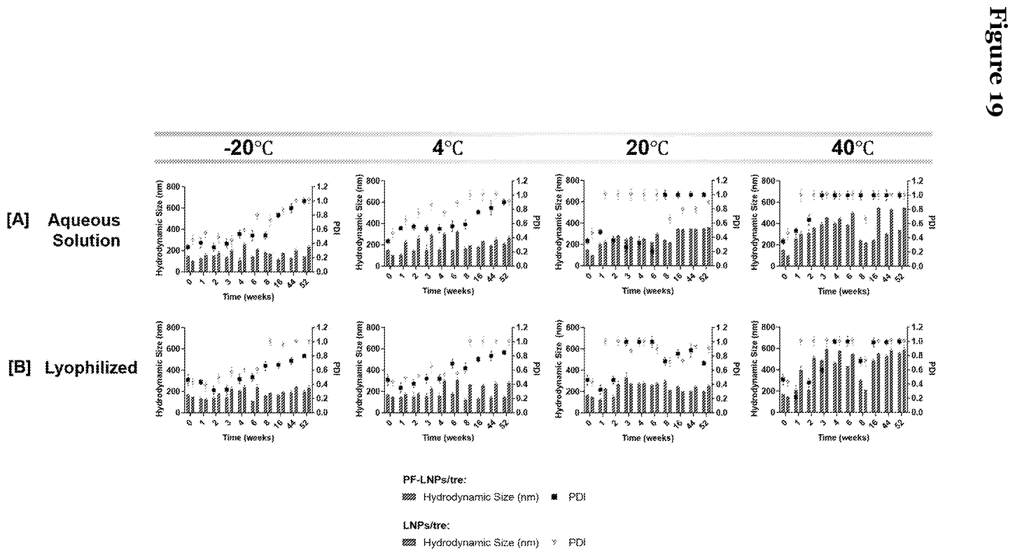

FIG. 18 is a schematic showing interactions of trehalose with RNA-encapsulated PF-LNPs in solution and after lyophilization;

FIG. 19 provides evaluation of storage stability of the formulations, in aqueous solution and lyophilized, of saRNA-encapsulated PF-LNPs coated with 1.3×10⁴ PP75 polymer chains/particle, with co-existence of trehalose both inside and outside PF-LNPs. Hydrodynamic size and polydispersity index (PDI) of the formulations over 52 weeks of storage at −20° C., 4° C., 20° C. and 40° C., respectively. (N/P ratio=1). Mean t SD (n=3);

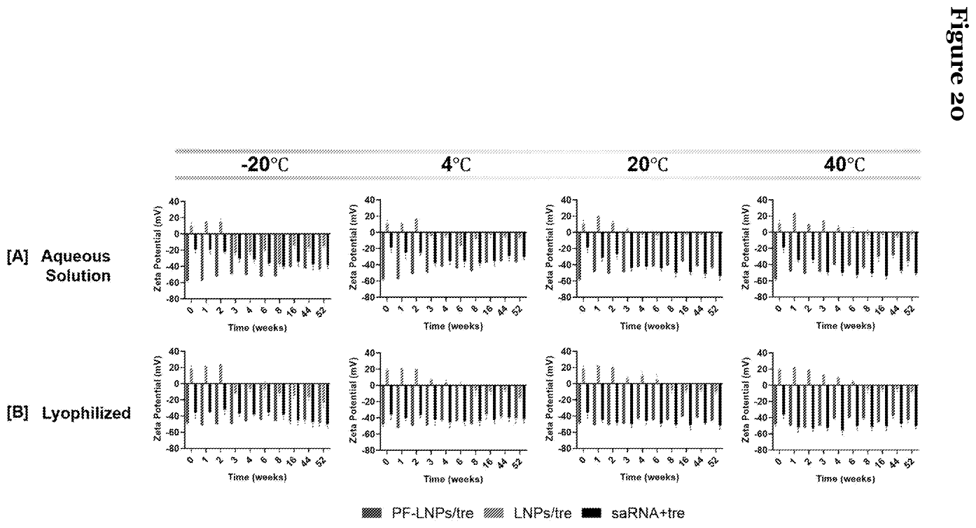

FIG. 20 provides evaluation of storage stability of the formulations, in aqueous solution and lyophilized, of saRNA-encapsulated PF-LNPs coated with 1.3×10⁴ PP75 polymer chains/particle, with co-existence of trehalose both inside and outside PF-LNPs. Zeta potential of the formulations over 52 weeks of storage at −20° C., 4° C., 20° C. and 40° C., respectively. (N/P ratio=1). Mean±SD (n=3);

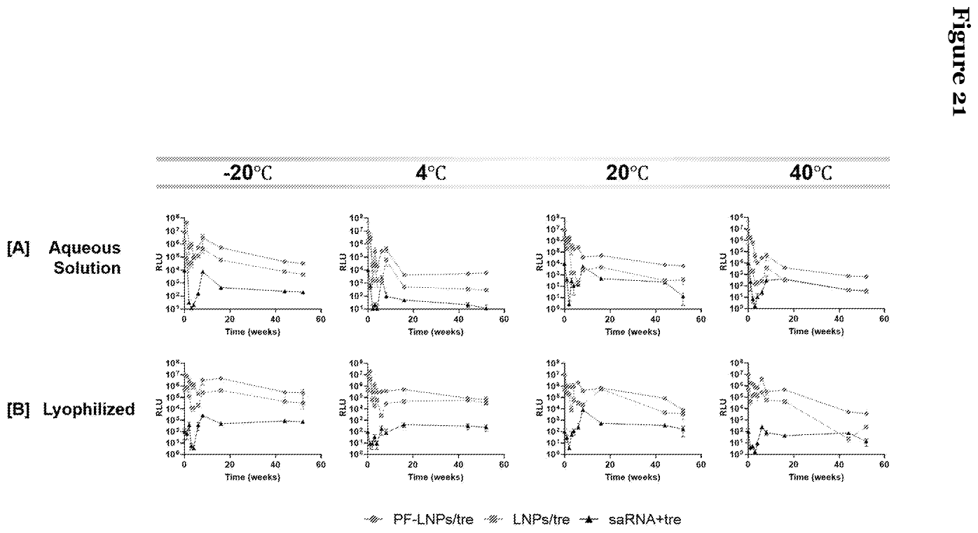

FIG. 21 provides evaluation of storage stability of the formulations, in aqueous solution and lyophilized, of saRNA-encapsulated PF-LNPs coated with 1.3×10⁴ PP75 polymer chains/particle, with co-existence of trehalose both inside and outside PF-LNPs. HEK-293 cell transfection of saRNA over 52 weeks of storage at −20° C., 4° C., 20° C. and 40° C., respectively, as determined by Firefly Luciferase (fLuc) assay. (N/P ratio=1). Mean±SD (n=3);

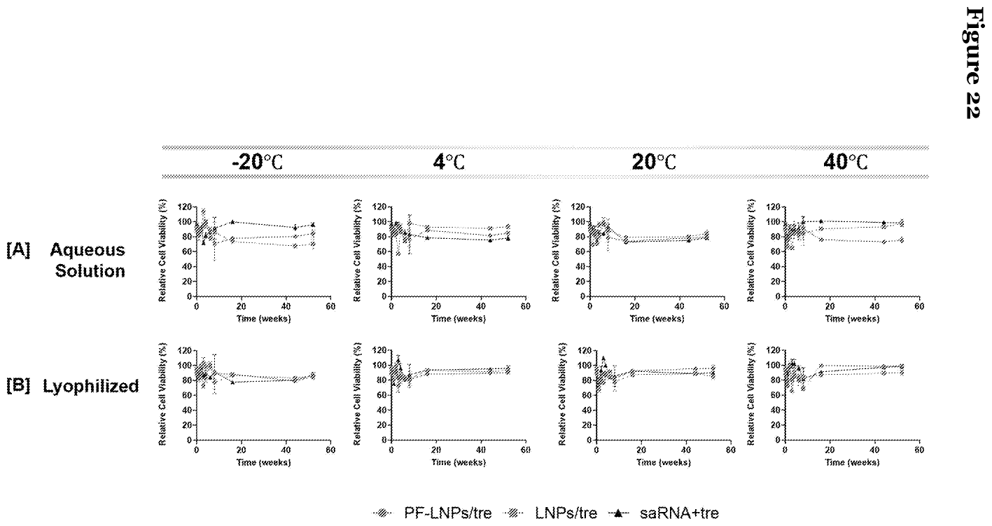

FIG. 22 provides evaluation of storage stability of the formulations, in aqueous solution and lyophilized, of saRNA-encapsulated PF-LNPs coated with 1.3×10⁴ PP75 polymer chains/particle, with co-existence of trehalose both inside and outside PF-LNPs. Relative cell viability of HEK-293 cells after 48 h of treatment with the formulations over 52 weeks of storage at −20° C., 4° C., 20° C. and 40° C., respectively, as measured using Alamar Blue assay. (N/P ratio=1). Mean±SD (n=3);

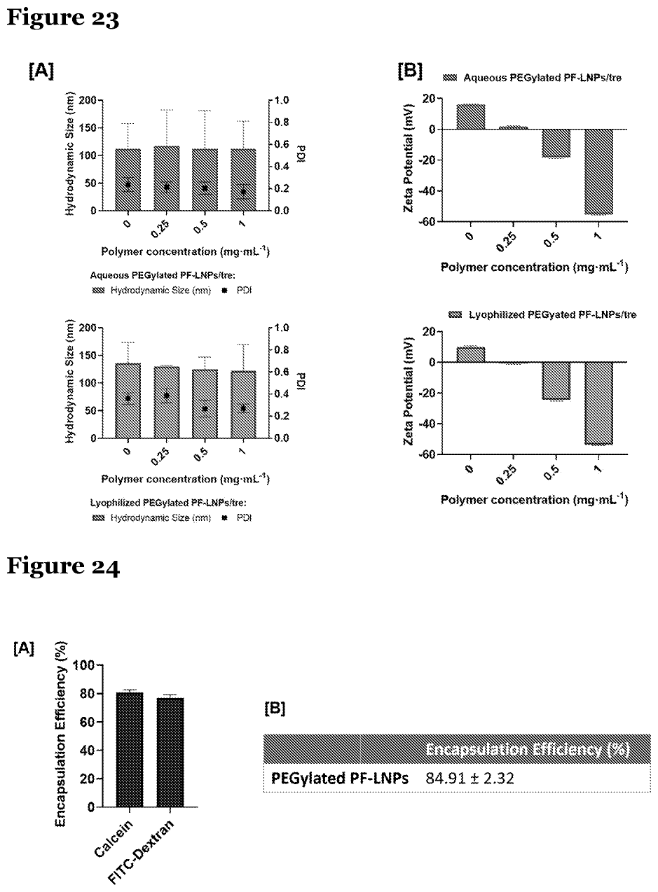

FIG. 23 shows the physiochemical characterization of PEGylated PF-LNPs (DOTAP/DOPE/DMG-PEG2000/Cholesterol and varying PP75 polymer coating densities) at pH 7.4: mean hydrodynamic diameters and PDI values [A] and zeta potentials [B] of formulations in aqueous solution and lyophilized form. Mean±SD (n=3);

FIG. 24 shows the Calcein and FITC-Dextran (Mw=150 kDa) encapsulation efficiency in PEGylated LNPs (DOTAP/DOPE/DMG-PEG2000/Cholesterol) [A] and fLuc-saRNA encapsulation efficiency as determined by the RiboGreen assay [B];

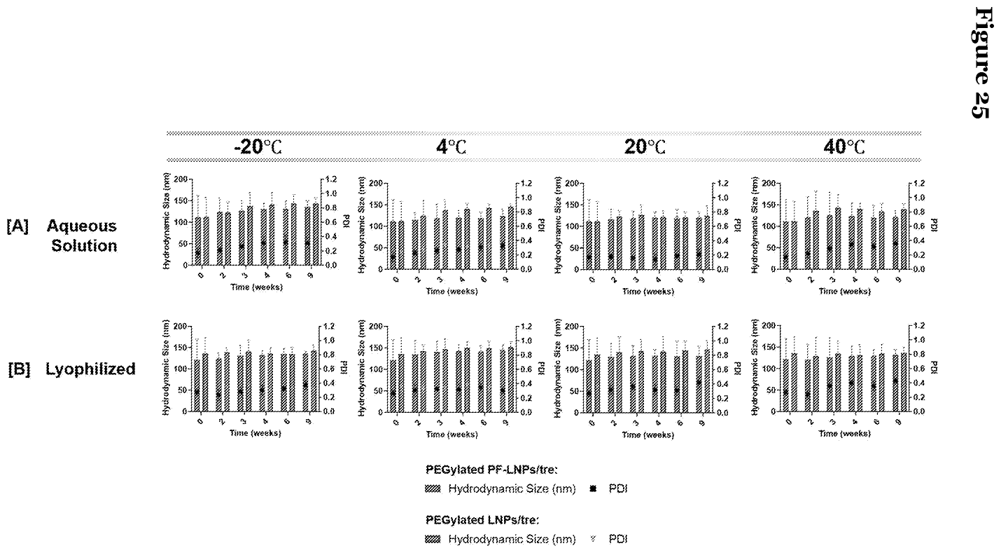

FIG. 25 provides evaluation of storage stability of the formulations, in aqueous solution and lyophilized, of saRNA-encapsulated PEGylated PF-LNPs (DOTAP/DOPE/DMG-PEG2000/Cholesterol with 1 mg/mL PP75), with co-existence of trehalose both inside and outside the PEGylated PF-LNPs. Hydrodynamic size and polydispersity index (PDI) of the formulations over 9 weeks of storage at −20° C., 4° C., 20° C. and 40° C., respectively. (N/P ratio=1). Mean±SD (n=3);

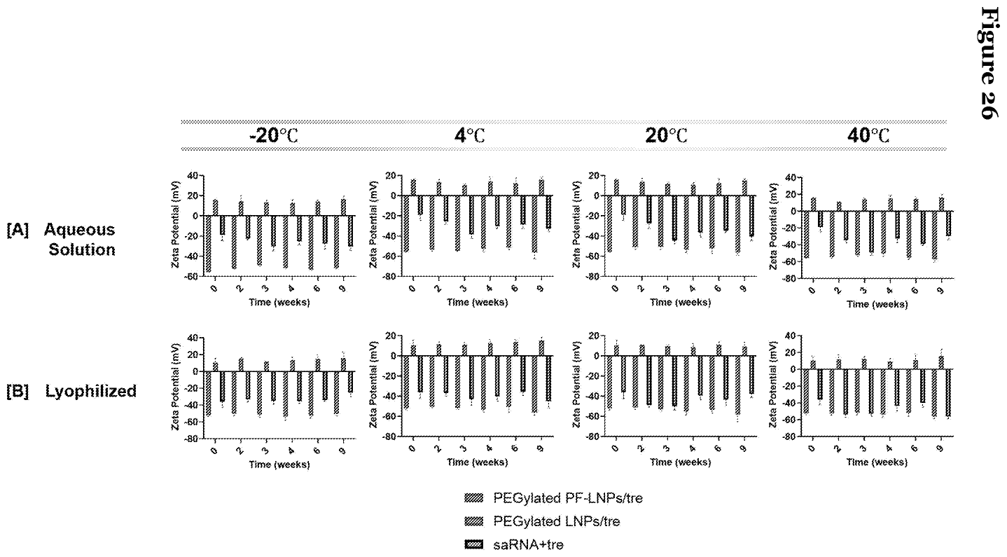

FIG. 26 provides evaluation of storage stability of the formulations, in aqueous solution and lyophilized, of saRNA-encapsulated PEGylated PF-LNPs (DOTAP/DOPE/DMG-PEG2000/Cholesterol with 1 mg/mL PP75), with co-existence of trehalose both inside and outside the PEGylated PF-LNPs. Zeta potential of the formulations over 9 weeks of storage at −20° C., 4° C., 20° C. and 40° C., respectively. (N/P ratio=1). Mean±SD (n=3);

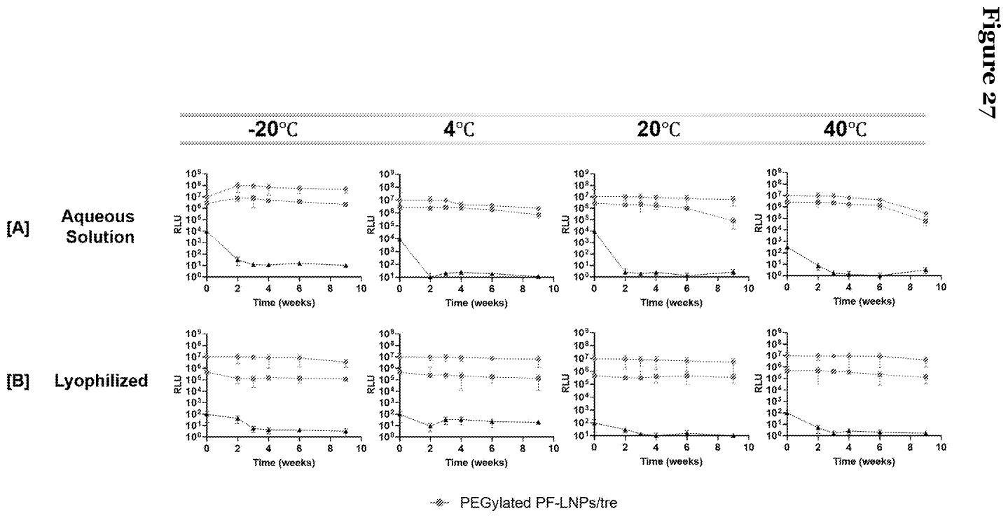

FIG. 27 provides evaluation of storage stability of the formulations, in aqueous solution and lyophilized, of saRNA-encapsulated PEGylated PF-LNPs (DOTAP/DOPE/DMG-PEG2000/Cholesterol with 1 mg/mL PP75), with co-existence of trehalose both inside and outside the PEGylated PF-LNPs. HEK-293 cell transfection of saRNA over 9 weeks of storage at −20° C., 4° C., 20° C. and 40° C., respectively, as determined by Firefly Luciferase (fLuc) assay. (N/P ratio=1). Mean±SD (n=3);

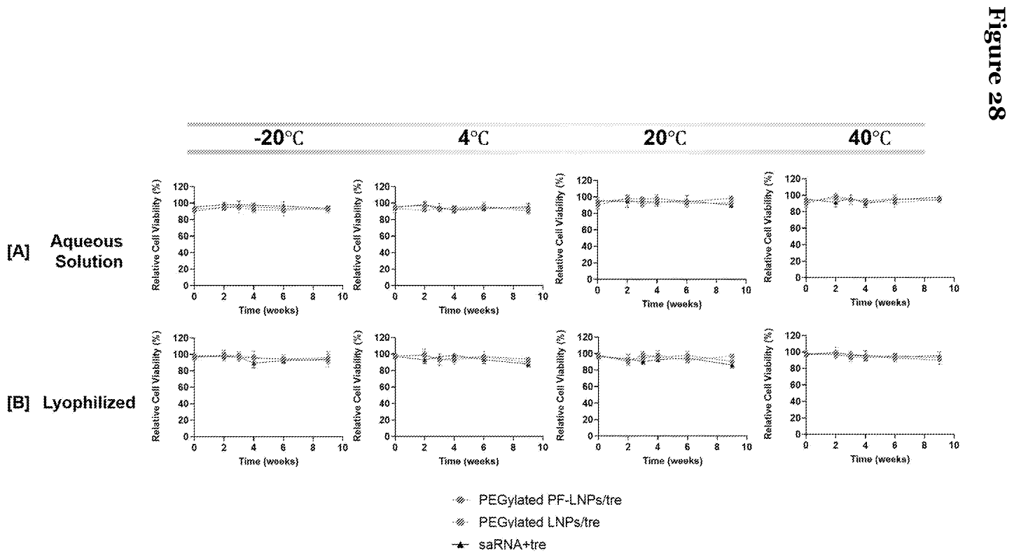

FIG. 28 provides evaluation of storage stability of the formulations, in aqueous solution and lyophilized, of saRNA-encapsulated PEGylated PF-LNPs (DOTAP/DOPE/DMG-PEG2000/Cholesterol with 1 mg/mL PP75), with co-existence of trehalose both inside and outside the PEGylated PF-LNPs. Relative cell viability of HEK-293 cells after 48 h of treatment with the formulations over 9 weeks of storage at −20° C., 4° C., 20° C. and 40° C., respectively, as measured using Alamar Blue assay. (N/P ratio=1). Mean±SD (n=3);

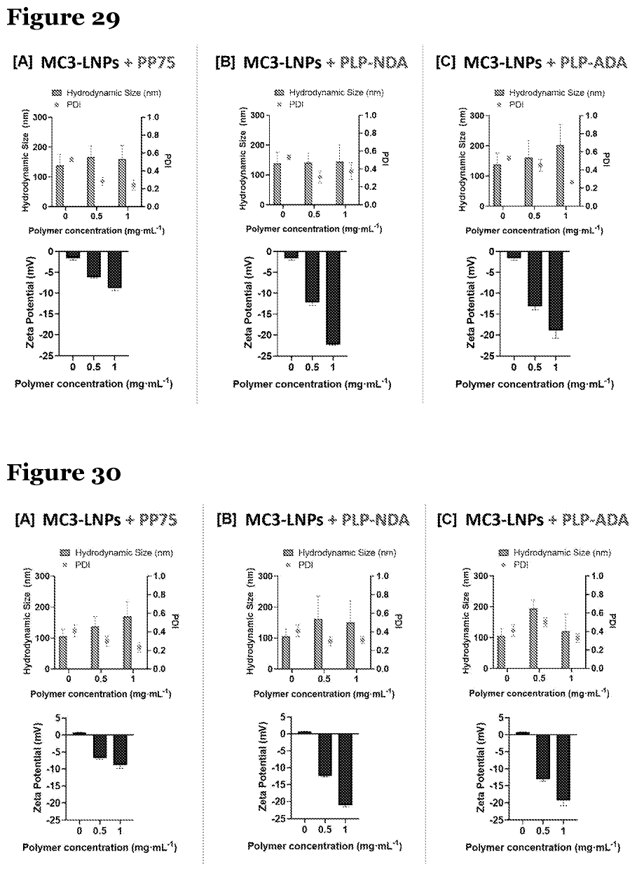

FIG. 29 shows the physiochemical characterization (mean hydrodynamic diameters, PDI values and zeta potentials) of MC3 PF-LNPs (MC3/DSPC/Chol/DMG-PEG2000) with varying concentrations of PP75 [A], PLP-NDA [B] and PLP-ADA [C] polymers, at pH 7.4, without saRNA: Mean±SD (n=3);

FIG. 30 shows the physiochemical characterization (mean hydrodynamic diameters, PDI values and zeta potentials) of MC3 PF-LNPs (MC3/DSPC/Chol/DMG-PEG2000) with varying concentrations of PP75 [A], PLP-NDA [B] and PLP-ADA [C] polymers, at pH 7.4, with saRNA: Mean±SD (n=3);

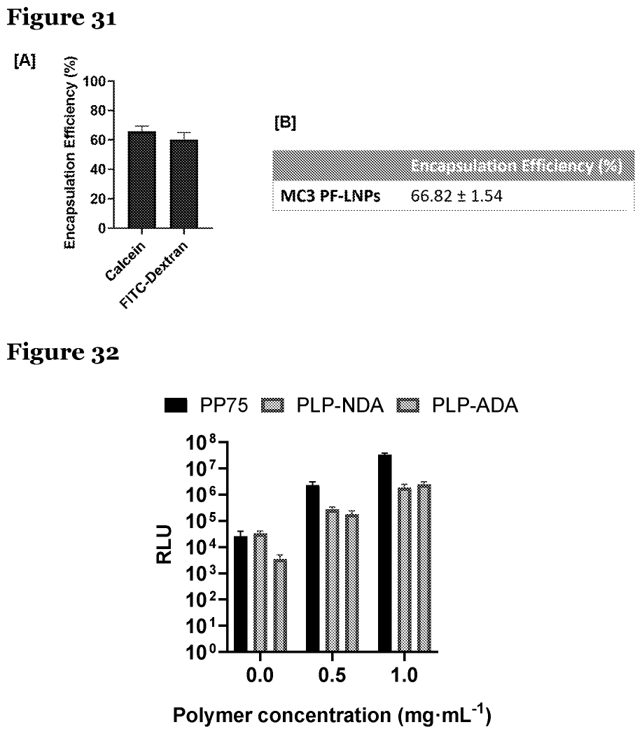

FIG. 31 shows the Calcein and FITC-Dextran (Mw=150 kDa) encapsulation efficiency in MC3 LNPs (MC3/DSPC/Chol/DMG-PEG2000) [A] and fLuc-saRNA encapsulation efficiency as determined by the RiboGreen assay [B];

FIG. 32 shows HEK-293 cell transfection of saRNA encapsulated in MC3 PF-LNPs (MC3/DSPC/Chol/DMG-PEG2000) with varying concentrations of PP75, PLP-NDA and PLP-ADA polymers, as determined by Firefly Luciferase (fLuc) assay. (N/P ratio=1). Mean±SD (n=3);

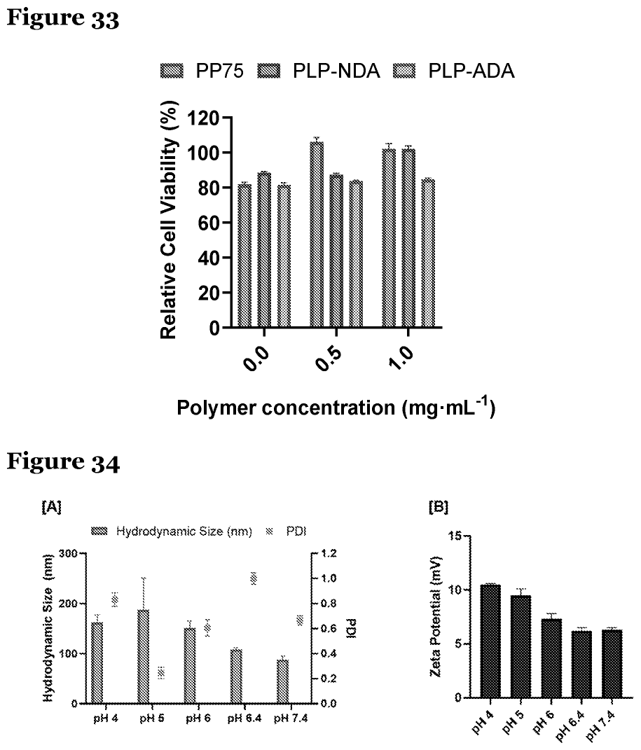

FIG. 33 shows relative cell viability of HEK-293 cells after 48 h of treatment with saRNA encapsulated in MC3 PF-LNPs (MC3/DSPC/Chol/DMG-PEG2000) with varying concentrations of PP75, PLP-NDA and PLP-ADA polymers, as measured using Alamar Blue assay. (N/P ratio=1). Mean±SD (n=3);

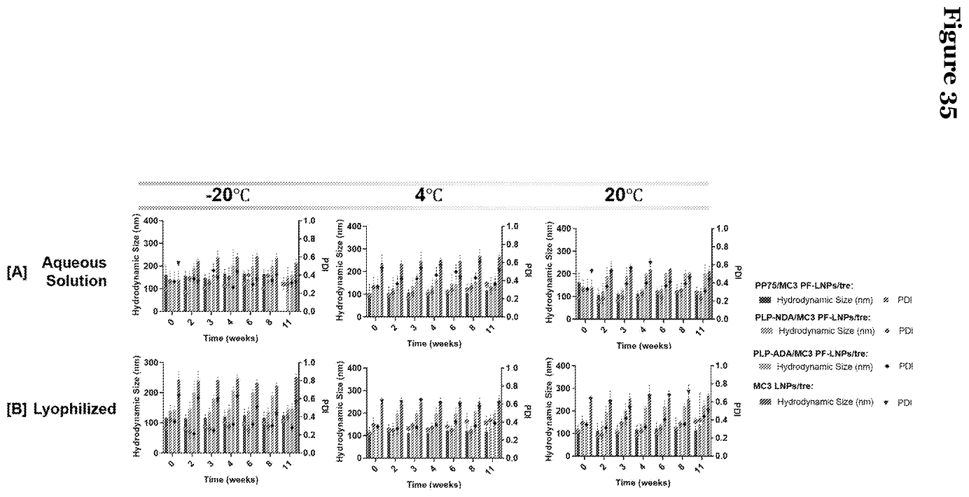

FIG. 34 shows the physiochemical characterization (mean hydrodynamic diameters and PDI values [A] and zeta potentials [B]) of MC3 LNPs (MC3/DSPC/Chol/DMG-PEG2000) at various pH values, without saRNA: Mean t SD (n=3);

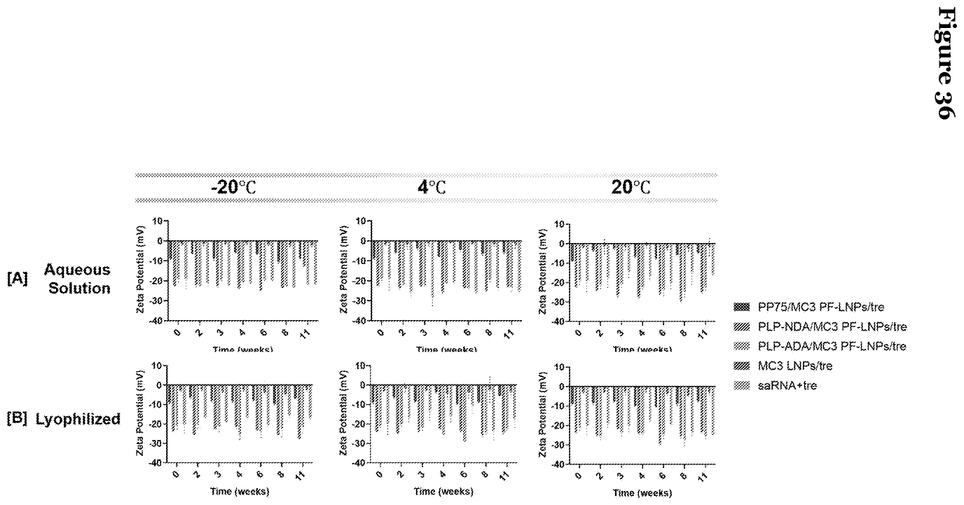

FIG. 35 provides evaluation of storage stability of the formulations, in aqueous solution and lyophilized, of saRNA-encapsulated MC3 PF-LNPs (MC3/DSPC/Chol/DMG-PEG2000) with 1 mg/mL PP75, PLP-NDA or PLP-ADA), with co-existence of trehalose both inside and outside the MC3 PF-LNPs.

Hydrodynamic size and polydispersity index (PDI) of the formulations over 11 weeks of storage at −20° C., 4° C. and 20° C., respectively. (N/P ratio=1). Mean±SD (n=3);

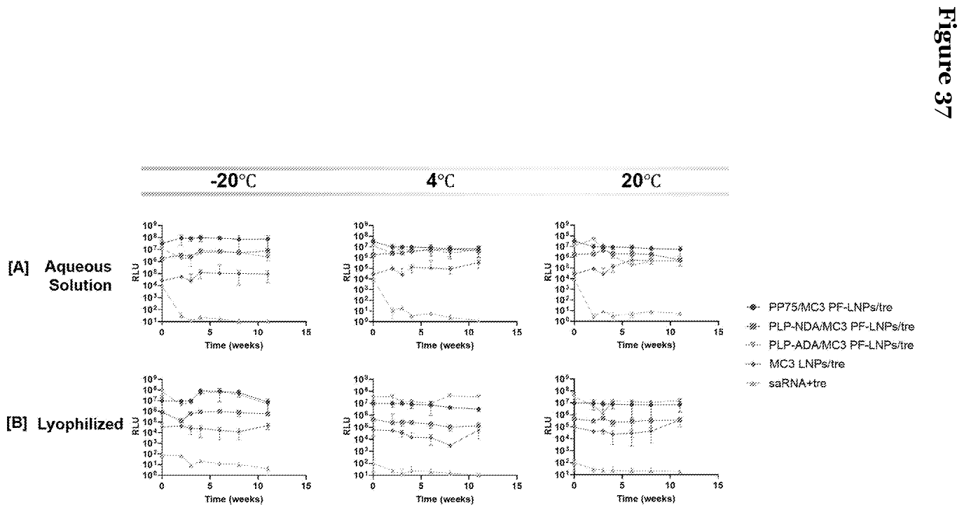

FIG. 36 provides evaluation of storage stability of the formulations, in aqueous solution and lyophilized, of saRNA-encapsulated MC3 PF-LNPs (MC3/DSPC/Chol/DMG-PEG2000) with 1 mg/mL PP75, PLP-NDA or PLP-ADA), with co-existence of trehalose both inside and outside the MC3 PF-LNPs. Zeta potential of the formulations over 11 weeks of storage at −20° C., 4° C., and 20° C., respectively. (N/P ratio=1). Mean±SD (n=3);

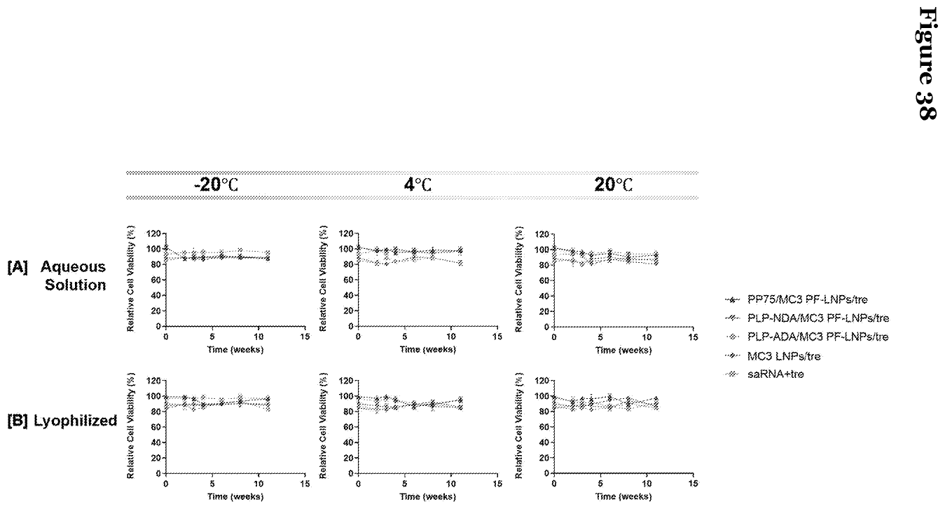

FIG. 37 provides evaluation of storage stability of the formulations, in aqueous solution and lyophilized, of saRNA-encapsulated MC3 PF-LNPs (MC3/DSPC/Chol/DMG-PEG2000) with 1 mg/mL PP75, PLP-NDA or PLP-ADA), with co-existence of trehalose both inside and outside the MC3 PF-LNPs. HEK-293 cell transfection of saRNA over 11 weeks of storage at −20° C., 4° C., and 20° C., respectively, as determined by Firefly Luciferase (fLuc) assay. (N/P ratio=1). Mean±SD (n=3);

FIG. 38 provides evaluation of storage stability of the formulations, in aqueous solution and lyophilized, of saRNA-encapsulated MC3 PF-LNPs (MC3/DSPC/Chol/DMG-PEG2000) with 1 mg/mL PP75, PLP-NDA or PLP-ADA), with co-existence of trehalose both inside and outside the MC3 PF-LNPs. Relative cell viability of HEK-293 cells after 48 h of treatment with the formulations over 11 weeks of storage at −20° C., 4° C. and 20° C., respectively, as measured using Alamar Blue assay. (N/P ratio=1). Mean±SD (n=3);

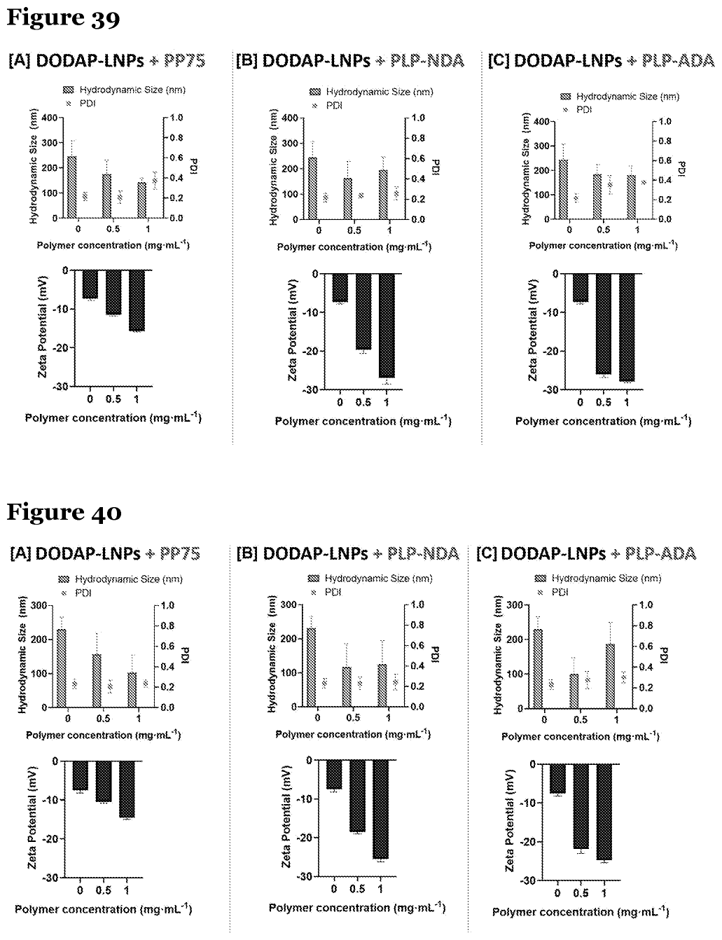

FIG. 39 shows the physiochemical characterization (mean hydrodynamic diameters, PDI values and zeta potentials) of DODAP PF-LNPs (DODAP/DOPE/Chol/DMG-PEG2000) with varying concentrations of PP75 [A], PLP-NDA [B] and PLP-ADA [C] polymers, at pH 7.4, without saRNA: Mean±SD (n=3);

FIG. 40 shows the physiochemical characterization (mean hydrodynamic diameters, PDI values and zeta potentials) of DODAP PF-LNPs (DODAP/DOPE/Chol/DMG-PEG2000) with varying concentrations of PP75 [A], PLP-NDA [B] and PLP-ADA [C] polymers, at pH 7.4, with saRNA: Mean±SD (n=3);

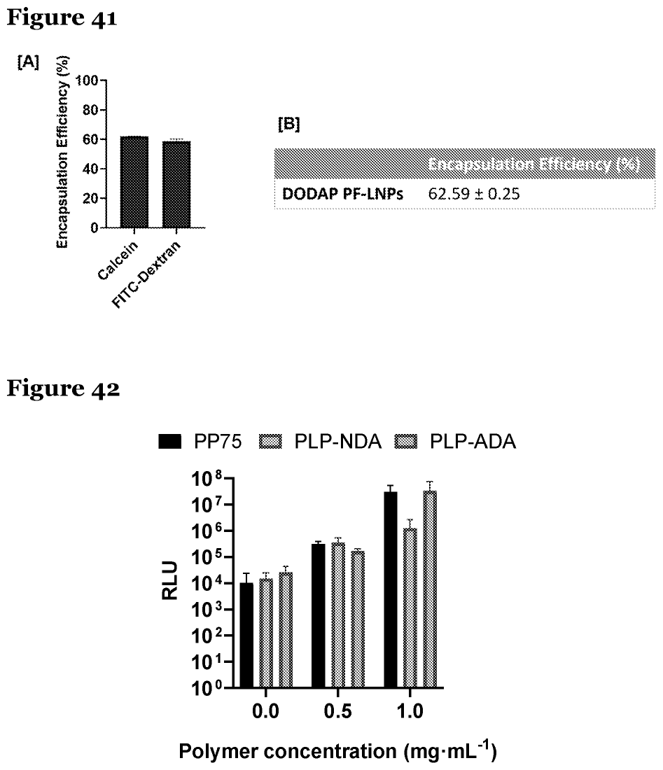

FIG. 41 shows the Calcein and FITC-Dextran (Mw=150 kDa) encapsulation efficiency in DODAP LNPs (DODAP/DOPE/Chol/DMG-PEG2000) [A] and fLuc-saRNA encapsulation efficiency as determined by the RiboGreen assay [B].

FIG. 42 shows HEK-293 cell transfection of saRNA encapsulated in DODAP PF-LNPs (DODAP/DOPE/Chol/DMG-PEG2000) with varying concentrations of PP75, PLP-NDA and PLP-ADA polymers, as determined by Firefly Luciferase (fLuc) assay. (N/P ratio=1). Mean±SD (n=3);

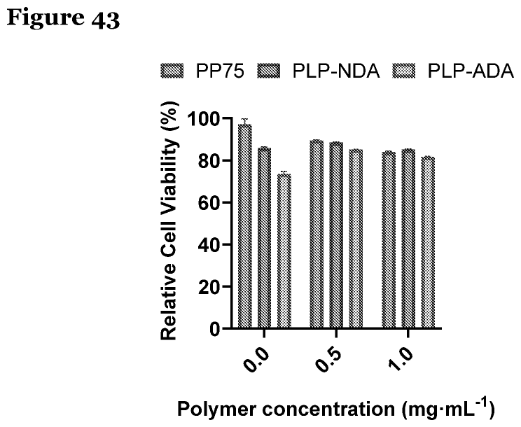

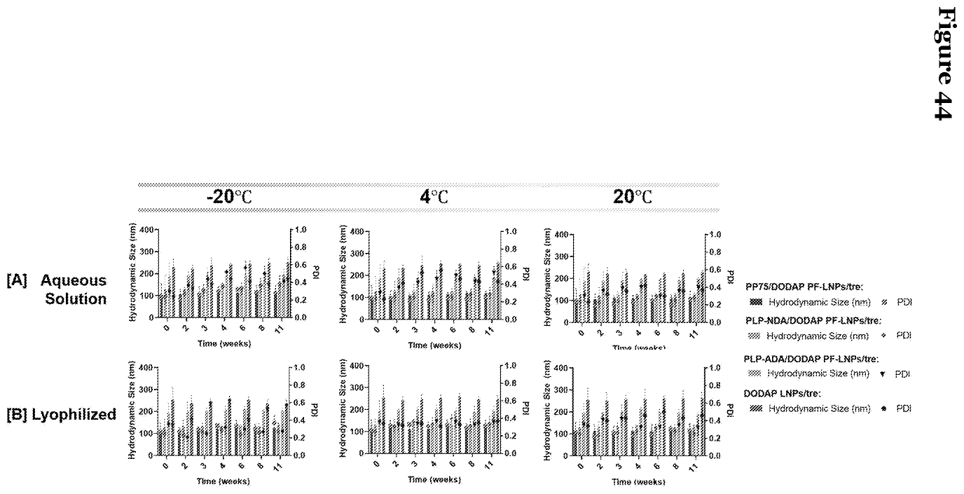

FIG. 43 shows relative cell viability of HEK-293 cells after 48 h of treatment with saRNA encapsulated in DODAP PF-LNPs (DODAP/DOPE/Chol/DMG-PEG2000) with varying concentrations of PP75, PLP-NDA and PLP-ADA polymers, as measured using Alamar Blue assay. (N/P ratio=1). Mean±SD (n=3);

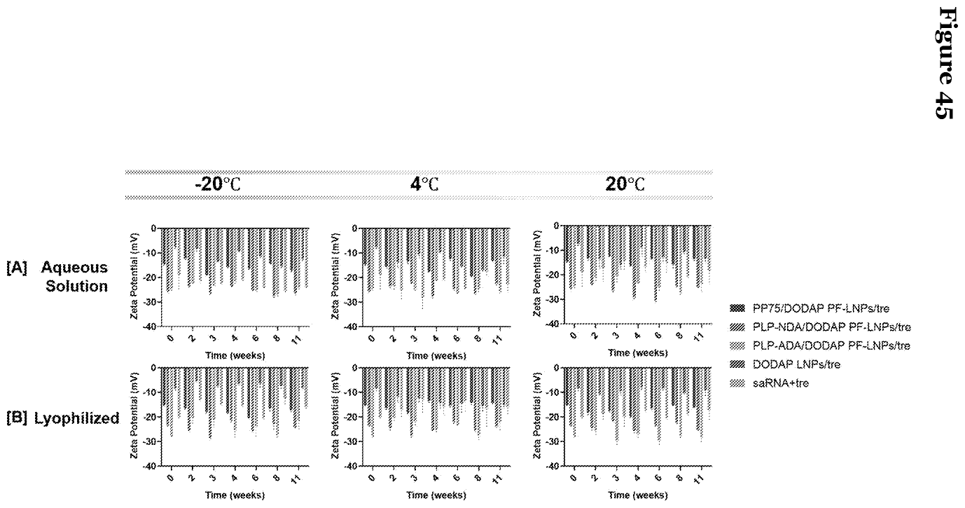

FIG. 44 provides evaluation of storage stability of the formulations, in aqueous solution and lyophilized, of saRNA-encapsulated DODAP PF-LNPs (DODAP/DOPE/Chol/DMG-PEG2000) with 1 mg/mL PP75, PLP-NDA or PLP-ADA), with co-existence of trehalose both inside and outside the DODAP PF-LNPs. Hydrodynamic size and polydispersity index (PDI) of the formulations over 11 weeks of storage at −20° C., 4° C. and 20° C., respectively. (N/P ratio=1). Mean±SD (n=3);

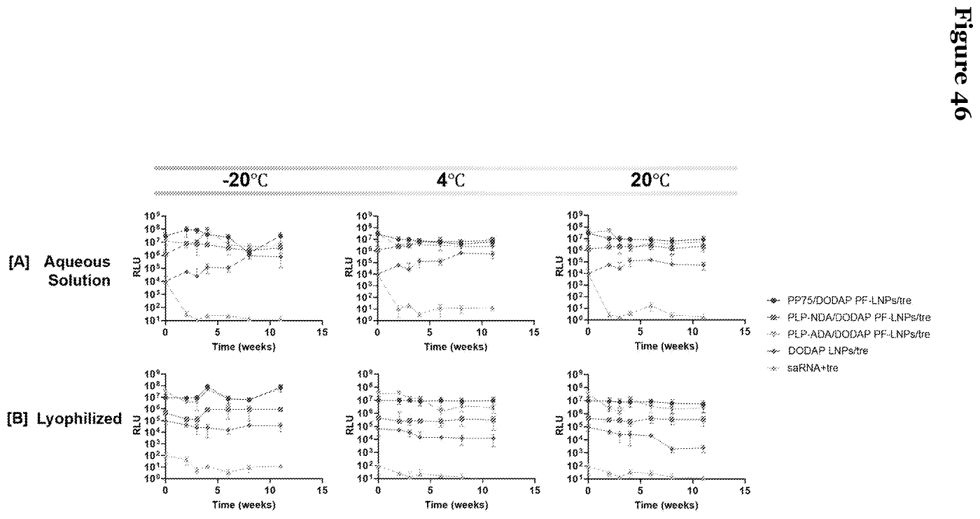

FIG. 45 provides evaluation of storage stability of the formulations, in aqueous solution and lyophilized, of saRNA-encapsulated DODAP PF-LNPs (DODAP/DOPE/Chol/DMG-PEG2000) with 1 mg/mL, with co-existence of trehalose both inside and outside the DODAP PF-LNPs. Zeta potential of the formulations over 11 weeks of storage at −20° C., 4° C., and 20° C., respectively. (N/P ratio=1). Mean±SD (n=3);

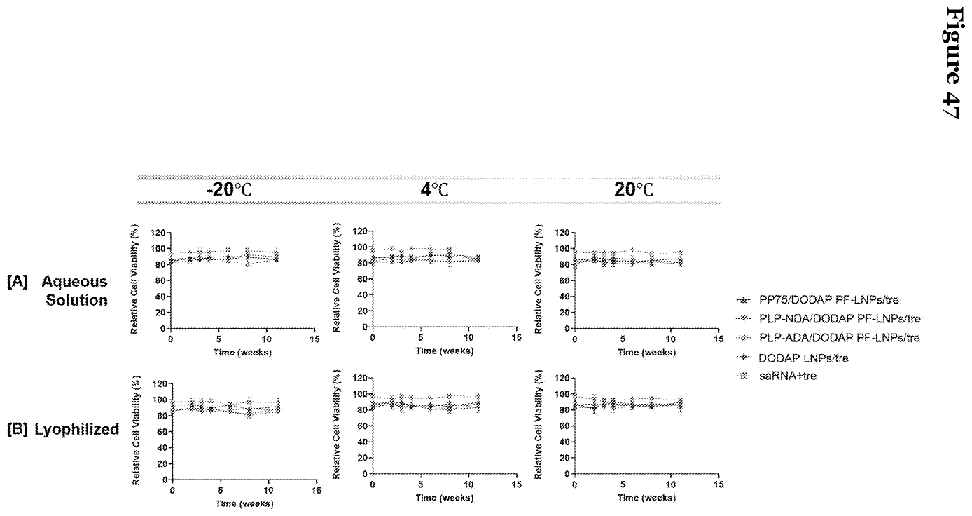

FIG. 46 provides evaluation of storage stability of the formulations, in aqueous solution and lyophilized, of saRNA-encapsulated DODAP PF-LNPs (DODAP/DOPE/Chol/DMG-PEG2000) with 1 mg/mL PP75, PLP-NDA or PLP-ADA), with co-existence of trehalose both inside and outside the DODAP PF-LNPs. HEK-293 cell transfection of saRNA over 11 weeks of storage at −20° C., 4° C., and 20° C., respectively, as determined by Firefly Luciferase (fLuc) assay. (N/P ratio=1). Mean t SD (n=3); and

FIG. 47 provides evaluation of storage stability of the formulations, in aqueous solution and lyophilized, of saRNA-encapsulated DODAP PF-LNPs (DODAP/DOPE/Chol/DMG-PEG2000) with 1 mg/mL PP75, PLP-NDA or PLP-ADA), with co-existence of trehalose both inside and outside the DODAP PF-LNPs. Relative cell viability of HEK-293 cells after 48 h of treatment with the formulations over 11 weeks of storage at −20° C., 4° C. and 20° C., respectively, as measured using Alamar Blue assay. (N/P ratio=1). Mean±SD (n=3).

### EXAMPLE 1—PREPARATION & CHARACTERIZATION OF PF-LNPS, HIGH BIOCOMPATIBILITY & LOW CYTOTOXICITY, AND HIGH IN VITRO TRANSFECTION EFFICIENCY VIA EFFICIENT INTRACELLULAR RNA DELIVERY

The inventors synthesised polymer functionalized lipid nanoparticles (PF-LNPs) as explained in the Materials and Methods section below. The PF-LNPs were coated with a biocompatible, pH-responsive, endosomolytic pseudopeptidic polymer, called PP75, which mimics the anionic fusogenic peptides in the hemagglutinin spikes of influenza virus.

PP75 is a pseudopeptide prepared by grafting hydrophobic amino acid L-phenylalanine to carboxylic acids pendant to the backbone of a metabolite-derived linear polyamide, poly(L-lysine iso-phthalamide) (PLP), at 75% stoichiometric degree of substitution. The biomimetic anionic amphiphilic polymer contains hydrophobic pendant groups and ionizable carboxylic acid groups found in the fusogenic peptides of influenza virus. The inventors envisaged that PP75 might work as a pseudopeptide on the surface of the particle to mimic the viral spikes and thereby deliver the PF-LNPs into the cell cytoplasm via endosomal escape, as shown in FIG. 1.

### Polymer Coating Efficiency

As shown in FIG. 2A, it was found that use of 1 mg/mL PP75 produced a PP75 polymer density of 1.3×10⁴ polymer chains/particle on the surface of the PF-LNPs (DOTAP/DOPE weight ratio=1:1, 40 wt % cholesterol) when encapsulated with saRNA. FIG. 2B shows that up to 1.0×10⁴ polymer chains/particles were completely coated on the surface of PF-LNPs. At 1.3×10⁴ polymer chains/particles, the PP75 coating efficiency was decreased to only 50.3±0.4%, suggesting that the particle surface was saturated with the PP75. It was decided that, for all in vivo studies, the focus would be on PF-LNPs with between 1.0×10⁴ and 1.3×10⁴ polymer chains/particle, to ensure only saturated particles were used for the purpose of maximizing the negative surface charge and endosomolytic capacity.

### Physiochemical Characterization of PF-LNPs

FIG. 3A shows the effect of polymer density on the hydrodynamic diameter of PF-LNPs. The average particle size was found to be similar at 110.0±5.6 nm for the PF-LNPs coated with PP75 at 0.5×10⁴, 1.0×10⁴ and 1.3×10⁴ polymer chains/particles, and the size of PF-LNPs was slightly larger than the LNPs without PP75 coating, which is as expected according to previous studies⁹. It has been demonstrated that particles with sizes of 200 nm or below can enter cells via clathrin-coated pits in the cell membrane¹⁰. FIG. 3B shows the concentration and size distribution of the particles. The concentration of the PF-LNPs with 40 wt % cholesterol was 2.9×10⁹±0.7×10⁹ particles/mL. The more concentrated the particles are, the higher the saRNA dose can be administered in a smaller volume. The particles had a narrow size distribution with PDI<0.6 at cholesterol concentrations of 10-40 wt %. The charge of the particles, as shown by the zeta potential data in FIG. 3C, varied from +12.0±5.86 mV for PF-LNPs without PP75 coating, to −41.7±3.71 mV for PF-LNPs with 1.3×10⁴ polymer chains/particles. The switch between positively charged to negatively charged occurred between 0.5×10⁴ and 1.0×10⁴ polymer chains/particle. The negative charge is beneficial for vaccine applications, as drainage into lymph nodes is more efficient (as evidenced in Example 4 described below), triggering high levels of antibody production¹¹. This is one of major advantages of this delivery system as cationic or ionisable LNPs with positive surface charge, which are widely used for nucleic acid delivery, can compromise the drainage efficiency into lymph nodes.

In Situ Loading of saRNA into PF-LNPs

The in situ loading of saRNA into the PF-LNPs was also investigated. saRNA (˜9500 nt) is large and highly negatively charged relative to other nucleic acids such as mRNA. Hence, it is important to optimize the delivery systems to allow for maximum encapsulation efficiency. FIG. 4A shows that the saRNA encapsulation efficiency of PF-LNPs coated with 1.0×10⁴ polymer chains/particle, as determined by a RiboGreen™ Assay, was dependent on the cholesterol content. As the cholesterol content increased, the encapsulation efficiency increased. The PF-LNPS at 40 wt % cholesterol produced the highest saRNA encapsulation. This could be due to the possibility that cholesterol changes the mechanism with which the polymer and particle interact; this in turn can affect the rigidity of the system⁹. Without cholesterol, FIG. 4A shows relatively low saRNA encapsulation efficiency. This may be due to the unstable hydrophobic side chains of PP75 in aqueous solution, which are embedded in the apolar region of the lipid membrane¹², and when cholesterol is introduced to the system, the lipid packing becomes more condensed¹³, making the membrane more rigid¹⁴. This in turn, reduces the permeability of the system¹⁵, allowing for higher encapsulation efficiencies and reduced leakage to be achieved at pH 7.4.

FIG. 4B shows the saRNA encapsulation efficiencies of the PF-LNPs (fixed cholesterol content of 40 wt %) coated with PP75 at 0.5×10⁴, 1.0×10⁴ and 1.3×10⁴ polymer chains/particle. The encapsulation efficiency peaked to 87.2±9.8% at the PP75 density of 0.5×10⁴ chains/particles, then decreased to 51.3±2.4% at the PP75 density of 1.3×10⁴ polymer chains/particles. There is a possibility that not all saRNA is encapsulated inside the membrane; some may be attached to the surface, and the interaction of this saRNA is worth considering. The reason for the reduction in encapsulation efficiency with increasing polymer density could be due to the presence of 40 wt % cholesterol, enhancing the hydrophobicity on the lipid membrane, causing the hydrophobic interaction of PP75 with the particle to be mostly at the surface rather than by translocation through the lipid membrane¹³. This increased charge density at the surface could conflict with the highly negatively charged saRNA via electrostatic repulsion on the surface of the particle, preventing some saRNA from binding to the surface.

Accordingly, it was found that using the DOTAP/DOPE ratio of 1:1, 40 wt % cholesterol, and 0.5×10⁴ PP75 polymer chains/particle produced a maximum encapsulation efficiency of 90% in the PF-LNPs.

### Cytotoxicity of PF-LNPs

The effect of the saRNA-encapsulated PF-LNPs on the metabolic activity of HeLa cervical cancer cells was investigated using Alamar Blue assay. FIG. 5A shows HeLa cell viability over 48 h when incubated with saRNA-encapsulated PF-LNPs (DOTAP/DOPE=1:1, 40 wt % cholesterol) coated with PP75 at 0.5×10⁴, 1.0×10⁴ and 1.3×10⁴ polymer chains/particle, as well as with saRNA only and saRNA-encapsulated LNPs without PP75 coating. Generally, the relative cell viability remained between 66.1±0.3% and 98.2±0.2% and it gradually decreased over time for all samples. The relative cell viability for saRNA remained above 95.8±1.00%.

At 24 h, the relative cell viability of the PF-LNPs without saRNA was generally slightly higher than the PF-LNPS with saRNA, as shown in FIG. 5B. Moreover, no significant change in the relative cell viability was observed when HeLa cells were treated with the PF-LNPs with PP75 coating density within the range tested, maintaining a high cell viability >80%, comparable with saRNA only. This suggests that the PF-LNPs had low or negligible cytotoxicity and the surface coating with the anionic endosomolytic amphiphilic polymers did not induce obvious cytotoxicity.

Damage to the plasma membrane integrity can be a good indication of significant injury prior to cell death and this can be determined by measuring the activity of lactate dehydrogenase (LDH), a stable cytosolic enzyme, in the extracellular medium. FIG. 5C shows the effect of the PF-LNPs, with and without saRNA, on the HeLa cell membrane integrity as determined using LDH assay. When the HeLa cells were incubated with saRNA only, there was a high relative cell viability at 91.4±4.5%, indicating that it does not cause significant membrane damage. As the polymer coating density increased, the relative cell viability gradually decreased, in the case of the PF-LNPs both with and without saRNA. Despite this, the HeLa cells generally tolerated the PF-LNPs well, with the lowest cell viabilities being 78.6±5.51% and 71.3±2.2% for PF-LNPs coated with 1.3×10⁴ PP75 polymer chains/particle, with and without saRNA encapsulation, respectively. This suggests that, after 24 h of incubation, the polymer coated on the PF-LNP surface may contribute to only limited membrane damage even at higher polymer coating densities.

pH-Responsive Membrane-Destabilizing Activity of PF-LNPs

Hemolysis assay was performed on the RBC membrane to model the endosomal membrane⁷ to examine the ability of the PF-LNPs to trigger endosomal escape. FIG. 6 shows the pH-responsive cell membrane destabilizing activity of PF-LNPs coated with 1.0×10⁴ and 1.3×10⁴ PP75 polymer chains/particle. It is obvious that, with a decrease from physiological pH to early endosomal pHs (7.0-6.0), the relative haemolysis increased considerably. Just as these results show, an effective cytoplasmic delivery system should have low haemolysis at physiological pH and the ability to efficiently destabilize the membrane of endosomes, preferably early endosomes, when acidification in the endosomal compartments takes place⁹.

Moreover, the relative haemolysis increased as the polymer coating density increased, with the highest being saRNA-encapsulated PF-LNPs coated with 1.3×10⁴ PP75 polymer chains/particle, reaching 77.9±6.6% at early endosomal pH 6.0, as shown in FIG. 6A. These results were comparable with the hemolysis of the PF-LNPs without saRNA, shown in FIG. 6B. This suggests that PP75 coated on the PF-LNP surface has the ability to trigger endosomolytic activity at early endosomal pHs, making the PF-LNPs a suitable candidate for efficient intracellular delivery of macromolecular payload including RNA.

### Effect of Lipid Compositions and Polymer Coating Densities on In Vitro HEK-293 Cell Transfection by PF-LNPs

fLuc-encoding saRNA was encapsulated into the PF-LNPs coated with PP75 at 0.5×104, 1.0×10⁴ and 1.3×10⁴ polymer chains/particle. The PF-LNPs with DOTAP/DOPE at a weight ratio of 1:1 (FIG. 7A), 1:2 (FIG. 7B) and 1:4 (FIG. 7C), were transfected into human embryonic kidney 293 (HEK-293) cells to determine the cell transfection efficiency.

FIG. 7A shows that the transfection efficiency of all formulations of PF-LNPs comprising DOTAP/DOPE at a weight ratio of 1:1 and 40 wt % cholesterol was at least 0.5 order of magnitude higher than the commercially available gold standard, polyethylenimine (PEI). The highest transfection efficiency was achieved by PF-LNPs coated with PP75 at 1.0×10⁴ and 1.3×10⁴ polymer chains/particles, reaching 10⁸ RLU which was 1.5 orders of magnitude higher compared to PEI. Overall, using the DOTAP/DOPE weight ratio of 1:1 produced the highest transfection efficiencies compared to DOTAP/DOPE (1:2) and DOTAP/DOPE (1:4) based PF-LNPs.

### Effect of N/P Ratio on In Vitro HEK-293 Cell Transfection by PF-LNPs

Furthermore, it was important to investigate the effect of the N/P ratio of lipid to saRNA on the transfection efficiency of the PF-LNPs with different PP75 coating densities. FIG. 8 compares N/P ratio of 0.1, 1 and 10. It was found that, as the N/P ratio increased, transfection efficiency increased. The transfection efficiency was the highest in the formulations with the N/P ratio of 10, the highest being the PF-LNPs with 1.0×10⁴ and 1.3×10⁴ polymer chains/particles. Although the difference in transfection efficiency of N/P ratio 1 and 10 was statistically significant, N/P ratio 1 was selected for in vivo studies.

### EXAMPLE 2—IN VITRO TRANSFECTION IN INTERFERON-COMPETENT HELA CELLS BY PF-LNPS

In addition to inefficient protein expression, clinical applications of RNA systems are restricted by their high innate immunogenicity. The RNA delivery using conventional cationic or ionizable LNPs with positive surface charge may induce protective immune responses. Therefore, it is crucial to achieve a good balance between protein expression and innate immune response. FIG. 9 shows a cell transfection titre of PF-LNPs on interferon-competent HeLa cells. As the dose of saRNA increased, the transfection efficiency increased substantially up to ˜10⁷ RLU with 100 ng/well. However, at 1000 ng/well, transfection efficiency decreased to ˜10⁴⁻⁵ RLU, which could be due the induced protective immune responses at high saRNA dose. It is noteworthy that delivery of the same saRNA dose of 100 ng/well using the same PF-LNPs formulation can result in high transfection efficiency in the interferon-competent HeLa cells (FIG. 9), comparable with that in the HEK-239 cells (FIG. 8). These results suggest that the PF-LNPs can perform well on both criteria of protein expression and innate immunogenicity. This is attributed to functionalization of the positively charged LNP surface with anionic pH-responsive endosmolytic pseudopeptides mimicking anionic fusogenic peptides in the hemagglutinin spikes of influenza virus, which can result in the negative surface charge of PF-LNPs (FIG. 3C) and prevent direct interactions of cationic/ionisable lipids with cells, leading to high transfection efficiency in interferon-competent cells

These results also demonstrate the efficient delivery into various cell types including cancer cells, showing the versatility of PF-LNPs for vaccine and gene therapy applications and other therapeutics.

The inventors noted that, overall, PF-LNPs have great potential to work well in in vivo studies, given that the transfection efficiencies are much higher than the commercially available gold standard for intracellular RNA delivery.

### EXAMPLE 3—ELUCIDATION OF THE ENDOCYTIC MECHANISM OF PF-LNPS

It is crucial to design a nano-carrier which can release endocytosed biological molecules into the cytoplasm by endosomal escape before they are trafficked to lysosomes for degradation (FIG. 1). The endocytic mechanism of intracellular delivery of the PF-LNPs was investigated by using pathway inhibitors at the point of delivery into cells. Six different pathway inhibitors were used, each inhibits a specific cellular trafficking pathway. The cellular uptake of saRNA encoding enhanced green fluorescent protein (EGFP-saRNA) encapsulated PF-LNPs was visualized in the confocal images (FIG. 10A) and quantified by flow cytometry (FIG. 10B). As expected, strong green fluorescence was observed in HEK-293 cells treated with EGFP-saRNA encapsulated PF-LNPs only, further conforming the efficient protein expression in cells. It is clear that when methyl-β-cyclodextrin (MβCD) was used in combination with the EGFP-saRNA encapsulated PF-LNPs, there was the lowest EGFP expression inside cells, with the green fluorescence intensity decreased by 80%). This means there was very little cellular uptake of the PF-LNPs as a result of treatment of cells with the inhibitor MβCD, suggesting that lipid-raft mediated endocytosis is the main cellular uptake pathway of PF-LNPs.

This finding shows the mechanistic pathway PF-LNPs take to enter cells. Knowing this helps us have a better understanding of how the delivery system works to determine which payloads would be most suitable for delivery using PF-LNPs. It also allows us to predict the behaviour of PF-LNPs under certain conditions.

The results of this study show that any payload that needs to enter cells via the most common intracellular delivery pathway, the clathrin-dependent endocytic pathway, can do so successfully with our delivery system.

### EXAMPLE 4—IN VIVO STUDIES SHOWING EFFICIENT PROTEIN EXPRESSION AND EXCELLENT IMMUNOGENICITY EFFECTS IN MOUSE MODELS, AND IMPROVED LYMPH NODE DRAINAGE THANKS TO FAVOURABLE SURFACE CHEMISTRY OF PF-LNPS

It is important to determine whether the PF-LNPs developed in Example 1 are capable of delivering saRNA in vivo, considering the high transfection efficiencies achieved in-vitro in HEK-293 (FIGS. 7 and 8) and interferon-competent HeLa cells (FIG. 9), and the fact that in vitro results do not always translate in vivo¹⁶.

FIG. 11 shows that the polymer coating density on the PF-LNPs played a significant role in producing a higher luciferase expression in mice. As the PP75 coating density increased, the luciferase expression increased remarkably and was relatively more dispersed. These images further reinforce that the PF-LNPs play a major role in more efficient intracellular delivery of nucleic acids in vivo.

The efficient in vivo luciferase expression led to an in vivo immunogenicity study of the different PF-LNP formulations to demonstrate the potential for this delivery system to be used in RNA vaccines. Female BALB/c mice were injected with hemagglutinin influenza virus encoded saRNA (HA-saRNA), encapsulated in PF-LNPs with 1.0×10⁴ and 1.3×10⁴ PP75 polymer chains/particle. Naïve, HA-saRNA complexed with jetPEI, and HA-saRNA encapsulated LNPs without PP75 coating were also tested as controls. 1 μg HA-saRNA was encapsulated in each formulation, except for the PF-LNP formulation coated with PP75 at 1.3×10⁴ polymer chains/particle where a 0.1 μg dose was used additionally. Mice received the first injection followed by a second dose after 4 weeks. HA IgG antibody titers were quantified at week 4, prior to administration of the second dose, and at week 6, two weeks after the second dose (FIG. 12B). The percentage survival (FIG. 12A) and body weight (FIG. 12C) were also recorded at the end of each week of the study.

As shown in FIG. 12B, mice treated with naïve HA-saRNA did not survive past day 5 after the influenza challenge was introduced, probably as a result of the sudden weight loss observed in FIG. 12C, due to degradation of saRNA by RNAase¹⁷. Mice treated with HA-saRNA encapsulated LNPs without PP75 coating only had 40% survival by day 6, and sudden weight loss was observed by day 5. This is reflected in the antibody titers (FIG. 12A), showing relatively low antibody expression. The negative surface charge of the PF-LNPs (FIG. 3B) could explain this; negative charge allows the delivery system to drain more easily into lymph nodes, resulting in a higher immune response¹¹. Without the PP75 coating, the overall charge of the PF-LNPs is positive (FIG. 3B), resulting in lower antibody expression. Interestingly, the remaining 40% of mice experienced an increase in body weight post-day 5, suggesting that the second dose at week 4 might have a therapeutic effect on the remaining mice. All other groups had 100% survival and maintained healthy weight change. Looking more closely at the antibody titers (FIG. 12A), it is clear that the PF-LNPs produced high antibody levels, on par with jetPEI, the gold standard, by week 4 (˜104 ng/mL). Furthermore, the group given a lower dose of 0.1 μg HA-saRNA formulated in PF-LNPs coated with PP75 at 1.3×10⁴ polymer chains/particle also produced promising results. The antibody expression was on par with jetPEI complexed with 1 μg HA-saRNA, although a slightly lower body weight percentage was observed. This suggests that the PF-LNPs have the ability to trigger a similar immune response at lower doses of HA-saRNA, and overall, the anionic, viral-peptide-mimicking, endosomolytic polymer coated on the PF-LNP surface plays a significant role in making this happen, which is a major advantage in the field of RNA delivery.

The next logical step was to confirm the hypothesis that surface functionalization of the LNPs with anionic, biomimetic, amphiphilic polymers results in enhanced drainage of RNA into lymph nodes, resulting in higher protein expression. Lymph node drainage in mouse model was investigated by comparing EGFP-saRNA only (FIG. 13A) to EGFP-saRNA encapsulated PF-LNPs coated with Cy5-PP75 at 1.0×10⁴ polymer chains/particle (FIG. 13B) and at 1.3×10⁴ polymer chains/particle (FIG. 13C). The pink regions of the widefield microscope image represent fatty tissue of the lymph node and blue region represent nuclei. Importantly, the fluorescence images show the substantially stronger green fluorescence for the EGFP-saRNA encapsulated PF-LNPs, relative to EGFP-saRNA only, and a significant increase in green fluorescence when the PP75 coating density was increased. This suggests that surface functionalization of the nanoparticle with anionic amphiphilic polymers does enhance the drainage of the PF-LNPs into lymph nodes, which is extremely advantageous for vaccine applications, where triggering protein expression is vital.

### EXAMPLE 5— CO-DELIVERY OF RNA AND SMALL MOLECULES USING PF-LNPS

Co-Delivery of saRNA and Ruxolitinib to MRC-5 Cells

The co-delivery of saRNA with a targeted therapy drug Ruxolitinib, a Janus Associated Kinase (JAK) inhibitor with selectivity for subtypes JAK1 and JAK2, using PF-LNPs coated with PP75 at 1.3×10⁴ polymer chains/particle was investigated to explore the effect of introducing small-molecule drugs on the encapsulation efficiency and cell transfection efficiency of macromolecules such as nucleic acids. FIG. 14 shows that as the Ruxolitinib concentration increased from zero to 0.02 μg/mL, the saRNA encapsulation efficiency increased from 69.0±3.91% to 73.5±11.6%. However, a further increase in the Ruxolitinib concentration resulted in a reduction in the saRNA encapsulation efficiency, which could be due to the limited space available for encapsulation as higher amounts of small-molecule drug filling the voids alongside saRNA. To our knowledge, co-delivery of small-molecule drugs with saRNA has not yet been achieved possibly due to stability issues as a result of the highly negative charge of saRNA. This further supports the stability and robustness of the versatile PF-LNPs.

Cell transfection efficiency into MRC-5 cells, human embryonic lung fibroblasts, was investigated using fLuc assay. As shown in FIG. 15, as the concentration of Ruxolitinib increased, the transfection efficiency of the saRNA-encapsulated PF-LNPs also increased. The PF-LNPs, comprising DOTAP/DOPE (1:1), 40 wt % cholesterol, 1.3×10⁴ PP75 polymer chains/particle and 1 μg/mL Ruxolitinib, produced a maximum transfection efficiency, −2 orders of magnitude higher than commercially available PEI. This shows that, introducing Ruxolitinib to the system can enhance the transfection efficiency of saRNA, considerably higher than the gold standard, PEI, without compromising the stability of the system.

Co-Delivery of saRNA and Trehalose to HEK-293 Cells

Co-delivery of trehalose, a protectant that can stabilize biomolecules during freezing and drying, was investigated to assess the potential for stable storage of the RNA-encapsulated PF-LNPs, both lyophilized and in aqueous solution, at room and tropical temperatures without compromising the integrity of the lipid structure or functionality of the payload.

FIG. 16 demonstrates the characterization and successful HEK-293 cell transfection of the formulations, both in aqueous solution and lyophilized, of saRNA-encapsulated PF-LNPs coated with PP75 at 1.3×10⁴ polymer chains/particle, with co-existence of trehalose both inside and outside PF-LNPs. FIG. 16A shows that using trehalose concentrations of 63 mg/mL or above ensured particle sizes remained below 200 nm and PDI values were less than 0.6 for all the PF-LNP formulations. FIG. 16B shows the zeta potential became more negative as a higher concentration of trehalose was used, the lowest being at −57.3±3.6 mV for lyophilized PF-LNP containing 500 mg/mL trehalose. Moreover, the lyophilized formulations had a more negative zeta potential compared to the respective formulations in aqueous solution. HEK-293 cell transfection shown in FIG. 16C demonstrated a synergistic effect of trehalose on the cell transfection efficiency. As the trehalose concentration increased, transfection efficiency increased, reaching as high as 108 RLU at 250 and 500 mg/mL trehalose. This suggests that co-existence of trehalose both inside and outside the PF-LNPs was able to maintain the high efficacy of the saRNA, in aqueous solution and even after lyophilization.

However, there is a threshold to how much trehalose can be included in the PF-LNP formulations. Trehalose concentrations as high as 500 mg/mL can exert some cytotoxic effect for such delivery systems, as demonstrated in FIG. 18, which compared the viability of HEK-293 cells when treated with the PF-LNP formulations, in aqueous solution (FIG. 17A) and lyophilized (FIG. 17B), containing trehalose at various concentrations. The lyophilized formulation with 500 mg/mL trehalose caused the lowest relative cell viability, at 53.3±8.0% after 48 h of incubation (FIG. 17B). Therefore, a concentration of 250 mg/mL was used for the long-term storage studies due to the high negative surface charge and high transfection efficiency that resulted from using it.

### EXAMPLE 6—CO-ENCAPSULATION OF STABILISING MOLECULES IN THE PF-LNP FORMULATIONS, BOTH IN AQUEOUS SOLUTION AND LYOPHILIZED, FOR LONG-TERM STABLE STORAGE OF RNA AT AMBIENT TEMPERATURES

After optimising the PF-LNPs with trehalose co-encapsulation, a long-term storage study was carried out to measure the particle size, surface charge, HEK-293 cell viability and HEK-293 cell transfection of the PF-LNPs co-encapsulated with saRNA and trehalose, over time at different temperatures. Four storage temperatures of −20° C., 4° C., 20° C. and 40° C. were used to mimic freezer, fridge, room and tropical conditions. FIG. 18 demonstrates the interactions of trehalose with RNA-encapsulated PF-LNPs, both in aqueous solution and lyophilized. Trehalose is present both inside and outside the PF-LNPs to offer protection from the both sides, ensuring the desirable physical stability (e.g., prevention of particle aggregation and payload leakage) and chemical stability (e.g., protection of biomolecules such as RNA) of the nanoparticle during manufacturing and storage, thus maintaining and prolonging the functionality of saRNA.

FIG. 19 shows the characterization of the formulations, in aqueous solution and lyophilized, of saRNA-encapsulated PF-LNPs coated with PP75 at 1.3×10⁴ polymer chains/particle, with co-existence of 250 mg/mL trehalose both inside and outside PF-LNPs (PF-LNPs/tre). When fresh, the particle size remained below 200 nm and PDI was less than 0.5. This was the case for both PF-LNPs/tre and uncoated LNPs (LNPs/tre). After storage at −20° C. for 52 weeks, both PF-LNPs/tre and LNPs-tre maintained similar hydrodynamic sizes compared to the fresh formulations, whist their PDI values increased in Week 16 with a more significant change for LNPs/tre and then further increased up to ˜1.0 in Week 52. At 4° C. over 52 weeks, PF-LNPs/tre remained below 200 nm in size and below ˜0.8 in PDI. However, LNPs/tre increased in size to 200-300 nm and PDI of 1 after the extended storage, suggesting the particles might have aggregated. Over the 52-week period, the hydrodynamic size and PDI increased for both PF-LNPs/tre and LNPs/tre at 20° C. and more significantly at 40° C. Overall, the PF-LNPs/tre shows stability in hydrodynamic size and PDI more than LNPs/tre over 52 weeks.

FIG. 20 show the zeta potential of PF-LNPs/tre and LNPs/tre, in aqueous solution and lyophilized, over 52 weeks. Fresh PF-LNPs/tre and LNPs/tre in solution had a zeta potential of −57.8±1.5 mV and +10.4±4.5 mV, respectively. The zeta potential of freshly lyophilized PF-LNPs/tre and LNPs/tre was −48.3*1.8 mV and +18.8±3.8 mV, respectively. The zeta potentials at all temperatures of −20° C., 4° C., 20° C. and 40° C. remained consistent throughout the 52-week storage for PF-LNPs/tre, both in aqueous solution and lyophilized, with very small variance in comparison to the fresh formulations. However, the zeta potentials of LNPs/tre changed from positive to negative over time at all temperatures. This is most likely because of leakage of the highly negatively charged saRNA over time. This suggests that PF-LNPs displayed higher stability compared to LNPs, due to surface functionalization with anionic, viral-peptide-mimicking, amphiphilic polymers.

FIG. 21 shows the HEK-293 cell transfection efficiency of PF-LNPs/tre and LNPs/tre, in aqueous solution and lyophilized, over 52 weeks of storage. Fresh PF-LNPs/tre and LNPs/tre in solution and lyophilized showed transfection efficiency of ˜107 RLU and ˜10⁶ RLU, respectively. The transfection efficiency of the lyophilized PF-LNPs/tre after 52 weeks of storage at all temperatures of −20° C., 4° C., 20° C. and 40° C., respectively, remained consistent at a high level, generally one order of magnitude higher than LNPs/tre, throughout the 52-week period. This has validated that the polymer functionalization and coexistence of trehalose both inside and outside nanoparticles can successfully preserve the functionality of saRNA. RNA molecules are very fragile and may readily degrade in exposure environments, thus requiring storage and distribution in a very challenging ultra-cold or cold chain. For example, the Pfizer/BioNTech mRNA vaccine, the world's first approved COVID-19 vaccine, is plagued by the major hurdle requiring storage at −70° C., while at refrigerated temperatures of 2-8° C. it can be stable for only 5 days (Pfizer.com, 20 Nov. 2020). Similarly, the approved Moderna mRNA vaccine against COVID-19 needs to be held in storage at −20° C. The excellent thermal stability of RNA and high transfection efficiency even at room (20° C.) and tropical (40° C.) temperatures over one year for the PF-LNP formulations, which the inventors have demonstrated, represent an important technological breakthrough in manufacturing, storage and distribution of RNA vaccines and biotherapeutics and have tremendous commercial potential and socioeconomic impact.

As also shown in FIG. 21, PF-LNPs/tre in aqueous solution during 52 weeks of storage at −20° C., 4° C., 20° C. and 40° C. retained good transfection efficiencies, although approximately one order of magnitude lower compared with lyophilised PF-LNPs/tre. In Week 52, the transfection efficiency of PF-LNPs/tre in aqueous solution still remained >10⁴ RLU for storage at −20° C., 4° C., 20° C. and >10³ RLU for storage at 40° C. These are significant findings considering that it is a major challenge to store RNA in aqueous solution since the RNA molecule can be readily degraded through hydrolysis. Favorable thermal stability of RNA vaccines and biotherapeutics in aqueous solution at ambient temperatures through formulation in the PF-LNPs provides another attractive strategy, alternative to lyophilized formulations, for stable RNA storage without need for an ultra-cold or cold chain. In addition, it is worth pointing out that: (i) the N/P ratio of 1 was used in this preliminary storage work and the use of higher N/P ratios will enhance transfection efficiencies (as shown in FIG. 8); (ii) PF-LNP compositions and formulations can be further optimized. The inventors believe that thermal stability of RNA formulated in the PF-LNPs after extended storage at ambient temperatures could be further enhanced after optimization.

FIG. 22 shows that the relative viability of HEK-293, as determined using Alamar Blue assay, remained above ˜70% after 48 h of treatment with all the formulations of PF-LNPs, both in aqueous solution and lyophilized, throughout the 52-week storage period at all temperatures of −20° C., 4° C., 20° C. and 40° C., respectively. Those HEK-293 cell viabilities of PF-LNP/tre and LNP/tre were comparable with the negative control saRNA/tre, the mixture of saRNA and trehalose, further confirming that the PF-LNP formulations had negligible or low cytotoxicity.

### EXAMPLE 7—PREPARATION & CHARACTERIZATION OF PEGYLATED PF-LNPS, HIGH BIOCOMPATIBILITY & LOW CYTOTOXICITY, AND HIGH IN VITRO TRANSFECTION EFFICIENCY VIA EFFICIENT INTRACELLULAR RNA DELIVERY

The inventors synthesised PEGylated polymer functionalized lipid nanoparticles (PEGylated PF-LNPs) as explained in the Materials and Methods section below. The PEGylated PF-LNPs were coated with the biocompatible, pH-responsive, endosomolytic pseudopeptidic polymer, PP75, which mimics the anionic fusogenic peptides in the hemagglutinin spikes of influenza virus.

### Physiochemical Characterisation of PEGylated PF-LNPs

FIG. 23 shows the effect of polymer density on the hydrodynamic diameter, PDI and zeta potential of the PEGylated PF-LNPs, in aqueous solution and lyophilised form. In aqueous solution, the average particle size shown in FIG. 23A was found to be similar at 112.0±69.0 nm for the PF-LNPs coated with PP75 at 0.25 mg/mL, 0.5 mg/mL and 1 mg/mL. PDI was also similar at ˜0.2, suggesting the PEGylated PF-LNPs were monodispersed. In comparison, the lyophilised formulations showed a slightly higher average hydrodynamic diameter at 128±23.0 nm and PDI˜0.4. However, these monodisperse particles were still under 200 nm for effective intracellular delivery. The charge of the particles, as shown by the zeta potentials in FIG. 23B, varied from +15.9±0.5 mV for PEGylated LNPs without PP75 coating, to −55.4±0.3 mV for PEGylated PF-LNPs with 1 mg/mL PP75. Both aqueous solutions and lyophilized formulations followed the same trend. The switch between positively charged to negatively charged occurred between 0.25 mg/mL and 0.5 mg/mL. As mentioned previously, negative charge is beneficial for vaccine applications, as drainage into lymph nodes is more efficient (as evidenced in Example 4), triggering high levels of antibody production¹¹. This is one of major advantages of these delivery systems.

In Situ Loading of Calcein, FITC-Dextran and saRNA into PEGylated PF-LNPs

The in situ loading of model payloads, Calcein (Mw˜622.55 Da) and FITC-Dextran (Mw˜150 kDa) into the PF-LNPs coated with 1 mg/mL PP75 was investigated to demonstrate the ability of the PEGylated-PF-LNPs to encapsulate a wide size range of payloads. FIG. 24A shows the encapsulation efficiency of Calcein and FITC-Dextran in PEGylated LNPs was 80.9±1.8% and 76.9±2.4%, respectively. FIG. 24B shows that the saRNA encapsulation efficiency of PEGylated PF-LNPs coated with 1 mg/mL PP75, as determined by a RiboGreen™ Assay, was 84.91±2.32%. This is as expected for passively loaded payloads.

### EXAMPLE 8—CO-ENCAPSULATION OF STABILISING MOLECULES IN THE PEGYLATED PF-LNP FORMULATIONS, BOTH IN AQUEOUS SOLUTION AND LYOPHILIZED, FOR LONG-TERM STABLE STORAGE OF RNA AT AMBIENT TEMPERATURES

After optimising the PEGylated PF-LNPs with trehalose co-encapsulation, a long-term storage study was carried out to measure the particle size, surface charge, HEK-293 cell viability and HEK-293 cell transfection of the PEGylated PF-LNPs co-encapsulated with saRNA and trehalose, over time at different temperatures. Four storage temperatures of −20° C., 4° C., 20° C. and 40° C. were used to mimic freezer, fridge, room and tropical conditions. Trehalose is present both inside and outside the PF-LNPs to offer protection from the both sides, ensuring the desirable physical stability (e.g., prevention of particle aggregation and payload leakage) and chemical stability (e.g., protection of biomolecules such as RNA) of the nanoparticle during manufacturing and storage, thus maintaining and prolonging the functionality of saRNA.

FIG. 25 shows the characterization of the formulations, in aqueous solution [A] and lyophilized [B], of saRNA-encapsulated PEGylated PF-LNPs coated with PP75 at 1 mg/mL, with co-existence of 250 mg/mL trehalose both inside and outside PEGylated PF-LNPs (PEGylated PF-LNPs/tre). When fresh, the particle size remained below 150 nm and PDI was less than 0.4. This was the case for both PEGylated PF-LNPs/tre and uncoated PEGylated LNPs (PEGylated LNPs/tre). After storage at −20° C., 4° C., 20° C. and 40° C. for 9 weeks, both PEGylated PF-LNPs/tre and PEGylated LNPs/tre maintained similar hydrodynamic sizes and PDI compared to the fresh formulations. Overall, both the PEGylated PF-LNPs/tre and PEGylated LNPs/tre showed stability in hydrodynamic size and PDI more over 9 weeks, in aqueous solution and lyophilized form.

FIG. 26 shows the zeta potential of PEGylated PF-LNPs/tre and PEGylated LNPs/tre, in aqueous solution [A] and lyophilized [B], over 9 weeks. Fresh PF-LNPs/tre and LNPs/tre in solution had a zeta potential of −55.4±0.3 mV and +15.9±0.5 mV, respectively. The zeta potential of freshly lyophilized PEGylated PF-LNPs/tre and PEGylated LNPs/tre was −52.1±1.6 mV and +10.5±5.2 mV, respectively. The zeta potentials at all temperatures of −20° C., 4° C., 20° C. and 40° C. remained consistent throughout the 9-week storage for both PEGylated PF-LNPs/tre and PEGylated LNPs/tre, in aqueous solution and lyophilized form, with very small variance in comparison to the fresh formulations.

FIG. 27 shows the HEK-293 cell transfection efficiency of PEGylated PF-LNPs/tre and PEGylated LNPs/tre, in aqueous solution and lyophilized, over 9 weeks of storage. Fresh PEGylated PF-LNPs/tre and PEGylated LNPs/tre in solution and lyophilized showed transfection efficiency of ˜10⁷ RLU and ˜10⁶ RLU, respectively. The transfection efficiency of the lyophilized PEGylated PF-LNPs/tre and PEGylated LNPs/tre after 9 weeks of storage at all temperatures of −20° C., 4° C., 20° C. and 40° C., respectively, remained consistent throughout with small variance in comparison to the fresh formulations, although the PEGylated LNPs/tre transfection efficiency generally remained one order of magnitude below PEGylated PF-LNPs/tre throughout the 9-week period. This has validated that, for both PEGylated PF-LNPs with polymer functionalization and PEGylated LNPs without polymers, coexistence of trehalose both inside and outside nanoparticles can successfully preserve the functionality of saRNA. The excellent thermal stability of RNA and high transfection efficiency even at room (20° C.) and tropical (40° C.) temperatures particularly for the PEGylated PF-LNP formulations, which the inventors have demonstrated, represent another important technological breakthrough in manufacturing, storage and distribution of RNA vaccines and biotherapeutics and have tremendous commercial potential and socioeconomic impact.

Also shown in FIG. 27, the HEK-293 transfection efficiency of PEGylated PF-LNPs/tre and PEGylated LNPs/tre in aqueous solution after 9 weeks of storage at −20° C., 4° C., 20° C. and 40° C. remained consistent throughout with small variance in comparison to the fresh formulations. The PEGylated LNPs/tre transfection efficiency generally remained one order of magnitude below PEGylated PF-LNPs/tre throughout the 9-week storage in aqueous solution at all those temperatures tested. These are significant findings considering that it is a major challenge to store RNA in aqueous solution since the RNA molecule can be readily degraded through hydrolysis. In addition, it is worth pointing out that: (i) the N/P ratio of 1 was used in this preliminary storage work and the use of higher N/P ratios will enhance transfection efficiencies (as shown in FIG. 8 with PF-LNPs); (ii) PEGylated PF-LNP compositions and formulations can be further optimized. The inventors believe that thermal stability of RNA formulated in the PF-LNPs after extended storage at ambient temperatures could be further enhanced after optimization.

FIG. 28 shows that the relative viability of HEK-293, as determined using Alamar Blue assay, remained above ˜00% after 48 h of treatment with all the formulations of PEGylated PF-LNPs and PEGylated LNPs, both in aqueous solution and lyophilized, throughout the 9-week storage period at all temperatures of −20° C., 4° C., 20° C. and 40° C., respectively. These HEK-293 cell viabilities of PEGylated PF-LNPs and PEGylated LNPs were comparable with the negative control saRNA+tre, the mixture of saRNA and trehalose, further confirming that the PF-LNP formulations had negligible or low cytotoxicity.

### EXAMPLE 9—PREPARATION & CHARACTERIZATION OF MC3 PF-LNPS, HIGH BIOCOMPATIBILITY & LOW CYTOTOXICITY, AND HIGH IN VITRO TRANSFECTION EFFICIENCY VIA EFFICIENT INTRACELLULAR RNA DELIVERY

The inventors synthesised polymer functionalized lipid nanoparticles based on D-Lin-MC3-DMA ionizable lipid (MC3 PF-LNPs) as explained in the Materials and Methods section below. The MC3 PF-LNPs were coated with three different biocompatible, pH-responsive, endosomolytic pseudopeptidic polymers, PP75, PLP-NDA and PLP-ADA, which mimic the anionic fusogenic peptides in the hemagglutinin spikes of influenza virus.

PP75 is a pseudopeptide prepared by grafting hydrophobic amino acid L-phenylalanine to carboxylic acids pendant to the backbone of a metabolite-derived linear polyamide, poly(L-lysine iso-phthalamide) (PLP), at 75% stoichiometric degree of substitution. The biomimetic anionic amphiphilic polymer contains hydrophobic pendant groups and ionizable carboxylic acid groups found in the fusogenic peptides of influenza virus. PLP-NDA is a pH-responsive, comb-like polymer that displays excellent membrane anchoring and disruptive capabilities compared to PLP. The PLP-NDA polymer used in this work is synthesised by grafting the hydrophobic decylamine (NDA), which acts as a membrane anchor, onto the pendant carboxylic acid groups of PLP with ˜18% degree of grafting. This polymer has been shown to display excellent membrane disruption capabilities at endosomal pH and like PP75, no membrane disruption occurs at physiological pH (pH 7.4).

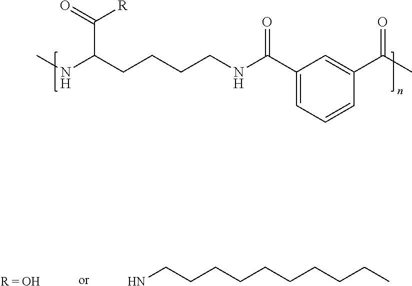

### Chemical Structure of PLP-NDA

PLP-ADA is a pH-responsive, comb-like polymers grafted with 12-aminododecanoic acid (ADA) as hydrophobic side chains on the PLP backbone for optimal intracellular delivery. The PLP-ADA polymer used in this work contains 60% degree of grafting with ADA.

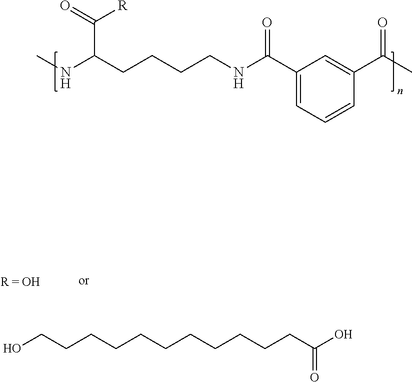

### Chemical Structure of PLP-ADA

### Physiochemical Characterisation of MC3 PF-LNPs

FIG. 29 shows the effect of PP75, PLP-NDA, and PLP-ADA polymer density on the hydrodynamic diameter, PDI and zeta potential of the MC3 PF-LNPs. The average particle size shown in FIG. 29A was found to be similar at ˜150 nm for the MC3 PF-LNPs coated with PP75 at 0.5 mg/mL and 1 mg/mL. PDI of the PP75-coated formulations was also similar at ˜0.3, suggesting the MC3 PF-LNPs were monodispersed. The uncoated MC3 LNPs had a PDI of 0.53±0.2, so they were more polydisperse than the polymer functionalized formulations. Similarly, the average particle size shown in FIG. 29B was found to be similar at ˜150 nm for the MC3 PF-LNPs coated with PLP-NDA at 0.5 mg/mL and 1 mg/mL. PDI of the PLP-NDA-coated formulations was also similar at ˜0.4, suggesting the MC3 PF-LNPs were monodispersed. In comparison, the average particle size shown in FIG. 29C was similar to the other two polymer coated PF-LNPs at ˜150 nm for the MC3 PF-LNPs coated with PLP-ADA at 0.5 mg/mL and it increased to ˜200 nm at 1 mg/mL. The PDI of PLP-ADA-coated formulations was ˜0.5 and ˜0.3 for PLP-ADA at 0.5 mg/mL and 1 mg/mL, respectively. This suggests PLP-ADA coated MC3 PF-LNPs were monodispersed. Also, all these monodisperse particles were under 200 nm, making them suitable for effective intracellular delivery. The charge of the particles, as shown by the zeta potentials in FIGS. 29A, B and C, varied from −1.6±0.5 mV for MC3 LNPs without polymer coating, to −8.8±0.6 mV for MC3 PF-LNPs with 1 mg/mL PP75, −22.3±0.2 mV for MC3 PF-LNPs with 1 mg/mL PLP-NDA, and −18.9±0.9 mV for MC3 PF-LNPs with 1 mg/mL PLP-ADA. All formulations followed the same trend; as the polymer concentration increased, the zeta potential decreased. As mentioned previously, the negative charge is beneficial for vaccine applications, due to enhanced drainage into lymph nodes (as evidenced in Example 4), leading to high antibody production¹¹. When encapsulated with saRNA, the hydrodynamic size and zeta potential of the MC3 PF-LNPs was not significantly affected, regardless of the type of polymer coating, as shown in FIG. 30.

In Situ Loading of Calcein, FITC-Dextran and saRNA into MC3 PF-LNPs

The in situ loading of model payloads, Calcein (Mw˜622.55 Da) and FITC-Dextran (Mw˜150 kDa) into the MC3 LNPs was investigated to demonstrate the ability of the MC3 LNPs to encapsulate a wide size range of payloads. FIG. 31A shows the encapsulation efficiency of Calcein and FITC-Dextran was 65.8±3.5% and 60.2 t 4.8%, respectively. FIG. 31B shows that the saRNA encapsulation efficiency in MC3 PF-LNPs, as determined by a RiboGreen™ Assay, was 66.82±1.54%. This is as expected for passively loaded payloads.

FIG. 32 shows HEK-293 cell transfection efficiency of PP75, PLP-NDA or PLP-ADA-coated MC3 PF-LNPs and MC3 LNPs. As shown, the polymer functionalization plays a significant role in enhancing the transfection efficiency of saRNA; as the polymer concentration increased, the transfection efficiency increased by at least one order of magnitude. The PP75 coating performed the best compared to the other polymers, with a transfection efficiency of ˜10^{7.5} RLU at 1 mg/mL of PP75.

FIG. 33 shows that the relative viability of HEK-293, as determined using Alamar Blue assay, remained above 80% after 48 h of treatment with all the formulations of MC3 PF-LNPs and MC3 LNPs. This confirmed that the PF-LNP formulations had negligible or low cytotoxicity.

FIG. 34 shows the pH-dependent size and surface charge change of MC3 LNPs. As the pH was increased, the hydrodynamic size of MC3 LNPs decreased, being the lowest at pH 7.4, 88.13*6.9 nm, as shown in FIG. 34A. Despite the higher hydrodynamic sizes at lower pH, the sizes remained under 200 nm, making them suitable for cellular uptake. The PDI of the formulation was variable, ranging from PDI 0.2 to PDI 1.0, suggesting polydispersity, however the polymer-functionalization would give stability to the nanoparticles. FIG. 34B showed a gradual decrease in zeta potential of the MC3 LNPs as the pH increased. This is as expected as this is the typical behaviour of ionizable lipid.

### EXAMPLE 10—CO-ENCAPSULATION OF STABILISING MOLECULES IN THE MC3 PF-LNP FORMULATIONS, BOTH IN AQUEOUS SOLUTION AND LYOPHILIZED, FOR LONG-TERM STABLE STORAGE OF RNA AT AMBIENT TEMPERATURES

After optimising the MC3 PF-LNPs with trehalose co-encapsulation, a long-term storage study was carried out to measure the particle size, surface charge, HEK-293 cell viability and HEK-293 cell transfection of the MC3 PF-LNPs co-encapsulated with saRNA and trehalose, over time at different temperatures. Three storage temperatures of −20° C., 4° C. and 20° C. were used to mimic freezer, fridge and ambient conditions. Trehalose is present both inside and outside the PF-LNPs to offer protection from both sides, ensuring the desirable physical stability (e.g., prevention of particle aggregation and payload leakage) and chemical stability (e.g., protection of biomolecules such as RNA) of the nanoparticle during manufacturing and storage, thus maintaining and prolonging the functionality of saRNA.

FIG. 35 shows the characterization of the formulations, in aqueous solution and lyophilized, of saRNA-encapsulated MC3 PF-LNPs coated with PP75, PLP-NDA or PLP-ADA at 1 mg/mL, with co-existence of 250 mg/mL trehalose both inside and outside MC3 PF-LNPs (MC3 PF-LNPs/tre). When fresh, the particle size remained below 150 nm and PDI was less than 0.4 for all three types of polymer-coated MC3 PF-LNPs. This was the case for both fresh MC3 PF-LNPs/tre and fresh uncoated MC3 LNPs (PEGylated LNPs/tre) at week 0. After storage at −20° C., 4° C. and 20° C. for 11 weeks, both MC3 PF-LNPs/tre maintained similar hydrodynamic sizes and PDI compared to the fresh formulations. The uncoated MC3 PF-LNPs/tre showed the most instability with the hydrodynamic size reaching as high as ˜250 nm and PDI 0.6 at all temperatures tested. MC3 PF-LNPs/tre and MC3 LNPs/tre in lyophilized form followed a similar trend to the formulations in aqueous solutions at the different temperatures. Overall, MC3 PF-LNPs/tre showed stability in hydrodynamic size and PDI for over 11 weeks, in aqueous solution and lyophilized form.

FIG. 36 shows the zeta potential of MC3 PF-LNPs/tre coated with PP75, PLP-NDA or PLP-ADA at 1 mg/mL, and MC3 LNPs/tre, in aqueous solution and lyophilized form, over 11 weeks. Fresh MC3 PF-LNPs/tre with PP75, PLP-NDA or PLP-ADA and MC3 LNPs/tre in solution had a zeta potential of −14.6±0.4 mV, −25.5±0.7 mV, −24.7±0.6 mV and −7.5±0.8 mV, respectively. The zeta potential of freshly lyophilized MC3 PF-LNPs/tre with PP75, PLP-NDA or PLP-ADA and MC3 LNPs/tre was −15.2±0.3 mV, −23.5±0.2 mV, −28.0±0.3 mV and −8.4±0.7 mV, respectively. The zeta potentials at all temperatures of −20° C., 4° C. and 20° C. remained consistent throughout the 1-week storage for both MC3 PF-LNPs/tre and MC3 LNPs/tre, in aqueous solution and lyophilized form, with very small variance in comparison to the fresh formulations.

FIG. 37 shows the HEK-293 cell transfection efficiency of MC3 PF-LNPs/tre with PP75, PLP-NDA or PLP-ADA at 1 mg/mL, and MC3 LNPs/tre, in aqueous solution and lyophilized form, over 11 weeks of storage. Fresh MC3 PF-LNPs/tre and MC3 LNPs/tre in solution and lyophilized form showed transfection efficiency of ˜10^{7.5} RLU and ˜10⁶ RLU, respectively. The transfection efficiency of the lyophilized MC3 PF-LNPs/tre and MC3 LNPs/tre after 11 weeks of storage at all temperatures of −20° C., 4° C. and 20° C., respectively, remained consistent throughout with small variance in comparison to the fresh formulations, although the MC3 LNPs/tre transfection efficiency generally remained 1-3 orders of magnitude below MC3 PF-LNPs/tre throughout the 1-week period (FIG. 37B). This has validated that, for both MC3 PF-LNPs with polymer functionalization and MC3 LNPs without polymers, coexistence of trehalose both inside and outside nanoparticles can successfully preserve the functionality of saRNA. The excellent thermal stability of RNA and high transfection efficiency even at room (20° C.) temperature particularly for the MC3 PF-LNP formulations, which the inventors have demonstrated, represent another important technological breakthrough in manufacturing, storage and distribution of RNA vaccines and biotherapeutics and have tremendous commercial potential and socioeconomic impact.

Also shown in FIG. 37A, the HEK-293 transfection efficiency of MC3 PF-LNPs/tre and MC3 LNPs/tre in aqueous solution after 11 weeks of storage at −20° C., 4° C. and 20° remained consistent throughout with small variance in comparison to the fresh formulations. The MC3 LNPs/tre transfection efficiency generally remained 1-3 orders of magnitude below MC3 PF-LNPs/tre throughout the 1-week storage in aqueous solution at all those temperatures tested. These are again, significant findings, considering that it is a major challenge to store RNA in aqueous solution. In addition, as with the previous formulations, it is worth pointing out that: (i) the N/P ratio of 1 was used in this preliminary storage work and the use of higher N/P ratios will enhance transfection efficiencies (as shown in FIG. 8 with PF-LNPs); (ii) MC3 PF-LNP compositions and formulations can be further optimized. The inventors believe that thermal stability of RNA formulated in the PF-LNPs after extended storage at ambient temperatures could be further enhanced after optimization.

FIG. 38 shows that the relative viability of HEK-293, as determined using Alamar Blue assay, remained above 80% after 48 h of treatment with all the formulations of MC3 PF-LNPs and MC3 LNPs, both in aqueous solution and lyophilized form, throughout the 1-week storage period at all temperatures of −20° C., 4° C. and 20° C., respectively. These HEK-293 cell viabilities of MC3 PF-LNPs and MC3 LNPs were comparable with the negative control saRNA+tre, the mixture of saRNA and trehalose, further confirming that the PF-LNP formulations had negligible or low cytotoxicity.

### EXAMPLE 11—PREPARATION & CHARACTERIZATION OF DODAP PF-LNPS, HIGH BIOCOMPATIBILITY & LOW CYTOTOXICITY, AND HIGH IN VITRO TRANSFECTION EFFICIENCY VIA EFFICIENT INTRACELLULAR RNA DELIVERY

The inventors synthesised polymer functionalized lipid nanoparticles based on (1,2-dioleoyl-3-dimethylammonium-propane) ionizable cationic lipid (DODAP PF-LNPs) as explained in the Materials and Methods section below. The DODAP PF-LNPs were coated with the three different pseudopeptidic polymers, PP75, PLP-NDA and PLP-ADA.

### Physiochemical Characterisation of DODAP PF-LNPs

FIG. 39 shows the effect of PP75, PLP-NDA, and PLP-ADA polymer density on the hydrodynamic diameter, PDI and zeta potential of the DODAP PF-LNPs. The average particle size shown in FIG. 39A was found to be similar at ˜150 nm for the DODAP PF-LNPs coated with PP75 at 0.5 mg/mL and 1 mg/mL. PDI of the PP75-coated formulations was also similar at ˜0.2-0.4, suggesting the MC3 PF-LNPs were monodispersed. The uncoated DODAP LNPs had a PDI of 0.22±0.1, so they were similarly monodispersed to the polymer functionalized formulations. Similarly, the average particle size shown in FIG. 39B was found to be similar at ˜150 nm for the DODAP PF-LNPs coated with PLP-NDA at 0.5 mg/mL and 1 mg/mL. PDI of the PLP-NDA-coated formulations was also similar at ˜0.3, suggesting the DODAP PF-LNPs were monodispersed. In comparison, the average particle size shown in FIG. 39C was similar to the other two polymer coated PF-LNPs at ˜150 nm for the DODAP PF-LNPs coated with PLP-ADA at 0.5 mg/mL and 1 mg/mL. The PDI of PLP-ADA-coated formulations was ˜0.4 for PLP-ADA at 0.5 mg/mL and 1 mg/mL. This suggests PLP-ADA coated DODAP PF-LNPs were monodispersed. Also, all these monodisperse particles were under 200 nm, making them suitable for effective intracellular delivery. The charge of the particles, as shown by the zeta potentials in FIGS. 39A, B and C, varied from −7.3±0.4 mV for DODAP LNPs without polymer coating, to −15.7±0.2 mV for DODAP PF-LNPs with 1 mg/mL PP75, −26.9±1.6 mV for DODAP PF-LNPs with 1 mg/mL PLP-NDA, and −27.9±0.3 mV for DODAP PF-LNPs with 1 mg/mL PLP-ADA. All formulations followed the same trend; as the polymer concentration increased, the zeta potential decreased. As mentioned previously, the negative charge is beneficial for vaccine applications, due to enhanced drainage into lymph nodes (as evidenced in Example 4), leading to high antibody production¹¹. When encapsulated with saRNA, the hydrodynamic size and zeta potential of the DODAP PF-LNPs was not significantly affected, regardless of the type of polymer coating, as shown in FIG. 40.

In Situ Loading of Calcein, FITC-Dextran and saRNA into DODAP PF-LNPs

The in situ loading of model payloads, Calcein (Mw˜622.55 Da) and FITC-Dextran (Mw˜150 kDa) into the DODAP LNPs was investigated to demonstrate the ability of the DODAP LNPs to encapsulate a wide size range of payloads. FIG. 41A shows the encapsulation efficiency of Calcein and FITC-Dextran was 61.9±0.2% and 58.6±1.7%, respectively. FIG. 41B shows that the saRNA encapsulation efficiency of DODAP PF-LNPs, as determined by a RiboGreen™ Assay, was 62.59±0.25%. This is as expected for passively loaded payloads.

FIG. 42 shows HEK-293 cell transfection efficiency of PP75, PLP-NDA or PLP-ADA-coated DODAP PF-LNPs and MC3 LNPs. As shown, the polymer functionalization plays a significant role in enhancing the transfection efficiency of saRNA; as the polymer concentration increased, the transfection efficiency increased by at least one order of magnitude. The PP75 and PLP-ADA coating performed the best compared to PLP-NDA, with a transfection efficiency of ˜10^{7.5} RLU at 1 mg/mL of PP75 or PLP-ADA.

FIG. 43 shows that the relative viability of HEK-293, as determined using Alamar Blue assay, remained above 80% after 48 h of treatment with all the formulations of DODAP PF-LNPs and DODAP LNPs. This confirmed that the PF-LNP formulations had negligible or low cytotoxicity.

### EXAMPLE 12—CO-ENCAPSULATION OF STABILISING MOLECULES IN THE DODAP PF-LNP FORMULATIONS, BOTH IN AQUEOUS SOLUTION AND LYOPHILIZED, FOR LONG-TERM STABLE STORAGE OF RNA AT AMBIENT TEMPERATURES

After optimising the DODAP PF-LNPs with trehalose co-encapsulation, a long-term storage study was carried out to measure the particle size, surface charge, HEK-293 cell viability and HEK-293 cell transfection of the DODAP PF-LNPs co-encapsulated with saRNA and trehalose, over time at different temperatures. Three storage temperatures of −20° C., 4° C. and 20° C. were used to mimic freezer, fridge and ambient conditions. Trehalose is present both inside and outside the PF-LNPs to offer protection from the both sides, ensuring the desirable physical stability (e.g., prevention of particle aggregation and payload leakage) and chemical stability (e.g., protection of biomolecules such as RNA) of the nanoparticle during manufacturing and storage, thus maintaining and prolonging the functionality of saRNA.

FIG. 44 shows the characterization of the formulations, in aqueous solution and lyophilized form, of saRNA-encapsulated DODAP PF-LNPs coated with PP75, PLP-NDA or PLP-ADA at 1 mg/mL, with co-existence of 250 mg/mL trehalose both inside and outside DODAP PF-LNPs (DODAP PF-LNPs/tre). When fresh, the particle size remained below 200 nm and PDI was less than ˜0.4 for all three types of polymer-coated DODAP PF-LNPs. The hydrodynamic size of fresh uncoated DODAP LNPs (DODAP LNPs/tre) at week 0 was above 200 nm, with PDI of 0.3 and 0.4 for aqueous solution and lyophilized form, respectively. After storage at −20° C., 4° C. and 20° C. for 11 weeks DODAP PF-LNPs/tre maintained similar hydrodynamic sizes and PDI compared to the fresh formulations. The uncoated DODAP PF-LNPs/tre showed the most instability with the hydrodynamic size reaching as high as ˜250 nm and PDI 0.6 at all temperatures tested. DODAP PF-LNPs/tre and DODAP LNPs/tre in lyophilized form followed a similar trend to the formulations in aqueous solutions at the different temperatures, except the PDI of DODAP LNPs/tre was less than 0.4 for all temperatures over 8 weeks, suggesting monodispersed particles compared to when in aqueous solution. Overall, DODAP PF-LNPs/tre showed stability in hydrodynamic size and PDI for over 11 weeks, in aqueous solution and lyophilized form.

FIG. 45 shows the zeta potential of DODAP PF-LNPs/tre coated with PP75, PLP-NDA or PLP-ADA at 1 mg/mL, and DODAP LNPs/tre, in aqueous solution and lyophilized form, over 11 weeks. Fresh DODAP PF-LNPs/tre with PP75, PLP-NDA or PLP-ADA and DODAP LNPs/tre in solution had a zeta potential of −15.2±0.3 mV, −266.3±0.8 mV, −23.9±0.5 mV and −7.7±0.9 mV, respectively. The zeta potential of freshly lyophilized DODAP PF-LNPs/tre with PP75, PLP-NDA or PLP-ADA and DODAP LNPs/tre was −16.1±0.2 mV, −24.65±0.4 mV, −29.0±0.2 mV and −8.3±0.8 mV, respectively. The zeta potentials at all temperatures of −20° C., 4° C. and 20° C. remained consistent throughout the 1-week storage for both DODAP PF-LNPs/tre and DODAP LNPs/tre, in aqueous solution and lyophilized form, with very small variance in comparison to the fresh formulations.

FIG. 46 shows the HEK-293 cell transfection efficiency of DODAP PF-LNPs/tre with PP75, PLP-NDA or PLP-ADA at 1 mg/mL, and DODAP LNPs/tre, in aqueous solution and lyophilized form, over 11 weeks of storage. Fresh DODAP PF-LNPs/tre and DODAP LNPs/tre in solution and lyophilized form showed transfection efficiency of ˜10^{7.5} RLU and ˜10⁴ RLU, respectively. The transfection efficiency of the lyophilized DODAP PF-LNPs/tre and DODAP LNPs/tre after 11 weeks of storage at all temperatures of −20° C., 4° C. and 20° C., respectively, remained consistent throughout with small variance in comparison to the fresh formulations, although the DODAP LNPs/tre transfection efficiency generally remained 1-3 orders of magnitude below DODAP PF-LNPs/tre throughout the 1-week period (FIG. 46B). This has validated that, for both DODAP PF-LNPs with polymer functionalization and DODAP LNPs without polymers, coexistence of trehalose both inside and outside nanoparticles can successfully preserve the functionality of saRNA. The excellent thermal stability of RNA and high transfection efficiency even at room (20° C.) temperature particularly for the DODAP PF-LNP formulations, which the inventors have demonstrated, represent another important technological breakthrough in manufacturing, storage and distribution of RNA vaccines and biotherapeutics and have tremendous commercial potential and socioeconomic impact.

Also shown in FIG. 46A, the HEK-293 transfection efficiency of DODAP PF-LNPs/tre and DODAP LNPs/tre in aqueous solution after 11 weeks of storage at −20° C., 4° C. and 200 remained consistent throughout with small variance in comparison to the fresh formulations. The DODAP LNPs/tre transfection efficiency generally remained 1-3 orders of magnitude below DODAP PF-LNPs/tre throughout the 1-week storage in aqueous solution at all those temperatures tested. These are again, significant findings, considering that it is a major challenge to store RNA in aqueous solution. In addition, as with the previous formulations, it is worth pointing out that: (i) the N/P ratio of 1 was used in this preliminary storage work and the use of higher N/P ratios will enhance transfection efficiencies (as shown in FIG. 8 with PF-LNPs); (ii) DODAP PF-LNP compositions and formulations can be further optimized. The inventors believe that thermal stability of RNA formulated in the PF-LNPs after extended storage at ambient temperatures could be further enhanced after optimization.

FIG. 47 shows that the relative viability of HEK-293, as determined using Alamar Blue assay, remained above 80% after 48 h of treatment with all the formulations of DODAP PF-LNPs and DODAP LNPs, both in aqueous solution and lyophilized form, throughout the 1-week storage period at all temperatures of −20° C., 4° C. and 20° C., respectively. These HEK-293 cell viabilities of DODAP PF-LNPs and DODAP LNPs were comparable with the negative control saRNA+tre, the mixture of saRNA and trehalose, further confirming that the PF-LNP formulations had negligible or low cytotoxicity.

Overall, it has been demonstrated that surface functionalization with anionic, viral-peptide-mimicking, endosomolytic polymers, PP75, PLP-NDA and PLP-ADA, plays a vital role in combination with the co-delivery of trehalose to stabilise the RNA-encapsulated PF-LNP formulations composed of cationic and ionizable lipids, both in aqueous solution and lyophilized, for at least 52 weeks at freezer (˜20° C.), fridge (4° C.), room (20° C.) and tropical (40° C.) temperatures, ensuring physical and chemical stability of the RNA delivery nano-formulations and excellent transfection efficiency.

### Conclusions

This invention demonstrates the preparation, characterization and applications of the PF-LNPs for efficient in vitro and in vivo intracellular delivery of nucleic acids including RNA, as well as stable storage at room and tropical temperatures without need for an ultra-cold or cold chain. Physiochemical characterization of the PF-LNPs, PEGylated PF-LNPs, MC3 PF-LNPs and DODAP PF-LNPs has shown average particle sizes of less than 200 nm with zeta potential as low as −41.7±3.7 mV, making this system ideal for the intracellular delivery of RNA vaccines and biotherapeutics. Moreover, saRNA encapsulation efficiencies as high as 87.2±9.8% were achieved, which is ideal to deliver high doses in smaller volumes. Transfection efficiencies 1.5 orders of magnitude higher than the commercially available gold standard, PEI, were achieved. The PF-LNPs can maintain a good balance between protein expression and innate response, showing excellent transfection efficiency in interferon-competent cells. The inventors envision the same to be true for PEGylated PF-LNPs, MC3-PF-LNPs and DODAP PF-LNPs. The anionic, endosomolytic, non-cytotoxic PF-LNPs have shown the improved lymph node drainage, efficient protein expression and excellent immunogenicity in vivo. The inventors envision the same to be true for PEGylated PF-LNPs, MC3-PF-LNPs and DODAP PF-LNPs. The PF-LNP platform is extremely versatile with the demonstrated ability to co-deliver small molecule drugs alongside macromolecules. The unique structure-property relationship of this technology has enabled the inventors to demonstrate that altering the lipid composition to include ionizable lipids (MC3 PF-LNPs and DODAP PF-LNPs) or altering the polymer coating (with PEGylation or PLP-NDA and PLP-ADA) can produce LNPs that are more chemically, physically, and biologically stable than their original PF-LNP counterpart. Existence of stabilising molecules such as trehalose both inside and outside the different PF-LNPs co-encapsulated with RNA can enable stable storage of the formulations, both in aqueous solution and lyophilized, at room and tropical temperatures for at least 52 weeks without need for an ultra-cold or cold chain. Overall, the PF-LNPs are a promising delivery platform suitable for large scale production and can provide a solution to the issue of clinical translation and commercialization of nucleic acids, such as RNA vaccines and biotherapeutics, by offering robust heat-stable formulations for targeted efficient intracellular RNA delivery.

### Materials and Methods

### Materials

1,2-dioleoyl-3-trimethylammonium-propane (DOTAP), 1,2-Dioleoyl-sn-glycero-3-phosphoethanolamine (DOPE), (1,2-dioleoyl-3-dimethylammonium-propane) (DODAP), DLin-MC3-DMA, 1,2-dimyristoyl-rac-glycero-3-methoxypolyethylene glycol-2000 (DMG-PEG2000), 1,2-distearoyl-sn-glycero-3-phosphocholine (DSPC) lipids, cholesterol, iso-phthaloyl chloride, 6-aminofluorescein, Dulbecco's modified Eagle's medium (DMEM), fetal bovine serum (FBS), penicillin and Dulbecco's phosphate-buffered saline (D-PBS) were purchased from Sigma-Aldrich (Dorset, UK). Dimethyl sulfoxide (DMSO), N,N-dimethylformamide (DMF), triethylamine, sodium chloride, 4-dimethylaminopyridine (DMAP), Hoechst 33342, LysoTracker Red DND-99, Alamar Blue Assay Kit, Pierce™ LDH Cytotoxicity Assay Kit and Quant-iT RiboGreen™ Assay Kit were purchased from Fisher Scientific (Loughborough, UK). Anhydrous ethanol, acetone, d₆-DMSO, hydrochloric acid, potassium carbonate, sodium hydroxide, methanol, diethyl ether and chloroform were obtained from VWR (Lutterworth, UK). L-lysine methyl ester dihydrochloride, L-phenylalanine methyl ester hydrochloride and N,N′-dicyclohexylcarbodiimide (DCC) were purchased from Alfa Aesar (Heysham, UK). Defibrinated sheep red blood cells (RBCs) were purchased from TCS Biosciences Ltd (Buckingham, UK). ONE-Glo™ Luciferase Assay Kit was purchased from Promega (Southampton, UK). The polymers, poly(L-lysine isophthalamide) (PLP) and PP75, were synthesized in-house according to the established protocols⁴, saRNA encoding firefly luciferase (fLuc-saRNA) and saRNA encoding hemagglutinin influenza virus (HA-saRNA) were both kindly gifted by Prof. Robin Shattock's group at St Mary Hospital and Department of Infectious Disease, Imperial College London.

### Lipid Nanoparticle (LNP) Synthesis

A lipid film was prepared according to the method used by Guo et al., (2015)¹⁸. Specifically, lipids at specific concentrations were dissolved in chloroform (25% (v/v) methanol). The solvent was removed by rotary evaporation over a 3-hour period. This formed a thin lipid film inside a round bottom flask. The film was hydrated in a water bath at 30° C. for 1 hour.

Extrusion was carried out to synthesize the particles, using a mini extruder (Avanti Polar Lipids Inc., USA). The lipid solution was passed through a 0.2 μm polycarbonate membrane, 31 times, with or without saRNA (or co-encapsulation with other small molecules), to form stable particles. Total lipid concentration in the formulations:
For PF-LNPs: DOTAP: 1.2 mg/mL, DOPE: 1.2 mg/mL, Cholesterol:1.6 mg/mL.
For PEGylated PF-LNPs: DOTAP: 11.1 mg/mL, DOPE: 18.6 mg/mL, Cholesterol: 2.76 mg/mL, DMG-PEG2000: 1.88 mg/mL.
For MC3 PF-LNPs: DLin-MC3-DMA: 16.1 mg/mL, DSPC: 3.67 mg/m, Cholesterol: 7.44 mg/mL, DMG-PEG2000: 1.88 mg/mL.
For DODAP PF-LNPs: DODAP: 11.1 mg/mL, DOPE: 18.6 mg/mL, Cholesterol: 2.76 mg/mL, DMG-PEG2000: 1.88 mg/mL.

### Formation of Polymer-Functionalised Lipid Nanoparticles (PF-LNPs)

To determine polymer coating efficiency, FITC-PP75 was used to coat particles at known concentrations. 10 mg mL⁻¹ stock solution was made using D-PBS, at pH 7.4, which was diluted to desired concentrations. This was mixed with the particle solutions and left to adsorb overnight. The excess PP75 was removed using dialysis devices (Float-A-Lyzer®, MWCO 300 kDa, Spectrumlabs, USA). The fluorescence was measured using a Spectrofluorometer (GloMax®-Multi Detection System, Promega, USA), with excitation wavelength at 490 nm and emission wavelength 510 to 570 nm. A calibration curve was plotted using known concentrations of FITC-PP75 at pH 7.4 to correlate fluorescence readings with concentrations.

### Characterization of PF-LNPs

### Dynamic Light Scattering (DLS)

Dynamic Light Scattering (DLS) (Zetasizer Nano S, Malvern, UK) was used to investigate the change in hydrodynamic size of the PF-LNPs. To prepare the sample for DLS, the PF-LNPs solution was diluted with D-PBS at pH 7.4 and equilibrated for 5 min to obtain an appropriate count rate. The sample was measured at 25° C. with 13 repeats in 10 mm diameter cells, at an angle of 137°.

### Zeta Potential

The zeta potential of the PF-LNPs was measured using a PALS Zeta Potential Analyzer (Brookhaven Instruments Corp., UK). To prepare the sample, the PF-LNPs solution was diluted with D-PBS at pH 7.4 and equilibrated for 5 min to obtain an appropriate count rate. The sample was measured at 20° C. with 6 repeats (20 cycles per run) at a fixed scattering angle of 90° at 659 nm.

### Cells and Culture

HeLa cells (human cervical cells), HEK-293 cells (human embryonic kidney cells) and MRC-5 cells (fibroblasts from lung tissue) were cultured and maintained in complete Dulbecco's Modified Eagle's Medium (cDMEM) (supplemented with 10% (v/v) FBS and 100 UmL⁻¹ penicillin unless specified otherwise). In the case of HEK-293 cells, 5 mg/mL L-glutamine was also added to the culture medium. The cells were trypsinized using trypsin-EDTA and maintained in a humidified incubator with 5% CO₂ at 37° C.

### Quant-iT RiboGreen™ Assay

Encapsulation efficiency of saRNA was measured using RiboGreen™ Assay. saRNA-encapsualted PF-LNPs solutions were diluted to 50 ng mL-1 with a dilution buffer in a 96-well plate. 100 μL RiboGreen dye (Invitrogen, USA) (diluted to 1:1000) was added to each well and the fluorescence intensity was measured using a Spectrofluorometer (GloMax®-Multi Detection System, Promega, USA), following incubation for 5 min, protecting from light. The sample was excited at 480 nm and emission measured at 520 nm. A calibration curve was determined at pH 7.4 to correlate fluorescence readings with concentrations.

### In Vitro Transfection

Cell transfection studies were carried out in HEK-293 and MRC5 cells. 50,000 cells per well were plated in a clear 96-well plate, 48 h before transfection. 100 μL PF-LNPs formulations containing 100 ng of saRNA encoding firefly luciferase (fLuc) were added to each well, containing 50 μL of transfection medium (DMEM with 5 mg/mL L-glutamine). Cells were incubated for 4 h to allow transfection to take place. The medium was then replaced with 100 μL of fresh complete DMEM and left to incubate for 24 h. After 24 h 50 μL of the media was removed and replaced with 50 μL of ONE-Glo™ D-luciferin substrate, and mixed well. 100 μL of the total media was placed in a white 96-well plate and analyzed using a Spectrofluorometer (GloMax®-Multi Detection System, Promega, USA). Background from the media control was subtracted.

Alamar Blue assay

Cell cytotoxicity of the PF-LNPs with and without saRNA was assessed by Alamar Blue assay. Various cell lines were seeded into a 96-well plate (Corning, USA) containing the complete DMEM, 100 μL per well, at a density of 100 cells per well and cultured overnight. The spent medium was replaced with 100 μL PF-LNPs solutions, with and without saRNA, in serum-free DMEM (sterilized with 0.22 μm filter). After incubating for fixed time intervals, the medium was removed and the cells were washed with three times with D-PBS. The cells were then incubated with replenished complete DMEM containing 10% (v/v) Alamar Blue for 4 hours. The fluorescence in each well was measured by the Spectrofluorometer (GloMax®-Multi Detection System, Promega, USA) at the emission wavelength 580-640 nm and excitation wavelength 525 nm. Cytotoxicity was assessed using the fluorescence readings. The concentrations causing 50% inhibition, IC50, were calculated from the concentration-dependent cell viability curves.

### Lactate Dehydrogenase (LDH) Assay

The effect of the PF-LNPs on cell membrane integrity was determined by LDH assay. Various cell lines were seeded into a 96-well plate (Corning, USA) containing complete DMEM (100 μL per well) and cultured for 24 h at a density of 100 cells per well. The spent medium was replaced with 100 μL of the serum-free DMEM containing PF-LNPs solutions, with and without saRNA, in serum-free DMEM (sterilized with 0.22 μm filter). After incubation for 24 h, the released LDH in the supernatant was quantified with the LDH Assay. Briefly, 50 μL of supernatant in each well was transferred into a new plate and mixed with the LDH reaction mixture from the kit. The plate was incubated for 30 min and the reaction was stopped using the stop solution. The absorbance was measured at 490 nm, and at 680 nm (for reference) using the Spectrofluorometer (GloMax®-Multi Detection System, Promega, USA).

### Hemolysis Assay

The endosomolytic behavior of the PF-LNPs was determined by measuring the membrane disruptive behavior using a hemolysis assay. Samples to be tested were prepared at pH 7.4 and pH 6.5 in D-PBS. Defibrinated sheep red blood cells (RBCs) were washed thrice with D-PBS to obtain a pellet of RBCs. The RBCs were mixed into the samples ensuring a concentration of approximately 2×10⁸ RBCs mL⁻¹ was maintained across all samples. This was determined by a calibration curve. The samples were incubated for 1 h at 37° C. in a water bath with gentle agitation. The samples were then centrifuged at 3000 rpm for 3 min and the absorbance of the supernatant from each sampe was measured using a UV-Vis spectrophotometer (GENESYS™ 10S UV-Vis, Thermo Scientific, USA) at 541 nm, and the percentages of relative hemolysis was determined.

### Confocal Microscopy

### Endocytic Mechanism Study

HEK-293 cells were seeded in collagen coated, glass-bottom dishes (MatTek) at 1×10⁵ cells per dish and cultured in an incubator with 5% CO₂ at 37° C. for 24 h. A pathway inhibitor in DMEM was added to each dish for 1 h before adding the inhibitor containing PF-LNPs sample. The cells were incubated for 1 h and then the sample was removed the cells were washed thrice. Cells were then detached using trypsin-EDTA and neutralised using inhibitor containing DMEM. The cells were centrifuged for 5 min at 1000 rpm and supernatant was discarded. Fresh DMEM containing the specific inhibitor was added and cells were imaged using confocal microscopy.

### Animal Studies

In Vivo Protein Expression Female BALB/c mice (Charles River, UK), 6-8 weeks of age, were injected with 50 μL of the PF-LNPs formulations encapsulated with fLuc-saRNA in both hindleg quadriceps. Luciferase expression was then imaged at day 7, as previously determined to be the peak luciferase expression for the VEEV replicon²⁰.

### Immunogenicity

Female BALB/c mice (Charles River, UK), 6-8 weeks of age, were placed into groups of n=5. Group sizes were calculated to detect a difference of 200 ng/mL with a standard deviation of 40 ng/mL, with a power of 0.9 and α=0.05. Mice were injected in one hind leg quadricep muscle with 5 μg of influenza hemagglutinin (HA)-encoding saRNA in 50 μL PF-LNP formulations and boosted with the same formulation after 4 weeks. Tail bleeds were collected before each injection and 2 weeks after the booster injection. Blood was collected and centrifuged at 10,000 rpm for 5 min. The serum was harvested and stored at −20° C. Influenza challenge was introduced at week 7 at which point body weight loss and survival percentage were monitored.

### HA IgG-Specific ELISA

Semiquantitative immunoglobulin IgG ELISA was carried out using the previously described protocol²¹. Briefly, 1 μg/mL recombinant HA in PBS was coated onto ELISA plates. Standards were prepared by coating ELISA plate wells with Lambda (1:1000) light chain and anti-mouse Kappa (1:1000) (Southern Biotech, UK) in PBS. 1% BSA/0.05% Tween-20 in PBS was used to block the plates. After a washing step, diluted samples and purified IgG (Southern Biotech, UK) were added to the plates, starting at 1000 ng/mL and titrating down the plate with five-fold dilution series, followed by 1 h of incubation, and a washing step. A 1:2000 dilution of anti-mouse HA-IgG (Southern Biotech, UK) was used for detection, and plates were developed using TMB (3,3′,5,5′-tetramethylbenidine). After 5 min, the reaction was stopped with a stop solution (Insight Biotechnologies, UK). Absorbance was read on a spectrophotometer (VersaMax, Molecular Devices) with SoftMax Pro GxP v5 software.

### Lymph Node Histology Study

Female BALB/c mice (Charles River, UK), 6-8 weeks of age, were placed into groups of n=3. Mice were injected in one hind leg quadricep muscle with 5 μg of EGFP-saRNA in 50 μL PF-LNP formulations coated with Cy5-PP75. Drainage of the formulations into the lymph nodes was allowed for 1 h before harvesting and fixing in 15% then 30% sucrose solution. The lymph nodes were then frozen embedded in Optimal Cutting Temperature (OCT) at −20° C. and sliced 5 μm using a rotary microtome (CryoStar NX70 Cryostat, U.K.). The samples were collected on POLYSINE™ microscope glass slides (Thermo Fisher Scientific, U.K.) and left to air dry for 1 h. Next, the sample slides were rinsed in tap water, left to stained in Oil Red O filtered solution for 20 min and washed in distilled water. Then a counterstain in gill 1 haematoxylin for 1 to 2 min followed by washing in tap water. Next the sample slides were differentiated for a few seconds in 1% acid alcohol, washed and blue in running tap water. Finally, the samples were rinsed in distilled water and a cover slip in an aqueous apathy's medium was applied as a mountant on top of the slides. The samples were left to dry overnight and an Inverted Wide field Microscope (Zeiss Axio Observer, U.K.) was used to image the lymph node samples.

### Lyophilization

The saRNA-encapsulated PF-LNP formulations with co-existence of trehalose both inside and outside the PF-LNPs were synthesised according to the protocols outlined above. The formulations were then frozen to −80° C. and lyophilized overnight. The dehydrated samples were rehydrated using PBS and mixed using a vortex mixer (Velp F202A0175 Wizard Vortex Mixer, U.K.).

### Statistical Analysis

All data points were repeated in triplicate (n=3). Results are presented as mean values with standard deviation encompassing at least 95% confidence interval. The Student's t-test was performed to evaluate the statistical significance; P<0.05 was considered to be statistically significant.

### REFERENCES

1. Fleeton, M. N. et al. Self-Replicative RNA Vaccines Elicit Protection against Influenza A Virus, Respiratory Syncytial Virus, and a Tickborne Encephalitis Virus. J. Infect. Dis. 183, 1395-1398 (2001).
2. Lundstrom, K. Self-replicating RNA viruses for RNA therapeutics. Molecules (2018) doi:10.3390/molecules23123310.
3. Chen, R., Khormaee, S., Eccleston, M. E. & Slater, N. K. H. The role of hydrophobic amino acid grafts in the enhancement of membrane-disruptive activity of pH-responsive pseudo-peptides. Biomaterials 30, 1954-1961 (2009).
4. Chen, R., Eccleston, M. E., Yue, Z. & Slater, N. K. H. Synthesis and pH-responsive properties of pseudo-peptides containing hydrophobic amino acid grafts. J. Mater. Chem. (2009) doi:10.1039/b902822f.
5. Eccleston, M. E., Slater, N. K. H. & Tighe, B. J. Synthetic routes to responsive polymers; co-polycondensation of tri-functional amino acids with diacylchlorides. React. Funct. Polym. 42, 147-161 (1999).
6. Eccleston, M. E., Kuiper, M., Gilchrist, F. M. & Slater, N. K. H. pH-responsive pseudo-peptides for cell membrane disruption. J. Control. Release 69, 297-307 (2000).
7. Chen, R., Yue, Z., Eccleston, M. E., Williams, S. & Slater, N. K. H. Modulation of cell membrane disruption by pH-responsive pseudo-peptides through grafting with hydrophilic side chains. J. Control. Release (2005) doi:10.1016/j.jconrel.2005.07.011.
8. Ho, V. H. B., Slater, N. K. H. & Chen, R. PH-responsive endosomolytic pseudo-peptides for drug delivery to multicellular spheroids tumour models. Biomaterials 32, 2953-2958 (2011).
9. Chen, S. & Chen, R. A Virus-Mimicking, Endosomolytic Liposomal System for Efficient, pH-Triggered Intracellular Drug Delivery. ACS Appl. Mater. Interfaces 8, 22457-22467 (2016).
10. Rejman, J., Oberle, V., Zuhorn, I. S. & Hoekstra, D. Size-dependent internalization of particles via the pathways of clathrin-and caveolae-mediated endocytosis. Biochem. J. (2004) doi:10.1042/BJ20031253.
11. Nakamura, T. et al. The Effect of Size and Charge of Lipid Nanoparticles Prepared by Microfluidic Mixing on Their Lymph Node Transitivity and Distribution. Mol. Pharm. 17, 944-953 (2020).
12. Tribet, C. & Vial, F. Flexible macromolecules attached to lipid bilayers: Impact on fluidity, curvature, permeability and stability of the membranes. Soft Matter (2007) doi:10.1039/b708431p.
13. Gutierrez De Rubalcava, C. et al. Interactions between liposomes and hydroxypropylmethylcellulose. Int. J. Pharm. (2000) doi:10.1016/S0378-5173(00)00433-6.
14. Liu, D. Z., Chen, W. Y., Tasi, L. M. & Yang, S. P. Microcalorimetric and shear studies on the effects of cholesterol on the physical stability of lipid vesicles. Colloids Surfaces A Physicochem. Eng. Asp. 172, 57-67 (2000).
15. Wu, G. & Lee, K. Y. C. Effects of poloxamer 188 on phospholipid monolayer morphology: An atomic force microscopy study. Langmuir (2009) doi:10.1021/la802908x.
16. Guan, S. & Rosenecker, J. Nanotechnologies in delivery of mRNA therapeutics using nonviral vector-based delivery systems. Gene Ther. 24, 133-143 (2017).
17. Geall, A. J. et al. Nonviral delivery of self-amplifying RNA vaccines. Proc. Natl. Acad. Sci. 109, 14604-14609 (2012).
18. Guo, F., Yu, M., Wang, J., Tan, F. & Li, N. Smart IR780 Theranostic Nanocarrier for Tumor-Specific Therapy: Hyperthermia-Mediated Bubble-Generating and Folate-Targeted Liposomes. ACS Appl. Mater. Interfaces 7, 20556-20567 (2015).
19. Blakney, A. K., McKay, P. F. & Shattock, R. J. Structural Components for Amplification of Positive and Negative Strand VEEV Splitzicons. Front. Mol. Biosci. (2018) doi:10.3389/fmolb.2018.00071.
20. Vogel, A. B. et al. Self-Amplifying RNA Vaccines Give Equivalent Protection against Influenza to mRNA Vaccines but at Much Lower Doses. Mol. Ther. 26, 446-455 (2018).
21. Badamchi-Zadeh, A. et al. Intramuscular immunisation with chlamydial proteins induces Chlamydia trachomatis specific ocular antibodies. PLoS One (2015) doi:10.1371/journal.pone.011209.

1. A sub-micron particle comprising a first payload molecule, a lipid structure and a plurality of amphiphilic polymer chains surrounding the lipid structure, wherein the first payload molecule is a macromolecule, optionally a nucleic acid, and the hydrophilicity of the amphiphilic polymer chains changes in response to an external stimulus.

2. The sub-micron particle according to claim 1, wherein the first payload molecule is encapsulated in the lipid structure, and alternatively or additionally, covalently conjugated and/or physically attached to the outer surface of the lipid structure; and/or
wherein the first payload molecule is a nucleic acid, a peptide, an affimer, a protein, an antibody or a fragment thereof, a glycoprotein, a lipopolysaccharide, a carbohydrate, a lipid or a macrocycle, and is optionally a nucleic acid, and the nucleic acid is DNA, RNA or a DNA/RNA hybrid sequence, optionally wherein the RNA is self-amplifying RNA (saRNA) or messenger RNA (mRNA);
and/or
wherein the lipid structure is a lipid nanoparticle or a liposome, and is optionally a lipid nanoparticle.

3-6. (canceled)

7. The sub-micron particle according to claim 1, wherein the lipid structure comprises a plurality of lipids; optionally wherein at least one of the plurality of lipids comprises a cationic or ionizable lipid; optionally wherein the cationic or ionizable lipid:
(i) is a multivalent cationic lipid, or is a pH-sensitive lipid;
(ii) comprises a positively charged or ionizable nitrogen atom; and/or
(iii) displays a positive charge in an acidic solution.

8-9. (canceled)

10. The sub-micron particle according to claim 7, wherein at least one of the plurality of lipids comprises a cationic or ionizable lipid and the cationic or ionizable lipid is dioleoyl-3-trimethylammonium propane (DOTAP), 1,2-di-O-octadecenyl-3-trimethylammonium propane (DOTMA), an ethylphosphatidylcholine (ethyl PC), didodecyldimethylammonium bromide (DDAB), 3β-[N—(N′,N′-dimethylaminoethane)-carbamoyl]cholesterol (DC-Cholesterol), N4-Cholesteryl-Spermine (GL67), 1,2-dioleyloxy-3-dimethylaminopropane (DODMA), DLin-MC3-DMA, 1,2-dioleoyl-3-dimethylammonium-propane (DODAP), or heptadecan-9-yl 8-((2-hydroxyethyl) (6-oxo-6-(undecyloxy) hexyl) amino) octanoate (SM-102); and/or
wherein the plurality of lipids comprises one or more helper lipids, optionally wherein:
(i) the helper lipids are zwitterionic, optionally a phosphatidylcholine selected from DOPC, DSPC, DPPC, DDPC, DLPC, DMPC, POPC, DEPC, and L-α-phosphatidylcholine; or a phosphatidylethanolamine selected from DOPE, DMPE, DPPE, and DSPE; or
(ii) the helper lipids are non-zwitterionic, optionally a phosphatidylglycerol selected from DOPG, DMPG, DPPG, DSPG, and POPG), a phosphatidylserine (including DOPS) or a phosphatidic acid selected from DMPA, DPPA, and DSPA.

11. (canceled)

12. The sub-micron particle according to claim 7, wherein the sub-micron particle does not contain or comprise zwitterionic lipids.

13. The sub-micron particle according to claim 7, wherein at least one of the plurality of lipids comprises a zwitterionic lipid, optionally wherein the zwitterionic lipid comprises a positively charged nitrogen atom and/or a negatively charged oxygen atom.

14. (canceled)

15. The sub-micron particle according to claim 7, wherein at least one of the plurality of lipids comprises a cationic or ionizable lipid and one or more helper lipids and wherein the weight ratio of the cationic or ionizable lipid to the helper lipid is between 20:1 and 1:20, between 10:1 and 1:10, between 5:1 and 1:5, between 4:1 and 1:4, between 3:1 and 1:3, between 2:1 and 1:2, between 1.5:1 and 1:1.5 or between 1.8:1 and 1:1.8, optionally about 1:1; and/or
wherein the plurality of lipids comprises at least 5 wt %, at least 10 wt %, at least 20 wt %, at least 30 wt % or at least 40 wt % sterol, optionally wherein the sterol is or comprises a C₁₋₂₄ alkyl phytosterol, stigmasterol or stigmastanol, and is optionally cholesterol; and/or
wherein at least one of the plurality of lipids comprise a PEGylated lipid, optionally wherein the PEGylated lipid comprises between 0.1 and 50 wt %, between 0.5 and 40 wt %, between 1 and 30 wt %, between 2 and 20 wt %, between 3 and 10 wt %, between 4 and 8 wt % or between 5 and 7 wt % of the plurality of lipids.

16-18. (canceled)

19. The sub-micron particle according to claim 1, wherein the sub-micron particle has an N/P ratio of between 1:100 and 100:1, between 1:50 and 80:1, between 1:10 and 60:1, between 1:5 and 50:1, between 1:3 and 40:1, between 1:2 and 30:1, between 1:1 and 25:1, between 2:1 and 20:1, between 5:1 and 15:1, or between 8:1 and 12:1; and/or
wherein the external stimulus is:
(i) a chemical stimulus, which is selected from a change in pH, a specific redox potential, a specific ion or a specific gas;
(ii) a physical stimulus, which is selected from a temperature variation, a change in light or an electromagnetic field; or
(iii) a biochemical stimulus, which is selected from a protein, a peptide, an enzyme, a glucose or a nucleic acid, such as DNA; and/or
wherein the amphiphilic polymer chains are negatively charged at a pH of at least 4 at 20° C., at least 5 at 20° C., at least 6 at 20° C., at least 6.5 at 20° C. or at least 7 at 20° C.; and/or
wherein the amphiphilic polymer chains have a number average molecular weight of at least 1 kDa, at least 2 kDa, at least 5 kDa, at least 10 kDa, at least 15 kDa, at least kDa, at least 22 kDa, at least 24 kDa, at least 24.5 kDa or at least 24.8 kDa, or less than 250 kDa, less than 100 kDa, less than 75 kDa, less than 50 kDa, less than 40 kDa, less than 30 kDa, less than 28 kDa, less than 26 kDa, less than 25.5 kDa or less than 25 kDa; and/or
wherein the sub-micron particle comprises at least one targeting ligand or moiety, is or comprises at least one of a peptide, a protein, an aptamer, a carbohydrate, an oligosaccharide, a folic acid or folate, and antibody or an antigen binding fragment thereof, a vitamin or a derivative thereof.

20-21. (canceled)

22. The sub-micron particle according to claim 1, wherein the amphiphilic polymer chains comprise one or more mers of formula I: 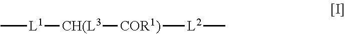
wherein L¹ comprises one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴; L² and L³ are independently absent or comprise one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴;
each R¹ is independently NR⁶R⁷, OR¹⁰ or OH;
R⁴ is H, an optionally substituted C₁₋₃₀ alkyl, an optionally substituted C₂₋₃₀ alkenyl or an optionally substituted C₂₋₃₀ alkynyl;
R⁶ and R⁷ are each independently H, an optionally substituted C₁₋₃₀ alkyl, an optionally substituted C₂₋₃₀ alkenyl or an optionally substituted C₂₋₃₀ alkynyl; and
R¹⁰ is an optionally substituted C₁₋₃₀ alkyl, an optionally substituted C₂₋₃₀ alkenyl or an optionally substituted C₂₋₃₀ alkynyl; optionally
wherein at least one of the mers of formula I, R¹ is OH; and/or
wherein the amphiphilic polymer chains comprise one or more mers of formula II: 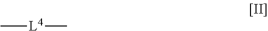
wherein L⁴ comprises one or more linker elements selected from the group consisting of an optionally substituted C₁₋₃₀ alkylene, an optionally substituted C₂₋₃₀ alkenylene, an optionally substituted C₂₋₃₀ alkynylene, an optionally substituted C₃₋₂₀ cycloalkylene, an optionally substituted C₃₋₂₀ cycloalkenylene, an optionally substituted C₃₋₂₀ cycloalkynylene, an optionally substituted C₃₋₁₂ heterocyclylene, an optionally substituted C₆₋₂₀ arylene, an optionally substituted C₅₋₁₀ heteroarylene, CO, O, S and NR⁴.

23-24. (canceled)

25. The sub-micron particle according to claim 22, wherein the amphiphilic polymer chains are or comprise a plurality of polymers of formula III: 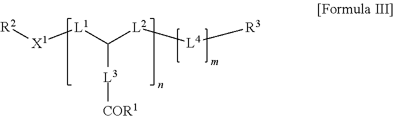
wherein
X¹ is absent or is O, S or NR⁵;
R² is H, an optionally substituted C₁₋₃₀ alkyl, an optionally substituted C₂₋₃₀ alkenyl, an optionally substituted C₂₋₃₀ alkynyl, an optionally substituted C₆₋₂₀ aryl, an optionally substituted C₃₋₂₀ cycloalkyl, an optionally substituted C₃₋₂₀ cycloalkenyl, an optionally substituted C₃₋₂₀ cycloalkynyl, an optionally substituted 5 to 20 membered heteroaryl or an optionally substituted 3 to 20 membered heterocycle;
R³ and R⁵ are each independently H, an optionally substituted C₁₋₃₀ alkyl, an optionally substituted C₂₋₃₀ alkenyl or an optionally substituted C₂₋₃₀ alkynyl; and
n is an integer and is at least 1; and
m is 0 or is an integer which is at least 1; optionally
wherein:
(i) there is one or more -L⁴- blocks, which may be same or different from each other;
(ii) there is one or more -L¹-CH(L³-COR¹)-L²- blocks, which may be same or different from each other; and/or
(iii) the polymers have a random or controlled sequence; or
wherein the amphiphilic polymer chains are or comprise a plurality of polymers of formula IV: 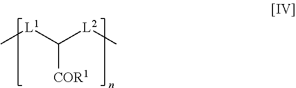
optionally wherein the polymer of formula IV has formula IVa:

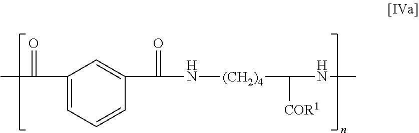

26-28. (canceled)

29. The sub-micron particle according to claim 22, wherein between 1 and 99%, between 5 and 85%, between 10 and 60% or between 15 and 40% of the R¹ group in the polymers are OH, and between 1 and 99%, between 15 and 95% of the R¹ group in the polymers are NR⁶R⁷ or OR¹⁰; optionally wherein between 1 and 99%, between 15 and 95%, between 40 and 90% or between 60 and 85% of the R¹ group in the polymers are NR⁶R⁷, R⁶ is H and R⁷ is 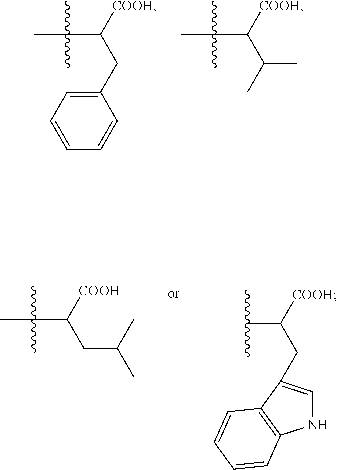
or
between 1 and 99%, between 5 and 90%, between 10 and 80%, between 12.5 and 70% or between 15 and 65% of the R¹ group in the polymers are NR⁶R⁷, R⁶ is H and R⁷ is —(CH₂)₆CH₃,
(CH₂)₉CH₃, —(CH₂)₁₃CH₃, —(CH₂)₁₇CH₃ or —(CH₂)₁₁COOH.

30-32. (canceled)

33. The sub-micron particle according to claim 25, wherein:
(i) n is an integer of at least 1, at least 2, at least 3, at least 4, at least 5, at least 6, at least 7 or at least 8;
(ii) n is an integer of less than 50, less than 40, less than 30, less than 25, less than 20, less than 15, less than 12 or less than 10;
(iii) n is an integer between 1 and 50, between 2 and 40, between 3 and 30, between 4 and 25, between 5 and 20, between 6 and 15, between 7 and 12 or between 8 and 10;
(iv) n is an integer of at least 5, at least 10, at least 15, at least 25, at least 50, at least 75, at least 90 or at least 95. n may be an integer of less than 5,000, less than 1,000, less than 500, less than 250, less than 150, less than 125, less than 110 or less than 105. n may be an integer between 5 and 5,000, between 10 and 1,000, between 15 and 500, between 25 and 250, between 50 and 150, between 75 and 125, between 90 and 110 or between 95 and 105.
(v) n is an integer of between 10 and 1,000, between 15 and 750, between 25 and 500, between 40 and 250, between 60 and 200, between 90 and 170, between 110 and 150, or between 120 and 140;
(vi) n is an integer of at least 100, at least 250, at least 500, at least 750, at least 1,000, at least 1,250 or at least 1,500;
(vii) m is an integer of at least 5, at least 10, at least 15, at least 20, at least 25, at least 30, at least 35, at least 40 or at least 45;
(viii) m is an integer of less than 1,000, less than 500, less than 250, less than 150, less than 100, less than 80, less than 70, less than 60 or less than 55; or
(ix) m is an integer between 5 and 1,000, between 10 and 500, between 15 and 250, between 20 and 150, between 25 and 100, between 30 and 80, between 35 and 70, between 40 and 60 or between 45 and 55.

34. The sub-micron particle according to claim 1, wherein the polymer is polymer L₁₀₀F₅₀, having the structure:

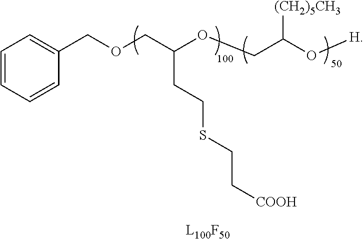

35. The sub-micron particle according to claim 1, wherein the sub-micron particle comprises a second payload molecule, optionally
wherein the second payload molecule is encapsulated in the lipid structure, and alternatively or additionally, covalently conjugated and/or physically attached to the outer surface of the lipid structure; and/or
wherein the second payload molecule is an active pharmaceutical ingredient (API), or a component thereof, or facilitator or enabler thereof, or a macromolecule or a small molecule, optionally wherein the second payload molecule has a molecular weight of less than 900 daltons, less than 800 daltons, less than 700 daltons, less than 600 daltons, less than 500 daltons or less than 400 daltons.

36. (canceled)

37. The sub-micron particle according to claim 1, wherein the sub-micron particle comprises at least one stabilizing molecule; optionally wherein the or each stabilizing molecules may be a carbohydrate and/or a polyol; optionally wherein
(i) the carbohydrate is a monosaccharide, which is selected from a group consisting of: glucose; galactose; fructose; mannose; and xylose or a pharmaceutically acceptable complex, salt, solvate, tautomeric form, stereoisomer or polymorphic form thereof;
(ii) the carbohydrate may be a disaccharide, which is selected from a group consisting of: trehalose; sucrose; lactose; maltose; isomaltose; lactitol; lactulose; mannobiose; and isomalt or a pharmaceutically acceptable complex, salt, solvate, tautomeric form, stereoisomer or polymorphic form thereof;
(iii) the carbohydrate is a trisaccharide, which is selected from a group consisting of: nigerotriose; maltotriose: melezitose; maltotriulose; raffinose; and kestose or a pharmaceutically acceptable complex, salt, solvate, tautomeric form, stereoisomer or polymorphic form thereof; or
(iv) the carbohydrate is a polysaccharide, which is selected from the group consisting of: dextran; amylose: amylopectin; glycogen; galactogen; inulin; callose; cellulose: chitosan; and chitin or a pharmaceutically acceptable complex, salt, solvate, tautomeric form, stereoisomer or polymorphic form thereof; or
wherein
the carbohydrate is trehalose, or a pharmaceutically acceptable complex, salt, solvate, tautomeric form, stereoisomer or polymorphic form thereof.

38-41. (canceled)

42. A method of producing a sub-micron particle, the method comprising combining a first payload molecule, a lipid structure and a plurality of amphiphilic polymer chains to produce the sub-micron particle, wherein the first payload molecule is a macromolecule, optionally a nucleic acid, and the hydrophilicity of the amphiphilic polymer chains changes in response to an external stimulus.

43. The method according to claim 42, wherein the method comprises contacting the first payload molecule and a plurality of lipids to produce a lipid structure encapsulating, or being covalently conjugated and/or physically attached onto its outer surface with, the first payload molecule, and subsequently contacting the lipid structure and the plurality of amphiphilic polymer chains to produce the sub-micron particle; and/or
wherein the method comprises contacting the first payload molecule, a second payload molecule and a plurality of lipids to produce a lipid structure encapsulating, or being covalently conjugated and/or physically attached onto its outer surface with, the first and second payload molecules; and subsequently contacting the lipid structure and the plurality of amphiphilic polymer chains to produce the sub-micron particle; and/or
wherein the method is used to modify the existing lipid structure, including lipid nanoparticle and liposome systems.

44-46. (canceled)

47. A composition comprising a plurality of sub-micron particles of claim 1, optionally wherein the composition is a pharmaceutical composition and comprises a pharmaceutically acceptable vehicle.

48-49. (canceled)

50. A vaccine composition comprising the sub-micron particle of claim 1.

51. (canceled)

52. A method of vaccinating a subject, the method comprising administering, or having administered, to a subject in need thereof, a therapeutic amount of the sub-micron particle of claim 1.
